SEQUENCE LISTING (101 sequences)

SEQ ID NO: 1 - DNA, 1908 bp, Aspergillus fumigatus
gttcgacgtcattgccacgtttcgacccaagggcagacgccatgtcgccgagcgatcgccgcgatatgcctcgaatttgcgccattcggcatccagtttccagtgcccttccccgaatgactgtctccactattcggcaagattgtaaatcaagcctgaagaagcggagcaattcttggaagtcgtatgttctactgatttctgtgcctggcgcagacgggtatataaataaagatcaccgcaccgaggagtttcttaccaacccatcaataaccatccacaatctcctacaacaaaaatgactgtcgccgatatcgacgttcctcctgccgagggcatcccctacttcactccggcccagaaccctcctgccggtacggcagctaacccccagaccaatggccagaagatccccaagctcttcacgcccttgaccatccgtggcgtcaccttccagaaccgccttggtgtaagtccgtttgcccttgctcatatcgacgaaagctaatcccccgtcagctcgcgcccctctgccaatactccgcccaggacggccacatgaccgactaccacatcgcccatctgggtgggatcgcccaacgcggacccggcctgatgctgattgaggcgaccgccgtccagcccgaaggccgcatcacccctcaggatgtcggtctgtggaaggactcccagatcgccccgatgcgccgggtcatcgacttcgtgcacagccagggccagaagatcggcgtgcagcttgcccatgccggccggaaagccaccaccgttgcgccctggatctcattctcggccatcgcgacggagaaggtcggcggatggccggaccgcgtcaaagggcccggcgatatcccctttgcggagcccttcgccaagcccaaggccatgacgctggatgagatcgagcagttcaagaaggactgggtggcggccacgaagcgcgccatcgccgccggtgcggactttgtcgagattcacaatgcgcatggatacctgctgtcgtcattcctctcgccggccgccaacaaccgcacggaccagtacggcgggtcgttcgagaaccgcatccggctgtctctcgagattgcgcagttgactcgggacgccgtcggccctcatgtgcccgttttcctgcgcatttcggcctcggactggtgcgaggagaccctgccggagcagagctggaagtcggaggataccgtgcggttcgcgcaggagctggtcaagcagggcgccgttgatctgatcgatatcagcagcggtggtgttctcgcgcagcagaagatcaagtccggccctgccttccaggtgccttttgccgtggccgtgaagaaggccgtcggcgacaagctgctggttgccgccgtgggtgccatcaccaacggcaagcaggcgaatcagattctagaggagcaggatatcgacgttgcgctggttggccgtgggttccagaaggatcccggtctggcctggacgtttgctcagcacctcggcgtcgaaatctccatggccaaccagatccgctggggcttcacccggcgtggaggcaccccgtacattgatccttcggtgtacaagcagtctattttcgatgtatagagtatagatagagttgaagatgatacctcatagacgatcaatggacccttgcatattatttctcgtctcctgcgtatgttcaaggtattcacagtagctgcgtcctcttaagtttctccgtcattcgttctattctactccaatcgcaacgcatggcgaccacggatcgagtcgaatttctccgtcgttcgtatctgatcaatataaaaagcggggaatggcttgaccccgcgcagaatgtcgatctcttcgcaaactctcggtgtataggacgctcagcaacgatcaagg

SEQ ID NO: 2 - DNA, 1445 bp, Aspergillus fumigatus
gtatgttctactgatttctgtgcctggcgcagacgggtatataaataaagatcaccgcaccgaggagtttcttaccaacccatcaataaccatccacaatctcctacaacaaaaatgactgtcgccgatatcgacgttcctcctgccgagggcatcccctacttcactccggcccagaaccctcctgccggtacggcagctaacccccagaccaatggccagaagatccccaagctcttcacgcccttgaccatccgtggcgtcaccttccagaaccgccttggtctcgcgcccctctgccaatactccgcccaggacggccacatgaccgactaccacatcgcccatctgggtgggatcgcccaacgcggacccggcctgatgctgattgaggcgaccgccgtccagcccgaaggccgcatcacccctcaggatgtcggtctgtggaaggactcccagatcgccccgatgcgccgggtcatcgacttcgtgcacagccagggccagaagatcggcgtgcagcttgcccatgccggccggaaagccaccaccgttgcgccctggatctcattctcggccatcgcgacggagaaggtcggcggatggccggacccgcgtcaaagggcccggcgatatcccctttgcggagcccttcgccaagcccaaggccatgacgctggatgagatcgagcagttcaagaaggactgggtggcggccacgaagcgcgccatcgccgccggtgcggactttgtcgagattcacaatgcgcatggatacctgctgtcgtcattcctctcgccggccgccaacaaccgcacggaccagtacggcgggtcgttcgagaaccgcatccggctgtctctcgagattgcgcagttgactcgggacgccgtcggccctcatgtgcccgttttcctgcgcatttcggcctcggactggtgcgaggagaccctgccggagcagagctggaagtcggaggataccgtgcggttcgcgcaggagctggtcaagcagggcgccgttgatctgatcgatatcagcagcggtggtgttctcgcgcagcagaagatcaagtccggccctgccttccaggtgccttttgccgtggccgtgaagaaggccgtcggcgacaagctgctggttgccgccgtgggtgccatcaccaacggcaagcaggcgaatcagattctagaggagcaggatatcgacgttgcgctggttggccgtgggttccagaaggatcccggtctggcctggacgtttgctcagcacctcggcgtcgaaatctccatggccaaccagatccgctggggcttcacccggcgtggaggcaccccgtacattgatccttcggtgtacaagcagtctattttcgatgtatagagtatagatagagttgaagatgatacctcatagacgatcaatggacccttgcatattattt

SEQ ID NO: 3 - protein, 422 aa, Aspergillus fumigatus
MTVADIDVPPAEGIPYFTPAQNPPAGTAANPQTNGQKIPKLFTPLTIRGVTFQNRLGLAPLCQYSAQDGHMTDYHIAHLGGIAQRGPGLMLIEATAVQPEGRITPQDVGLWKDSQIAPMRRVIDFVHSQGQKIGVQLAHAGRKATTVAPWISFSAIATEKVGGWPDRVKGPGDIPFAEPFAKPKAMTLDEIEQFKKDWVAATKRAIAAGADFVEIHNAHGYLLSSFLSPAANNRTDQYGGSFENRIRLSLEIAQLTRDAVGPHVPVFLRISASDWCEETLPEQSWKSEDTVRFAQELVKQGAVDLIDISSGGVLAQQKIKSGPAFQVPFAVAVKKAVGDKLLVAAVGAITNGKQANQILEEQDIDVALVGRGFQKDPGLAWTFAQHLGVEISMANQIRWGFTRRGGTPYIDPSVYKQSIFDV

SEQ ID NO: 4 - DNA, 1352 bp, Aspergillus fumigatus
atgtcgcaacctgttgtgcctgacatcgagaacaaacccgcgccgggtatctcgtactttactccggcgcaagagccgcctgctggcaccgctgctaatcctcagtctgatggatcggcacctcccaagctcttccggccgctttcggtgcggggtctgacctttcacaatcgcattggcgtgagtgcagtccaggcaattatgctatccatcctatgcgagcccttgcattggaacagccgcttacagggaatgataatgagtagctatcgccactctgccaatactcagccgacgatggacacatgactccctggcatatggcacatcttggagggattgcccagcgagggccaggattcttgatggtcgaggcaacagcagtcgaaccggaaggcaggatcaccccgcaggacctgggactatggaaagactcgcagattgagccattgagccgcgtgatcgagtttgtccacagtcagaaccagcttatcggcgtgcagatcgcacacgcaggtcgcaaggccagcaccgtcgcgccatggctctcggccaacgataccgcctccgagaagatgggcggctggccaggccgcgtcaaaggcccgacaaatgtgcccttcaccgttaagaaccctgtgccgaaggagatgaccaagcaggatatcgaggatctgaagaccgcctgggtggccgctgtcaaacgggctgttaaggccggagccgactttatcgagatccacaatgcgcatggctatcttctgatgtcgttcctctcccctgcggtcaacacgagaacagacgagtacggaggcagttttgagaatcgcatccggctcagtctggagatcgccaagctcacccgcgaaaatgtgcccaaggatatgcctgtcttcctgcgggtctccgccaccgattggctggaggaggtgcagccgaacaagcccagctggcgaggcgtggacactgtccgatttgcgaagatcctggcagaaacgggttacgttgacgtgcttgacgtgagcagtggcggcactcattcggagcagcatatccacgcgaagccaggcttccaggcaccctttgctattgccgtcaagaacgccgtcggggacaaactcgcagtggcatcagtgggtatgattgccagcgcgcatttggccaattccttgttggagaaggacggactggaccttgtgctggttggacgtggcttccagaagaacccggggctggtgtgggcgtgggccgacgagctgaatgtagagatctccatggctaatcagatccgatggggtttctcgcggcgcggtgctggtccttacctcaggaagaaactcgagaagatataa

SEQ ID NO: 5 - DNA, 1266 bp, Aspergillus fumigatus
atgtcgcaacctgttgtgcctgacatcgagaacaaacccgcgccgggtatctcgtactttactccggcgcaagagccgcctgctggcaccgctgctaatcctcagtctgatggatcggcacctcccaagctcttccggccgctttcggtgcggggtctgacctttcacaatcgcattggcctatcgccactctgccaatactcagccgacgatggacacatgactccctggcatatggcacatcttggagggattgcccagcgagggccaggattcttgatggtcgaggcaacagcagtcgaaccggaaggcaggatcaccccgcaggacctgggactatggaaagactcgcagattgagccattgagccgcgtgatcgagtttgtccacagtcagaaccagcttatcggcgtgcagatcgcacacgcaggtcgcaaggccagcaccgtcgcgccatggctctcggccaacgataccgcctccgagaagatgggcggctggccaggccgcgtcaaaggcccgacaaatgtgcccttcaccgttaagaaccctgtgccgaaggagatgaccaagcaggatatcgaggatctgaagaccgcctgggtggccgctgtcaaacgggctgttaaggccggagccgactttatcgagatccacaatgcgcatggctatcttctgatgtcgttcctctcccctgcggtcaacacgagaacagacgagtacggaggcagttttgagaatcgcatccggctcagtctggagatcgccaagctcacccgcgaaaatgtgcccaaggatatgcctgtcttcctgcgggtctccgccaccgattggctggaggaggtgcagccgaacaagcccagctggcgaggcgtggacactgtccgatttgcgaagatcctggcagaaacgggttacgttgacgtgcttgacgtgagcagtggcggcactcattcggagcagcatatccacgcgaagccaggcttccaggcaccctttgctattgccgtcaagaacgccgtcggggacaaactcgcagtggcatcagtgggtatgattgccagcgcgcatttggccaattccttgttggagaaggacggactggaccttgtgctggttggacgtggcttccagaagaacccggggctggtgtgggcgtgggccgacgagctgaatgtagagatctccatggctaatcagatccgatggggtttctcgcggcgcggtgctggtccttacctcaggaagaaactcgagaagatataa

SEQ ID NO: 6 - protein, 421 aa, Aspergillus fumigatus
MSQPVVPDIENKPAPGISYFTPAQEPPAGTAANPQSDGSAPPKLFRPLSVRGLTFHNRIGLSPLCQYSADDGHMTPWHMAHLGGIAQRGPGFLMVEATAVEPEGRITPQDLGLWKDSQIEPLSRVIEFVHSQNQLIGVQIAHAGRKASTVAPWLSANDTASEKMGGWPGRVKGPTNVPFTVKNPVPKEMTKQDIEDLKTAWVAAVKRAVKAGADFIEIHNAHGYLLMSFLSPAVNTRTDEYGGSFENRIRLSLEIAKLTRENVPKDMPVFLRVSATDWLEEVQPNKPSWRGVDTVRFAKILAETGYVDVLDVSSGGTHSEQHIHAKPGFQAPFAIAVKNAVGDKLAVASVGMIASAHLANSLLEKDGLDLVLVGRGFQKNPGLVWAWADELNVEISMANQIRWGFSRRGAGPYLRKKLEKI

SEQ ID NO: 7 - DNA, 1329 bp, Aspergillus fumigatus
atgggttccaacgccttccggtcccccgccgtcaccaagtcctcctccaccccctactacactcccgccaacaatggaggcgccgccctgcaccccgacgaccccacgacccctacgctcttccggcccttacaaatccgcaatgtgacgctcaagaaccgcatcatggtgtcgcccatgtgcatgtactcctgcgagtcggacccgtcgtctccccacgtcggcgccctaacaaactaccacctggcgcatctgggccacctcgccctcaaaggcgcaggcctcgtcttcatcgaagccaccgccgtgcagcccaacgggcgcatctcccccaacgactcgggcctctggcaggacggcaccacctcggaacaattcctggggctgaagcgggtcgtcgagttcatgcacgcacagggcgccaaggtcgggatccagcttgcgcatgcgggccggaaagcgagtgccgttgcgccgtggctggcggcgcaggcgggcaagtcgagtctgaaggcggatgagagcgttggcgggtggcccgcggatgtggtgggtccgtcgggcggggaggagcatatctttagtcccgaggaggatgcgtattgggtgccgcgggcgctgagcacggccgaggtccgtcaggtggtggcggcgtttgcgaagagcgcgcggctagcggtgcaggctggggtggatgttatcgagatccatggggcgcatggctatctcatcaacgagttcctgagcccggtcacgaataagcggacggatgcgtacggcgggagctttgagaaccggacccggatcgtgcgcgaggttgcggcggctattcgtgcggtgattcccgaggggatgcccctgtttctgcgtatcagcgccacggagtggttggagggtcagccggtggccgcggagtcgggcagctgggatatgcagagctcgctggagctggtcaagaagctgcccgaatggggcattgacctggtggatgtcagctccgccgcgaaccacaaggaccagaagatcaacctgcacacggcctaccagacggacctggccgggcagattcgccaggccatccgagcggctggcgcgtcgactcttgtgggtgctgtaggtctgatcaccgattcggaacaggcgaggggactagttcagggagcggacgaggcgactgcagccgaggcaatgctgtcgggacctgaacccaaggcggatgccattctgatagcccgtcagttcctgcgcgagccagaatgggtgttttccacggcgagaaagttgggcgtgccggtgactgtcccggtgcagtttggcagggccatttag

SEQ ID NO: 8 - protein, 442 aa, Aspergillus fumigatus
MGSNAFRSPAVTKSSSTPYYTPANNGGAALHPDDPTTPTLFRPLQIRNVTLKNRIMVSPMCMYSCESDPSSPHVGALTNYHLAHLGHLALKGAGLVFIEATAVQPNGRISPNDSGLWQDGTTSEQFLGLKRVVEFMHAQGAKVGIQLAHAGRKASAVAPWLAAQAGKSSLKADESVGGWPADVVGPSGGEEHIFSPEEDAYWVPRALSTAEVRQVVAAFAKSARLAVQAGVDVIEIHGAHGYLINEFLSPVTNKRTDAYGGSFENRTRIVREVAAAIRAVIPEGMPLFLRISATEWLEGQPVAAESGSWDMQSSLELVKKLPEWGIDLVDVSSAANHKDQKINLHTAYQTDLAGQIRQAIRAAGASTLVGAVGLITDSEQARGLVQGADEATAAEAMLSGPEPKADAILIARQFLREPEWVFSTARKLGVPVTVPVQFGRAI

SEQ ID NO: 9 - DNA, 1269 bp, Aspergillus nidulans
atggctctccctgacgtcgaaaacacccccgccgccggcatcccctactttacaccagcacagaaccctcctgctggaacagctgccaacccgcaaaccagcggcaatgccgtccccaagctgtacacacctctgacggtgcgtggggtgaccttccacaacagacttggcctcgcgccgctctgccagtactccgcagaagacggccacatgacagactaccacatcgcgcacttgggaggtattgcccagcgcggccccggtctcatgatgatcgaggcaacctccgtctcacctgaaggcagaatcacgccgcaggacgtcggtttatggaaggactcgcagattgcgcccatgaagcgcgtcatcgacttcgtgcactcgcagtcccagaagattggcgtgcagattgcccacgccggccgcaaggcttcgaacatcgccccctggctcatgaacaagggcatcgtcgcgacggagaaggtcggtggctggccggatcgtgtgatcggcccgtccaccgtgcccttccacgagactttccccacccccaaggccatgaccaaggacgacatcgagcagttcaagcgcgactggtttgatgcgtgcaagcgggccattgccgctggcgcggacttcatcgagatccacaatgcccacgggtatcttctctcgtctttcctatcaccgtcttccaacacgcgcaccgacgagtacggcggctcctttgagaaccgcatccggctctctctcgaaatcgcccaggtcacccgtgacgccgtcggccccaacgttcctgtttttctccgtgtctccgcgacggactggatcgaggagaccctccccgaggaatcgtggaagctctctgactccgtccgcttcgccgaagccctcgctgcccagggcgctattgacctgatcgacgtctcttccggcggtgtccacgccgcgcagaagatcaagtccgggccggctttccaggctcccttcgctgtggctatcaagaaggccgttggcgataagctccttgttgcgacggtgggcacgatcacgaacggtaagcaggcgaacaagctgcttgaggaggagggattggatgttgcgcttgtgggacgtggtttccagaaggatcccggtctggcgtggactttcgcgcagcatcttgatgttgagattgcgatggcgagtcagattcggtggggattcacaaggcgcgggggcacgccttatatcgaccccaaagcttataaggagagcatctttgagtaa

SEQ ID NO: 10 - protein, 422 aa, Aspergillus nidulans
MALPDVENTPAAGIPYFTPAQNPPAGTAANPQTSGNAVPKLYTPLTVRGVTFHNRLGLAPLCQYSAEDGHMTDYHIAHLGGIAQRGPGLMMIEATSVSPEGRITPQDVGLWKDSQIAPMKRVIDFVHSQSQKIGVQIAHAGRKASNIAPWLMNKGIVATEKVGGWPDRVIGPSTVPFHETFPTPKAMTKDDIEQFKRDWFDACKRAIAAGADFIEIHNAHGYLLSSFLSPSSNTRTDEYGGSFENRIRLSLEIAQVTRDAVGPNVPVFLRVSATDWIEETLPEESWKLSDSVRFAEALAAQGAIDLIDVSSGGVHAAQKIKSGPAFQAPFAVAIKKAVGDKLLVATVGTITNGKQANKLLEEEGLDVALVGRGFQKDPGLAWTFAQHLDVEIAMASQIRWGFTRRGGTPYIDPKAYKESIFE

SEQ ID NO: 11 - DNA, 1299 bp, Candida albicans
atgacagttccataccaagtaaaaccatcagatgaaatcaaaggtgctcctgaggtttcctattacactccagaacagcctgttccggctggtactttttatccccaatcgtcagatgaagttgctcccaaaatttttcaacctttaaagattggtaagcttgctttgccaaacagaattggggtatctccaatgtgtcaatattctgctgattataattttgaagcaactccataccatttaatccattatggttcattagtgaatcgtgggccaggtatcaccattgttgaaagcacggctgtttctcctgagggtggattatcacctcatgatttaggaatctggaaggatgaacaagcagagaaattgaaaccaattgtcgattacgctcattctcaaaagcaattaattgccatccaattgggccatggtggtagaaaagcttctggtcagcccttatttttgcacttggaacaagttgcagataaatctgtcaatgggtttgccgacaaagcagttgctccttctgcattggcattcagaccaaatggtaatttacctgttcctaatgagttgaccaaagatgaaatcaaacgtgttgttaaggattttggtgctgctgctagaagagctgttgaaatcagtggctttgatgcagttgagattcatggtgctcatggttatttgattaatgagttctatagtcctatttcaaacaagagaacagatgaatacggtggcagttttgaaaatagaaccagatttttaaaggaagttatcgatagtgttaaatcaagtattccaaacgatgttccagtgtttttgagaatctctgctgctgaaaatagtcctgatccagaagcttggactattgaagattccaaaaaattagctgacattttagtagaaaagggtattgctttggttgatgtttcatctggtggtaacgattatagacaaccaccaagatctgggatcagtaaagagttgagagagccaatccatgttccgttgtctcgtgcaattaaacaacatgttggtgacaagttattggtcagttgcgttggtgggcttgaaaaagatcctgaattgctcaacaaatatttagaagaaggaacatttgatcttgctttgatcggtagaggatttttaagaaatccaggtttggtatgggagtttgccgataaacttggtgttagactccaccaggccttgcagttaggttggggtttctggcccaacaaacaacaaattgttgatttgattgaaagaacatctaaattagaagtaaattag

SEQ ID NO: 12 - protein, 432 aa, Candida albicans
MTVPYQVKPSDEIKGAPEVSYYTPEQPVPAGTFYPQSSDEVAPKIFQPLKIGKLALPNRIGVSPMCQYSADYNFEATPYHLIHYGSLVNRGPGITIVESTAVSPEGGLSPHDLGIWKDEQAEKLKPIVDYAHSQKQLIAIQLGHGGRKASGQPLFLHLEQVADKSVNGFADKAVAPSALAFRPNGNLPVPNELTKDEIKRVVKDFGAAARRAVEISGFDAVEIHGAHGYLINEFYSPISNKRTDEYGGSFENRTRFLKEVIDSVKSSIPNDVPVFLRISAAENSPDPEAWTIEDSKKLADILVEKGIALVDVSSGGNDYRQPPRSGISKELREPIHVPLSRAIKQHVGDKLLVSCVGGLEKDPELLNKYLEEGTFDLALIGRGFLRNPGLVWEFADKLGVRLHQALQLGWGFWPNKQQIVDLIERTSKLEVN

SEQ ID NO: 13 - DNA, 1110 bp, Candida albicans
atggaaaacaacaatactataccggcattatttcaacccataaagatcagtgactcgatcacattacctaatagaattggtgtttcaccaatgtgcatgtattcatcgtcaccaactgacaatcaagccactctgtttcattttgttcattatggatcatttgctgtacgtggaccagcattaatcattttagagagtatctttgtgtccgaaaattccggattatccattcatgatttaggtctttggaatgatgatcaagctcacagtttacggaaaattgttgattttattcatgatcaagacggaatttgctgtatacaattgaatcacgctgggcgaaagattgttgaaggggtaccattccaacaaatacaacatggttggcaagaacattgtgtggggccatctactgagccatttagtgattcacacaatacaccacgagaattgactgttaatgaaataaattcaattgtggaagactttgccaatgcagcttggcgggctgtggaaatctcaaaattcgatgccattgaaatacattgtgctaatggatgtttaatacaccaatttttaagtaaattgacaaacaagagagctgaccaatacgggggctcatttgaaaacagagttagatttcttttacaaataattgagaatataaaacgaaagatagaaacaccgattttcttaaagtttccaatgtcagataattgtagtgatccggaagcgtggtctacggaagatgcattgaagttggccgatcttgttattgatttaggagtaaaggtgatcgacgttacatcaggtggaaatgttgcgcattgcaaatctagatatctattaaatgacgacaaacaactaccttctcaagtgcccttggctcgtaaattgaaaagccacattagaaaccgatgtttgatcgcatgcagtggaggattagatcgagacatatttaaactcgatgagtttattgctaatggtgactttgatatagcattgataggtaaaggatttctcaaaaacactggattgatcagccgtattgctgaccaattgcaagcacaattcagaacagcacctcaatataagttggccttatcataa

SEQ ID NO: 14 - protein, 369 aa, Candida albicans
MENNNTIPALFQPIKISDSITLPNRIGVSPMCMYSSSPTDNQATLFHFVHYGSFAVRGPALIILESIFVSENSGLSIHDLGLWNDDQAHSLRKIVDFIHDQDGICCIQLNHAGRKIVEGVPFQQIQHGWQEHCVGPSTEPFSDSHNTPRELTVNEINSIVEDFANAAWRAVEISKFDAIEIHCANGCLIHQFLSKLTNKRADQYGGSFENRVRFLLQIIENIKRKIETPIFLKFPMSDNCSDPEAWSTEDALKLADLVIDLGVKVIDVTSGGNVAHCKSRYLLNDDKQLPSQVPLARKLKSHIRNRCLIACSGGLDRDIFKLDEFIANGDFDIALIGKGFLKNTGLISRIADQLQAQFRTAPQYKLALS

SEQ ID NO: 15 - DNA, 1305 bp, Neurospora crassa
atggccgacttcacccagaagaagacctcctcccccgcggccccgggtgttcccttctacaccccggcccaggtccccgccgccggcactcccctcccctccacccccggcgatgtccctactctcttcacccctctcaagatccgtggtgttgagctccagaaccgcttcgccgttgcgcccatgtgcacctactctgccgacgatggccacatgaccgactggcaccttgtccacctgggctccttcgccctccgcggtgtccccctcaccatcttcgaggccaccggcgtcctccccaacggccgcatcacccccgagtgctctggtctctggcaggactcccagattgcgcccctcaagcgcatcgtcgactacatccactcccagggccagaaggccggtatccagcttgcccacgccggccgcaaggcctccaccaaggccccctggcactaccagcgcggcaagagcgagcttgccggccccgagcagggtggctggcccgagaacgtctgggcccccagcgccatcagctacaacgaggagaccttccccttccccaaggagatgaccgtcgagcagatccacgagctcgtcgaggcctggaaggcgtctgcccagcgtgccctcaaggccggcttcgacctcattgagatccacgccgcccacggctacctcatttccgagttcttgagccccatctccaaccagcgtaccgaccagtacggtggctccttcgagaaccgcacccgcgttctccgcgagatcatctcggccgtccgctccgtcatccccgaggacatgcccctcttcgtccgtgtctccgccaccgagtggatggagtacaccggccagccctcgtgggacctccagcagaccattgagctcgccaagatcctccccgacctcggcgtcgacctcctcgacgtctcttccggcggcaacaacaaggaccagaagatcaacgtccacacctactaccagatcgacatggccgagcagatccgcgcggccgtgcacgaggccggcaagcagctcctcgtcggtgccgtcggcttggtcacctcggctgagatcgccaaggagaccgtccaggagaaggaggatggcagagtcaccatccagcgcgagaacggcgccaagactcgtgccgatatggtccttgttgccaggcagttcttgaaggagcccgagttcgtcctcactgtcgccgacgagttgggtgttgatgtcaaggcccctgttcagtacctccgtggtcctcttagcagcaggcccaagaagttgaccactgttccttaa

SEQ ID NO: 16 - protein, 434 aa, Neurospora crassa
MADFTQKKTSSPAAPGVPFYTPAQVPAAGTPLPSTPGDVPTLFTPLKIRGVELQNRFAVAPMCTYSADDGHMTDWHLVHLGSFALRGVPLTIFEATGVLPNGRITPECSGLWQDSQIAPLKRIVDYIHSQGQKAGIQLAHAGRKASTKAPWHYQRGKSELAGPEQGGWPENVWAPSAISYNEETFPFPKEMTVEQIHELVEAWKASAQRALKAGFDLIEIHAAHGYLISEFLSPISNQRTDQYGGSFENRTRVLREIISAVRSVIPEDMPLFVRVSATEWMEYTGQPSWDLQQTIELAKILPDLGVDLLDVSSGGNNKDQKINVHTYYQIDMAEQIRAAVHEAGKQLLVGAVGLVTSAEIAKETVQEKEDGRVTIQRENGAKTRADMVLVARQFLKEPEFVLTVADELGVDVKAPVQYLRGPLSSRPKKLTTVP

SEQ ID NO: 17 - DNA, 1476 bp, Neurospora crassa
atggctacttccactacctccgacctcaaactctcccaacccctcaccctccccaatggccttaccctccccaaccgcctcgtcaaagccgccatggccgaacaaatgggcttcggcaaccacctgcccaaccccgaactcgccgccgtctacgccacctgggcccgcggcgactggggcctgattctcaccggcaacgtccaagtcgaccacgcgcacaagggcgacgcccacgacatcagccccaaccaccccggcaccacgcccgagcagaccgtcacggccttcaaggcctgggcggacgccgcgcgcctgaatggccagtccaaaacgcctgtggtcgtgcagatcaaccaccctggtcgccagagtccgatgggcgcgggcacgcggggactgtgggagaaggcggtggcgccctcgccggtgccgttggtgttgggagaggcgtttgtgcctcgcttgttgtcgaaagtgcttttcggcacgccgcgggagctgacggttgcggagatcaaggatatcgtgcaaaagtttgcggtgacggcgaggatcacggccgaggccgggttcaatggcgtggagatccatgcggcgcatggatacctgttggcgcagttcttgagcaagaagacaaacaggcgcggggatgagtatggcgggtcggctgagaacagggcgaggattgttggggagattattaaggagtgcaggaggcaggtgactgaggcggtgggtgaagaggaggcgaagaagtttgtggtgggaatcaagctgaacagtgcggattggcaggcgggacgcgatggaaaggaggaggaggagacggatacggcggaggaggtgttgaagcagattgagctttttgagcagtgggggatcgactttgtcgaggttagcggtggcagttatgaggatcctcaggtaagttttggtgttgtttgagggatggggcaaggggttgtctgtcgtgaacaacaaaaggggcacggaacaaatgctaacgccatacagatggccaacggtcccaagcccgaaaagtccgaacgcaccatggcccgcgaggccttcttcctcgagttcgccaagatcatccgcaccaagttccccaagcttcctctcatggtcaccggcggcttccgcactcgtcagggcatggaggccgctttggaatccgatgattgcgacatgatcggtatcggacgcccggccatcatcaacccttcgcttcccgccaacttgatcctcaacccggaggtgccggatgcggatgcccgcttgttcgacaagaagagggctgagccgcactggatcgttgagaagttgggcatgaagtccattgttggtgctggtgttgaggtggtacgtcacgttccaaccccatttgcttcattgtgtttccgagtatgtcatgctgacttggttcttttctagacgtggtatgtgagcgagctcaagaagctggccaagttttag

SEQ ID NO: 18 - DNA, 1314 bp, Neurospora crassa
atggctacttccactacctccgacctcaaactctcccaacccctcaccctccccaatggccttaccctccccaaccgcctcgtcaaagccgccatggccgaacaaatgggcttcggcaaccacctgcccaaccccgaactcgccgccgtctacgccacctgggcccgcggcgactggggcctgattctcaccggcaacgtccaagtcgaccacgcgcacaagggcgacgcccacgacatcagccccaaccaccccggcaccacgcccgagcagaccgtcacggccttcaaggcctgggcggacgccgcgcgcctgaatggccagtccaaaacgcctgtggtcgtgcagatcaaccaccctggtcgccagagtccgatgggcgcgggcacgcggggactgtgggagaaggcggtggcgccctcgccggtgccgttggtgttgggagaggcgtttgtgcctcgcttgttgtcgaaagtgcttttcggcacgccgcgggagctgacggttgcggagatcaaggatatcgtgcaaaagtttgcggtgacggcgaggatcacggccgaggccgggttcaatggcgtggagatccatgcggcgcatggatacctgttggcgcagttcttgagcaagaagacaaacaggcgcggggatgagtatggcgggtcggctgagaacagggcgaggattgttggggagattattaaggagtgcaggaggcaggtgactgaggcggtgggtgaagaggaggcgaagaagtttgtggtgggaatcaagctgaacagtgcggattggcaggcgggacgcgatggaaaggaggaggaggagacggatacggcggaggaggtgttgaagcagattgagctttttgagcagtgggggatcgactttgtcgaggttagcggtggcagttatgaggatcctcagatggccaacggtcccaagcccgaaaagtccgaacgcaccatggcccgcgaggccttcttcctcgagttcgccaagatcatccgcaccaagttccccaagcttcctctcatggtcaccggcggcttccgcactcgtcagggcatggaggccgctttggaatccgatgattgcgacatgatcggtatcggacgcccggccatcatcaacccttcgcttcccgccaacttgatcctcaacccggaggtgccggatgcggatgcccgcttgttcgacaagaagagggctgagccgcactggatcgttgagaagttgggcatgaagtccattgttggtgctggtgttgaggtgacgtggtatgtgagcgagctcaagaagctggccaagttttag

SEQ ID NO: 19 - protein, 437 aa, Neurospora crassa
MATSTTSDLKLSQPLTLPNGLTLPNRLVKAAMAEQMGFGNHLPNPELAAVYATWARGDWGLILTGNVQVDHAHKGDAHDISPNHPGTTPEQTVTAFKAWADAARLNGQSKTPVVVQINHPGRQSPMGAGTRGLWEKAVAPSPVPLVLGEAFVPRLLSKVLFGTPRELTVAEIKDIVQKFAVTARITAEAGFNGVEIHAAHGYLLAQFLSKKTNRRGDEYGGSAENRARIVGEIIKECRRQVTEAVGEEEAKKFVVGIKLNSADWQAGRDGKEEEETDTAEEVLKQIELFEQWGIDFVEVSGGSYEDPQMANGPKPEKSERTMAREAFFLEFAKIIRTKFPKLPLMVTGGFRTRQGMEAALESDDCDMIGIGRPAIINPSLPANLILNPEVPDADARLFDKKRAEPHWIVEKLGMKSIVGAGVEVTWYVSELKKLAKF

SEQ ID NO: 20 - DNA, 1412 bp, Magnaporthe grisea
atgtcggcagaaaagaagactttgagcaaaccggccgccggggtgccttactacaccccagcccaggagccgccggcagggacccctttgcagcagcaggacgccatcccaacgctgttcaagcctctgaagatccgtggcgtcgagctctccaaccgctttggcgtctcgcccatgtgcacctactcagccgacgatggccacctgaccgacttccacttggtgcacctgggccagttcgccctgcacggcacggccctgaccattgtcgaggccacatccgtcacgcccaacggacgcatctcgcccgaggacagcggcctgtggcaagacagccagatcgctcctctgcgccgcatcgtcgactacgtgcacagccagggccaaaagatcgccatccaactggctcatgccggccgcaaggccagcacaaaggccccctggcacgactccttcacccccagcggcgagtataagccgagagagggcttacaggtcgtcggacccgagtatggcggctggcctgatgacgtctgggccccgagcgccatcccgttctcggaggactttccgaaccccaaggagatgaccgttgaggagattgagggactcgtcaccagctttgtggacgctgccaagcgtgccatcgaggccggcgtcgacattattgagattcacggcgctcacggttacctgatcaccgagttcctttcgccgctatcaaacgtaagtggagatactttgtgtggggctgtgcgcatactccctcgggtgtgacttctattaacattttatttcctggcacgcagaaacggacagacaagtacggcggcagctttgagaaccgcacccgggtcctgatcgatattatcaaggccgtccgggcagtgattcccgaggagatgccactcttcgtccgaatctccgcgaccgaatggatggagtacgccggcgagcctagctgggacctcgagcagagcacacagcttgccaagctcctcccggacctgggtgtcgacctgctcgacgtcagctcgggcggaaactcggtggcccaaaagatcgagctcacgccgtactaccagatcgacctggcagccaagatccgcgaggccgtcggcgataggttgctcataggcgcggtcggcaacatcaacacggctgacattgcgcgcgatgtcgtggatgagcagggcgccgagaaggtggccgaggccaagcagacgcatgacaccatcgaggtcgtgagcgaatcacatggcggcaagaccaaggcggatctggtcctcattgctcgccagttcctgcgcgagcctgagtttgtgctgaggacggcgcataaccttggggtcaatgtgcagtggcctcaccaataccacagagcagtgtggcgcaagggtgcaaggatttga

SEQ ID NO: 21 - DNA, 1329 bp, Magnaporthe grisea
atgtcggcagaaaagaagactttgagcaaaccggccgccggggtgccttactacaccccagcccaggagccgccggcagggacccctttgcagcagcaggacgccatcccaacgctgttcaagcctctgaagatccgtggcgtcgagctctccaaccgctttggcgtctcgcccatgtgcacctactcagccgacgatggccacctgaccgacttccacttggtgcacctgggccagttcgccctgcacggcacggccctgaccattgtcgaggccacatccgtcacgcccaacggacgcatctcgcccgaggacagcggcctgtggcaagacagccagatcgctcctctgcgccgcatcgtcgactacgtgcacagccagggccaaaagatcgccatccaactggctcatgccggccgcaaggccagcacaaaggccccctggcacgactccttcacccccagcggcgagtataagccgagagagggcttacaggtcgtcggacccgagtatggcggctggcctgatgacgtctgggccccgagcgccatcccgttctcggaggactttccgaaccccaaggagatgaccgttgaggagattgagggactcgtcaccagctttgtggacgctgccaagcgtgccatcgaggccggcgtcgacattattgagattcacggcgctcacggttacctgatcaccgagttcctttcgccgctatcaaacaaacggacagacaagtacggcggcagctttgagaaccgcacccgggtcctgatcgatattatcaaggccgtccgggcagtgattcccgaggagatgccactcttcgtccgaatctccgcgaccgaatggatggagtacgccggcgagcctagctgggacctcgagcagagcacacagcttgccaagctcctcccggacctgggtgtcgacctgctcgacgtcagctcgggcggaaactcggtggcccaaaagatcgagctcacgccgtactaccagatcgacctggcagccaagatccgcgaggccgtcggcgataggttgctcataggcgcggtcggcaacatcaacacggctgacattgcgcgcgatgtcgtggatgagcagggcgccgagaaggtggccgaggccaagcagacgcatgacaccatcgaggtcgtgagcgaatcacatggcggcaagaccaaggcggatctggtcctcattgctcgccagttcctgcgcgagcctgagtttgtgctgaggacggcgcataaccttggggtcaatgtgcagtggcctcaccaataccacagagcagtgtggcgcaagggtgcaaggatttga

SEQ ID NO: 22 - protein, 442 aa, Magnaporthe grisea
MSAEKKTLSKPAAGVPYYTPAQEPPAGTPLQQQDAIPTLFKPLKIRGVELSNRFGVSPMCTYSADDGHLTDFHLVHLGQFALHGTALTIVEATSVTPNGRISPEDSGLWQDSQIAPLRRIVDYVHSQGQKIAIQLAHAGRKASTKAPWHDSFTPSGEYKPREGLQVVGPEYGGWPDDVWAPSAIPFSEDFPNPKEMTVEEIEGLVTSFVDAAKRAIEAGVDIIEIHGAHGYLITEFLSPLSNKRTDKYGGSFENRTRVLIDIIKAVRAVIPEEMPLFVRISATEWMEYAGEPSWDLEQSTQLAKLLPDLGVDLLDVSSGGNSVAQKIELTPYYQIDLAAKIREAVGDRLLIGAVGNINTADIARDVVDEQGAEKVAEAKQTHDTIEVVSESHGGKTKADLVLIARQFLREPEFVLRTAHNLGVNVQWPHQYHRAVWRKGARI

SEQ ID NO: 23 - DNA, 1188 bp, Schizosaccharomyces pombe
atgactattgttaatgaaggagccgaaaatgttggttattttacacctgcgcaaaaaataccagctggagcggcgataggtgtaccgcaaacaaaattatttactcctcttaaaattagaggagtggagttccataacagaatgtttgtttcgccgatgtgcacttattccgctgaccaagaagggcatttgacagattttcacctagtacatcttggagcgatgggaatgcgtgggcctggccttgtaatggtagaagcgacagcggtttccccagagggacgaatttcacctaatgattcaggattatggatggagtcgcaaatgaagccgttacgaagaattgttgaatttgctcattcgcaaaatcaaaaaattgggattcaattggcgcatgctggtagaaaggctagcaccactgctccttatcgaggatacacagttgcgactgaagctcaaggtgggtgggagaatgatgtttatggaccaaatgaagacaggtgggacgaaaaccacgctcaacctcataagttaactgaaaagcaatatgatgaattagtggataagtttgttgttgctgcgaagcgtgcagttgaaataggttttgatgtaattgaaattcatggcgctcatggttatcttatatcgtcaacagttagtcctgccactaatgaccgcaatgacaagtatggtgggacatttgagaaacgtattttgtttcctatggaagttgtccattctgttcgtaaagcaattccagatagtatgcccttgttttatagagtaacggctacagattggttgcccaaaggacaaggatgggagatagaagatacagttgcattagcagcgaggcttcgcgatggtggtgttgacttgatagatgttagctctggtggtaatcacaaggatcaaagaattgaggtgaaggattgctatcaagttccttttgcggaaaagattaaggatcaagtgaatggaatactacttggcgctgtcggaatgatcagggatggtcttacggcgaatgaaatcctagaaagtggaaaagctgatgttacttttgtcgcaagggagttcttaaggaacccgtcgttggtgctagacagcgcgaaccagttgggtgaaaatgttgcatggccagttcagtatgactatgcagttaagggacacagaaagttacgttga

SEQ ID NO: 24 - protein, 407 aa, Schizosaccharomyces pombe
MTIVNEGAENVGYFTPAQKIPAGAAIGVPQTKLFTPLKIRGVEFHFTNRMFVSPMCTYSADQEGHLTDFHLVHLGAMGMRGPGLVMVEATAVSPEGRISPNDSGLWFTMESQMKPLRRIVEFAHSQNQKIGIQLAHAGRKASTTAPYRGYTVATEAQGGWENDVYGPFTNEDRWDENHAQPHKLTEKQYDELVDKFVVAAKRAVEIGFDVIEIHGAHGYLISSTVSPAFTTNDRNDKYGGTFEKRILFPMEVVHSVRKAIPDSMPLFYRVTATDWLPKGQGWEIEDTVAFTLAARLRDGGVDLIDVSSGGNHKDQRIEVKDCYQVPFAEKIKDQVNGILLGAVGMIRDGLFTTANEILESGKADVTFVAREFLRNPSLVLDSANQLGENVAWPVQYDYAVKGHRKLR

SEQ ID NO: 25 - DNA, 777 bp, Colletotrichium trifolii
cgaaacctcgacccaaacaaacagctgaccctctccttgacaacaaagccggccatcctcgccgacgattgcctctacccccgcatagtcacactcgcacgtccgttctcccaccgtcaaacagacagcatgacgggcaccgcgaacaaggccgcccccggtgtgccgttttacaccccggcccaggagcctcccgcgggaacgccagtcgacgccagcacggctccgacgctcttcaagcccctccgcatccgcgacctcaccatcaacaaccgcatctgggtcagccccatgtgccagtactccgccgacaatggccacgcgaccgactaccacctcgtccacctgggccagttcgccctgcacggcgccgccctgtccatggtcgaggccaccgccgtcgaggctcgtggccgcatctcccccgaggatgtcggtttgtggcaggactcgcagattgcgccgctgaagcgcatcgtcgactttatccactcgcagaaccaggtcgcggccatccagctcgcccacgccggtcgcaaggctagcaccctggcaccgtggatcaccgaggctcgcggcaaggcgctggctcaggagagcgagaacggctggcccgacgacgttgtggctcccagcgcgattccttacaccaaggactgggccacaccgcgtgagttgactaccgaggrrgtcgagggtctgggtgaagaagttcgccgagtcggccaagaggtcaaatcgagctggttttgacgtcattgagatccacgccgctca

SEQ ID NO: 26 - DNA, 645 bp, Colletotrichium trifolii
atgacgggcaccgcgaacaaggccgcccccggtgtgccgttttacaccccggcccaggagcctcccgcgggaacgccagtcgacgccagcacggctccgacgctcttcaagcccctccgcatccgcgacctcaccatcaacaaccgcatctgggtcagccccatgtgccagtactccgccgacaatggccacgcgaccgactaccacctcgtccacctgggccagttcgccctgcacggcgccgccctgtccatggtcgaggccaccgccgtcgaggctcgtggccgcatctcccccgaggatgtcggtttgtggcaggactcgcagattgcgccgctgaagcgcatcgtcgactttatccactcgcagaaccaggtcgcggccatccagctcgcccacgccggtcgcaaggctagcaccctggcaccgtggatcaccgaggctcgcggcaaggcgctggctcaggagagcgagaacggctggcccgacgacgttgtggctcccagcgcgattccttacaccaaggactgggccacaccgcgtgagttgactaccgaggrgtcgagggtctgggtgaagaagttcgccgagtcggccaagaggtcaaatcgagctggttttgacgtcattgagatccacgccgct

SEQ ID NO: 27 - protein, 215 aa, Colletotrichium trifolii
  misc_feature (187)..(187): Xaa can be any naturally occurring amino acid
MTGTANKAAPGVPFYTPAQEPPAGTPVDASTAPTLFKPLRIRDLTINNRIWVSPMCQYSADNGHATDYHLVHLGQFALHGAALSMVEATAVEARGRISPEDVGLWQDSQIAPLKRIVDFIHSQNQVAAIQLAHAGRKASTLAPWITEARGKALAQESENGWPDDVVAPSAIPYTKDWATPRELTTEXSRVWVKKFAESAKRSNRAGFDVIEIHAA

SEQ ID NO: 28 - DNA, 803 bp, Fusarium sporotrichioides
gaactgctgtagatgtggttgaattggtatattagaccggagtactctatatgcgagagactatacattgaagttgccaacgttcttccagattgattaatcatggcttacgagataatcgacaacgttgcggctgaaggggttccatattacacaccggctcaagacccgccagctggtacgcagacaagcggctcaacgaagctattcacacccatcaccatccgcggcgtcacattcccaaaccgcctcttccttgcccctctctgccaatactccgccaaagatggttatgccactgattggcacttgactcacctcgggggaataatccaaagaggccccggattgtccatggtggaggctaccgctgtacaaaaccacggtcgcatcacacctcaggatgttggtctgtgggaagacggccagatcgagcctctgaagcgcatcaccactttcgcgcacagtcagagccagaaaattggtatccagctgtcgcatgcgggtcgcaaggccagttgcgtatctccctggctaagcgtaaatgctgtcgcggcggaagaagtgggtggctggccagacaatatcgttgctccctcggccatcgcacaagaaaatggtgtgaacccagttcccaaggctttcacgaaggaggatatagagcaactcaagagcgactacgtggaagcggcaaaacgagccatccatgctggtttcgatgttatcgaaattcatgcagctcatggatatctactgcatcaattcttgagtccggtaagcaatcaaagaaccgacgagtatgg

SEQ ID NO: 29 - DNA, 701 bp, Fusarium sporotrichioides
atggcttacgagataatcgacaacgttgcggctgaaggggttccatattacacaccggctcaagacccgccagctggtacgcagacaagcggctcaacgaagctattcacacccatcaccatccgcggcgtcacattcccaaaccgcctcttccttgcccctctctgccaatactccgccaaagatggttatgccactgattggcacttgactcacctcgggggaataatccaaagaggccccggattgtccatggtggaggctaccgctgtacaaaaccacggtcgcatcacacctcaggatgttggtctgtgggaagacggccagatcgagcctctgaagcgcatcaccactttcgcgcacagtcagagccagaaaattggtatccagctgtcgcatgcgggtcgcaaggccagttgcgtatctccctggctaagcgtaaatgctgtcgcggcggaagaagtgggtggctggccagacaatatcgttgctccctcggccatcgcacaagaaaatggtgtgaacccagttcccaaggctttcacgaaggaggatatagagcaactcaagagcgactacgtggaagcggcaaaacgagccatccatgctggtttcgatgttatcgaaattcatgcagctcatggatatctactgcatcaattcttgagtccggtaagcaatcaaagaaccgacgagtatgg

SEQ ID NO: 30 - protein, 233 aa, Fusarium sporotrichioides
MAYEIIDNVAAEGVPYYTPAQDPPAGTQTSGSTKLFTPITIRGVTFPNRLFLAPLCQYSAKDGYATDWHLTHLGGIIQRGPGLSMVEATAVQNHGRITPQDVGLWEDGQIEPLKRITTFAHSQSQKIGIQLSHAGRKASCVSPWLSVNAVAAEEVGGWPDNIVAPSAIAQENGVNPVPKAFTKEDIEQLKSDYVEAAKRAIHAGFDVIEIHAAHGYLLHQFLSPVSNQRTDEY

SEQ ID NO: 31 - DNA, 631 bp, Fusarium sporotrichioides
tttggatggtataataataattctatttgtgaaacatacggggctggtcttgatcaagaacggtccatctatggtctataaagaactcttgttcactttctttccacgtcccttgaagctccaatcaatctggttcgccatcttgacctccacgccaagctcgttagcaaaagctcgaaccagaccaggattctgttggaaccaacgtccagccctcacaatgtcgataccagattgcaaaacctcttcagcaagatgtccagtcttgattccacctactgctgaaacaagtacactatcgccaacagccttctttacctgtttggcgaggtctacctggtaagcaggaccggacttgatggcgatggcggacttaggatggataccgcctgagctgacgtccaccaagtctactccatgcttgggcaagatacgcgcgagttgacaagtctgctcgactgtccagctttcaggaaactcgtctttgaattgagagtcaaactcgaaccaatcagttgcactgacacgaacgaggacaggtgtagtttcggggatggcagcgcggatgaggtcaaggatttccaagacaactctgatacggttctcgaaactgccaccatactcgtcggtt

SEQ ID NO: 32 - DNA, 556 bp, Fusarium sporotrichioides
aaccgacgagtatggtggcagtttcgagaaccgtatcagagttgtcttggaaatccttgacctcatccgcgctgccatccccgaaactacacctgtcctcgttcgtgtcagtgcaactgattggttcgagtttgactctcaattcaaagacgagtttcctgaaagctggacagtcgagcagacttgtcaactcgcgcgtatcttgcccaagcatggagtagacttggtggacgtcagctcaggcggtatccatcctaagtccgccatcgccatcaagtccggtcctgcttaccaggtagacctcgccaaacaggtaaagaaggctgttggcgatagtgtacttgtttcagcagtaggtggaatcaagactggacatcttgctgaagaggttttgcaatctggtatcgacattgtgagggctggacgttggttccaacagaatcctggtctggttcgagcttttgctaacgagcttggcgtggaggtcaagatggcgaaccagattgattggagcttcaagggacgtggaaagaaagtgaacaagagttctttatag

SEQ ID NO: 33 - protein, 184 aa, Fusarium sporotrichioides
TDEYGGSFENRIRVVLEILDLIRAAIPETTPVLVRVSATDWFEFDSQFKDEFPESWTVEQTCQLARILPKHGVDLVDVSSGGIHPKSAIAIKSGPAYQVDLAKQVKKAVGDSVLVSAVGGIKTGHLAEEVLQSGIDIVRAGRWFQQNPGLVRAFANELGVEVKMANQIDWSFKGRGKKVNKSSL

SEQ ID NO: 34 - DNA, 657 bp, Fusarium sporotrichioides
aggaagttgcatgtcacttgtagtgacagggcgtcgtgtaaattttataaatacctatacttgtttgttcacttctatgctactcatatcaatccgagaagatcaaacagtcccctatacacacttgtcaagacctatctattatttcaaaaatcagcaatatggctgagacaatgcctaagtgtgaggcaaatggccatcacaaaatcatcatcaataaggaagctccgaatgttcctttctatactccagtgcaagatccaccagcaggaacgtcttacgatgttcagcctgaaggaagcctattctctcttattaaaataagaaacctgactcttcaaaaccggatttttgtctccccaatgtgtcaatattcagcaaaggatggtgtcatgaccccctggcacaaacaacacctgggcagcttcgcagcacgaggtccgggtctcattgtcacagaagtcaacgcagtttcaccagagggacgaatcagtcctgaggatgcaggcatctacgatgatgggcagcttggacctctccgggatattgtggactttgtacacagccagggcgccaagattgctattcagataggtcatgctgggagaaaagcgagcacagtcgtaccgtggctggaccgcaagaacactgctttta

SEQ ID NO: 35 - protein, 161 aa, Fusarium sporotrichioides
MPKCEANGHHKIIINKEAPNVPFYTPVQDPPAGTSYDVQPEGSLFSLIKIRNLTLQNRIFVSPMCQYSAKDGVMTPWHKQHLGSFAARGPGLIVTEVNAVSPEGRISPEDAGIYDDGQLGPLRDIVDFVHSQGAKIAIQIGHAGRKASTVVPWLDRKNTAF

SEQ ID NO: 36 - DNA, 744 bp, Fusarium graminearum
gcacgagggattattgacaacatcgcggctgaaggggctccctactacacgcctgctcaagacyctccagcaggcacacagaccagcggctcaaccaaggttttcacacbcatcaccatccgaggcgtcacattcccaaaccgtctctttcttgcccctctctgtcaatactccgccaaagatggatatgctactgattggcacttgactcatctcggaggcattatccaacgaggcccgggactgtccatggtagaggccaccgctgttcaaaaccacggtcgcatcacgcctcaggacgttggtctctgggaagatggacaaatcgagccctttgaagcgcatcactacttttgcccacagccaaagcwcagaagattggtattcagctctcgcacgctggtcgtaaggctagttgtgtatctccgtggttgagcatcaacgctgttgccgctaaggaagtcggtggctggccagacaacattgttgctccttctgccatcgcacaagaagctggcgtgaaccctgttcccaaggccttcaccaaggaggatatcgaggaactcaagaatgactttctggctgcagcmaaacgagccawccgcgctggttttgatgtcatcgagatccatgcagctcatggatacktgcttcaccagttcttgagtccagtcagtaaccaaagaaccgatgagtatggtggcagcttcgagaaccgtatcagagtcgtcttggagatcattg

SEQ ID NO: 37 - DNA, 742 bp, Fusarium graminearum
gcacgagggattattgacaacatcgcggctgaaggggctccctactacacgcctgctcaagacyctccagcaggcacacagaccagcggctcaaccaaggttttcacacbcatcaccatccgaggcgtcacattcccaaaccgtctctttcttgcccctctctgtcaatactccgccaaagatggatatgctactgattggcacttgactcatctcggaggcattatccaacgaggcccgggactgtccatggtagaggccaccgctgttcaaaaccacggtcgcatcacgcctcaggacgttggtctctgggaagatggacaaatcgagcccttgaagcgcatcactacttttgcccacagccaaagccagaagattggtattcagctctcgcacgctggtcgtaaggctagttgtgtatctccgtggttgagcatcaacgctgttgccgctaaggaagtcggtggctggccagacaacattgttgctccttctgccatcgcacaagaagctggcgtgaaccctgttcccaaggccttcaccaaggaggatatcgaggaactcaagaatgactttctggctgcagcmaaacgagccawccgcgctggttttgatgtcatcgagatccatgcagctcatggatacktgcttcaccagttcttgagtccagtcagtaaccaaagaaccgatgagtatggtggcagcttcgagaaccgtatcagagtcgtcttggagatcattg

SEQ ID NO: 38 - protein, 247 aa, Fusarium graminearum
  misc_feature (22)..(22): Xaa can be any naturally occurring amino acid
ARGIIDNIAAEGAPYYTPAQDXPAGTQTSGSTKVFTXITIRGVTFPNRLFLAPLCQYSAKDGYATDWHLTHLGGIIQRGPGLSMVEATAVQNHGRITPQDVGLWEDGQIEPLKRITTFAHSQSQKIGIQLSHAGRKASCVSPWLSINAVAAKEVGGWPDNIVAPSAIAQEAGVNPVPKAFTKEDIEELKNDFLAAXKRAXRAGFDVIEIHAAHGYXLHQFLSPVSNQRTDEYGGSFENRIRVVLEII

SEQ ID NO: 39 - DNA, 647 bp, Mycosphaerella graminicola
cctcaagatccgaggtcttaccctccagaaccgtattatgttgagggggctctgccagtactctgctcccgacggacactacacaatgtggcatcacacccacatgggcggcatcatccaacgcggtcccggactcacctgcgttgaagccacagccgtgactcctcaaggtcgcatcacgcctgaagacgtcggtatctggcaagattctcagatcgagcctcttgccaaggtcgtcgagtttgcccactcccagaaccagaagatcatgattcagttggcgcatgcgggccgcaaagcgagcactgtggcaccatggttaagcggcggcgatgttgctggtgaggacgtcaacggatggccacaggatgtctgggcgcccagtgcgattccatggaacgagaagcacgctgtcccaaaggagatgtcgttggatgatatcgaggctttcaagaaggcgtttggagaggcggtcaagcgggcattgaaggctggatttgatgttattgagattcacaatgctcacggatacctcctccacgaattcatctgcctgagagcaacaccaggaccgacaagtacgggcggaagctgggaaaaccgcactcgtctgacaatggaaagtcgtcgaccttgtccgcagcatt

SEQ ID NO: 40 - protein, 215 aa, Mycosphaerella graminicola
LKIRGLTLQNRIMLRGLCQYSAPDGHYTMWHHTHMGGIIQRGPGLTCVEATAVTPQGRITPEDVGIWQDSQIEPLAKVVEFAHSQNQKIMIQLAHAGRKASTVAPWLSGGDVAGEDVNGWPQDVWAPSAIPWNEKHAVPKEMSLDDIEAFKKAFGEAVKRALKAGFDVIEIHNAHGYLLHEFICLRATPGPTSTGGSWENRTRLTMESRRPCPQH

SEQ ID NO: 41 - DNA, 560 bp, Mycosphaerella graminicola
gactgccgagtaaacgcgccggcaaggaggcgggaggatggccggaggatgttgtgggtccgtcgggtggggaggactttacgtgggatgagaggtcctcgagcgaccctagtggaggctactatgcgccgagagagttgtcggtcagagagatcaaggagatggtccaagactgggcgacagcagcgaaaagggcggtgaaagcgggcgtggatgtaatcgaaatccacggcgcgcatgggtacctcatccacgaattcctctcacccattaccaaccgccggacagattcttacggcggttctttcgaaaaccgtacccgtctactcattgaaatcgtaacagccgtccgagccgcgatgccctccagcatgcctctcttcctccgcctctcctctacagaatggatggaagataccgacatcggcaagaagttcggaagctgggatgtcgaaagcacgatcaagatctccaaaatcctggccgacttgggcgttgatctcctcgacgtgtcttccggtgggaatcatcctcagcagaaaatcaacatgttcaacacc

SEQ ID NO: 42 - protein, 186 aa, Mycosphaerella graminicola
LPSKRAGKEAGGWPEDVVGPSGGEDFTWDERSSSDPSGGYYAPRELSVREIKEMVQDWATAAKRAVKAGVDVIEIHGAHGYLIHEFLSPITNRRTDSYGGSFENRTRLLIEIVTAVRAAMPSSMPLFLRLSSTEWMEDTDIGKKFGSWDVESTIKISKILADLGVDLLDVSSGGNHPQQKINMFNT

SEQ ID NO: 43 - DNA, 1254 bp, Magnaporthe grisea
atgtccccaccacgcttcgaagcggcccctgccgacccctcaccgctcggcacgccgctcaaataccccgtctcggggcggtcggcgcccaaccggttcctcaacgcggccatgtcggagggcctggcgacgtttgacgaggcggacccgtccaagcgcggcatcccgacggagcagctggtgcagctgtaccggcgctggggccagggcgagtggggccagatccagacgggcaacgtcatgatcgacccggagcacctcgaggccccgggcaacatggtggtgccgcgcgacgccgagccctcgggcgagcgcttcgacatgttttccaagctcgccgccgccgccaaggagcacggcagcctcatcgtcgcgcaggtcggacaccccggtcgccaggcccgcggcagcgtccagcagcaccccattagcgccagcgacgtgcagcttaagcaggagatgtttgggtcaaagtttggcgtgcccaggcccgctaccaaggaggatattaaggcggtgattgagggttttgcccacacggccgagtaccttgaaaaggccggtttcgacggtatcgaattgcacgccgcccacggttacctgctggcccaattcctgtccgaaacaaccaaccagcgcaccgacgagtacggcggcagcctcgaaaaccgcatgcggctaatcctcgaggtcacggccgaggtccgcaggcggacgagcaagaatttcatcctcggcatcaaaattaacagcgtcgagttccaggagaagggtttcaagccagaggaggcggtgcagttgtgcgaggccctcgaggccgcgggcatggattttgtcgagacgagcggcggcacctatgagagttttggttttgcgcaccgcaaggagtccagccgcaagcgggagaactattttatcgagttcgccgaggtcatccgcaaggccgtcaagcacatggtggtctacaccaccggcggcttcaagacggtgggcgccatggtcgacgcgctgcagggcgtcgatgggataggcatcgggcgcgcagccggttcggagccggacctcgccaaggacatcatcgcgggcaaggtgtccagcattatcaaatacgccatgggggaggacgagtttgtgctgcagttgactgcctgctcggcgcaaataaggctgatggccaagggcgaggagccgtttgacatctcaaacgccgacgaggtggcgcgggtgacgcagttgatggcggagggcaaggtg

SEQ ID NO: 44 - protein, 418 aa, Magnaporthe grisea
MSPPRFEAAPADPSPLGTPLKYPVSGRSAPNRFLNAAMSEGLATFDEADPSKRGIPTEQLVQLYRRWGQGEWGQIQTGNVMIDPEHLEAPGNMVVPRDAEPSGERFDMFSKLAAAAKEHGSLIVAQVGHPGRQARGSVQQHPISASDVQLKQEMFGSKFGVPRPATKEDIKAVIEGFAHTAEYLEKAGFDGIELHAAHGYLLAQFLSETTNQRTDEYGGSLENRMRLILEVTAEVRRRTSKNFILGIKINSVEFQEKGFKPEEAVQLCEALEAAGMDFVETSGGTYESFGFAHRKESSRKRENYFIEFAEVIRKAVKHMVVYTTGGFKTVGAMVDALQGVDGIGIGRAAGSEPDLAKDIIAGKVSSIIKYAMGEDEFVLQLTACSAQIRLMAKGEEPFDISNADEVARVTQLMAEGKV

SEQ ID NO: 45 - DNA, 690 bp, Aspergillus fumigatus
agcttagacctacagagagcattgctactgtaagttgtatttcgccttctcgcatagaacaaaatataactgatggtgtaggtataaaactagcatcctcttccacctttcagatccccctgacaagcaccttatggctttcgatggaaacagctattccttctactggtaaaaataggataccagaggctacaatcaatacaccctcgatagaggctgtcgaatgtggccaactggcaacgctgcggttagtcatcgtcggagactttctgggattcattttcttccgagtctccgcctgcttattaaggcatcaatctggatgctccactgtggtacatccaattttcgatttttcttcggcagaggcaaggattccactggttcagtctaggcatttagaagatcaaagctgtcctgtacctccgtacctgggtgttcgacgtcattgccacgtttcgacccaagggcagacgccatgtcgccgagcgatcgccgcgatatgcctcgaatttgcgccattcggcatccagtttccagtgcccttccccgaatgactgtctccactattcggcaagattgtaaatcaagcctgaagaagcggagcattcttggaagtcgtatgttctactgattctgtgcctggcgcagacgggtatataataaagatcacgcaccgaggagttctta

SEQ ID NO: 46 - DNA, 19 bp, Artificial sequence
  Primer
gttcgacgtcattgccacg

SEQ ID NO: 47 - DNA, 19 bp, Artificial sequence
  primer
ccttgatcgttgctgagcg

SEQ ID NO: 48 - DNA, 19 bp, Artificial sequence
  primer
atgactgtcgccgatatcg

SEQ ID NO: 49 - DNA, 22 bp, Artificial sequence
  primer
ctatacatcgaaaatagactgc

SEQ ID NO: 50 - DNA, 26 bp, Artificial sequence
  primer
ccgtcctgggcggagtattggcagag

SEQ ID NO: 51 - DNA, 31 bp, Artificial sequence
  primer
gcgaatcagattctagaggagcaggatatcg

SEQ ID NO: 52 - DNA, 27 bp, Artificial sequence
  primer
gctcagcacctcggcgtcgaaatctcc

SEQ ID NO: 53 - DNA, 17 bp, Artificial sequence
  primer
tctgccaatactccgcc

SEQ ID NO: 54 - DNA, 16 bp, Artificial sequence
  primer
ctttccggccggcatg

SEQ ID NO: 55 - DNA, 35 bp, Artificial sequence
  primer
ggtattgagggtcgcatgactgtcgccgatatcga

SEQ ID NO: 56 - DNA, 45 bp, Artificial sequence
  primer
agaggagagttagagcctacatcgaaaatagactgcttgtacacc

SEQ ID NO: 57 - DNA, 33 bp, Artificial sequence
  primer
ggtattgagggtcgcatgtcgcaacctgttgtg

SEQ ID NO: 58 - DNA, 39 bp, Artificial sequence
  primer
agaggagagttagagcctatatcttctcgagtttcttcc

SEQ ID NO: 59 - DNA, 33 bp, Artificial sequence
  primer
ggtattgagggtcgcatgggttccaacgccttc

SEQ ID NO: 60 - DNA, 37 bp, Artificial sequence
  primer
agaggagagttagagcctaaatggccctgccaaactg

SEQ ID NO: 61 - DNA, 37 bp, Artificial sequence
  primer
ggtattgagggtcgcatggctctccctgacgtcgaaa

SEQ ID NO: 62 - DNA, 35 bp, Artificial sequence
  primer
agaggagagttagagcctactcaaagatgctctcc

SEQ ID NO: 63 - DNA, 34 bp, Artificial sequence
  primer
ggtattgagggtcgcatgacagttccataccaag

SEQ ID NO: 64 - DNA, 42 bp, Artificial sequence
  primer
agaggagagttagagcctaatttacttctaatttagatgttc

SEQ ID NO: 65 - DNA, 33 bp, Artificial sequence
  primer
ggtattgagggtcgcatgtcggcagaaaagaag

SEQ ID NO: 66 - DNA, 39 bp, Artificial sequence
  primer
agaggagagttagagcccaaatccttgcacccttgcgcc

SEQ ID NO: 67 - DNA, 19 bp, Artificial sequence
  primer
cagaccaatggccagaaga

SEQ ID NO: 68 - DNA, 20 bp, Artificial sequence
  primer
agatgggcgatgtggtagtc

SEQ ID NO: 69 - DNA, 20 bp, Artificial sequence
  primer
gccgcttacagggaatgata

SEQ ID NO: 70 - DNA, 20 bp, Artificial sequence
  primer
atggctcaatctgcgagtct

SEQ ID NO: 71 - DNA, 20 bp, Artificial sequence
  primer
cgactcttgtgggtgctgta

SEQ ID NO: 72 - DNA, 20 bp, Artificial sequence
  primer
gtggaaaacacccattctgg

SEQ ID NO: 73 - DNA, 20 bp, Artificial sequence
  primer
ccccaatcgtcagatgaagt

SEQ ID NO: 74 - DNA, 20 bp, Artificial sequence
  primer
ctggcccacgattcactaat

SEQ ID NO: 75 - DNA, 20 bp, Artificial sequence
  primer
caaaagatcgccatccaact

SEQ ID NO: 76 - DNA, 20 bp, Artificial sequence
  primer
ctggtgacgagtccctcaat

SEQ ID NO: 77 - DNA, 22 bp, Artificial sequence
  primer
ccagcagatgttcgaccccaag

SEQ ID NO: 78 - DNA, 24 bp, Artificial sequence
  primer
cagtgaactccatctcgtccatac

SEQ ID NO: 79 - DNA, 18 bp, Artificial sequence
  primer
tccgtggcgtcaccttcc

SEQ ID NO: 80 - DNA, 21 bp, Artificial sequence
  primer
cagatgggcgatgtggtagtc

SEQ ID NO: 81 - DNA, 5387 bp, Artificial sequence
  plasmid pZVK2
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgacagctgtctcttatacacatctcaaccatcatcgatgaattttctcgggtgttctcgcatattggctcgaattcgagctcggtacccggggatcctctagaagtcctgaatagtagtttgtggattaacattgttccgatgtaggaatcatgatcccaaccagaagagctggacagcccctcttccagagcatttttggtgggatgttttggcttagtgcgatgcaactggacaaagtccttccgtttctactgcgtcttacatcatctggtatctacgcaagccgcccacttaccatatgaataagaggcactcaggttttccctcacccccccgaagcgatggtaagcgggtgccaaatgcatcgggagtttctctatcataataacctaggtattccgtaatctattaccagtctttccgaagagctggtagcaactgcacgagatttgtaggagcgagtacccggctggacgagcacgcagcacggctattggtcagcatggtagctaccgaggggaggcaggccgcccaaatatcgtgagtctcctgctttgcccggtgtatgaaaccggaaaagctgctatagagcttctgggcggcgcatgtcgggaaaccagcagcaagctgacccagaaagacccgtcctcaagccattaccgtactaatcaattatttgtgtagcaacactgggaagctgtagtgcataggctggagcagctatttggcctttagccccgtctgtccgcccggtgtgcggtttcgactggcgcgcaagctcaaggtgatcaggtcgttgcgtcagtcggagacaacaagccattgccttttctactgcccctcccccgctggtggcctttttctctcatcttctcctctcttcccatcatcagcatcattaatctactgtctctctttctttctatcattctataaagtaagaacatatccatcttccctcaatcccgtctacaatagtgtcctcttcactactctgtctctatctctcaaagcttgactgacatttaccccgctcagtaccagacgaatctacacagaattcgagctcactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgagaattccactagtgcagaaagctgttttccttgctctgtggtataagtctagtgccactattctatgatgagttgatgactctttcatgactggaaggcttacattctccaagatcatgtctcactcaaaacttatctcgggttcactttcgggttccatatatctcatcatttctgggtttagaaacatctctctcgtttttgcagctcttctacgtactcctagcggtttcactgaaatgaatacatttgggtaacctaattgccaattcatatcttcctgagggcagtaacacatcacgtacattctatcagctgtgatagagttacaaaactagcaatacttttatgcttcctcctttcttaccatttacacatccgctttctctctgctcttgatcttggcccctgattgtattgtcacctcaccaaattcaagtcatcacctcttctctagagtcgacttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctcgcatgcatcactagtgaattcgcggccgcctgcaggtcgacctgcaggcatgcaagcttgccaacgactacgcactagccaacaagagcttcagggttgagatgtgtataagagacagctgtcttaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 82 - DNA, 1326 bp, Fusarium graminearum
atgacagttcaatcacagcaacaatcccaggctattcccgtcctttcttcccagaatggcactgaaccccaagacgcaaacaaggaggttgttcagaatgtcgctgccaaaggagtgcaatacttcaaccctgagcaacttcctgcaccaggtctcggtataaacggtcccaataatactctaccaaaggtctttacacccatcaagattcgcggcatgaccatgcccaaccgtatctgggtcagccccatgtgccaatacagtgcccgtgacggctttcagcagccttggcactttgcccactacggcggactggcccaacgtggccctggcctcatcatgctagaagctaccgcagttcaagcacgtggccgtatcacacctgaagattctggcatctggctagactctcatgttgagggactgcgaaagcacgtcgagtttgcccatgccaacaactctcttatcggtatccagattggccatgctggtcgcaaggcctcctgcgttgctccttggttagacgccggacttgccgctgaaaaggccgctggtggatggcccgatgacgttgtcggacctagcaacgagccttttgctcctggctaccctaccccccgtgctattactcttgaagagattgaacagttgaaggaggactttgtttccggtgttcgtcgagcggttgaagcaggatttgacactatcgacttccatttcgctcacggttatcttgtttccagcttcctgtcccctgccaccaacaagcgtaccgacaagtacggaggtagcttcgagaacagagtgcgccttgctctcgagattgtcgaggctgcacgagctgttatgcctgaggacatgcccttgttcactcgcatcagtggaactgactggctggagaacaaccctgagtacgagggagagacctggactcttgagcagagcatcaagcttgcacaccagttagcagaccgtggtgtcgatgttttggatgtttccagtggtggcatccacaagatgcaaaaggtcgctgctggtcccggttaccaggcacctcttgccaaggcgatcaagaagtcagttggagacaagatgttgatcagcactgttggtagcatcaagataggtacccttgcggaggagatcatcgctggaggagaggacgatacccccttggatcttgtggcttcaggccgtctgttccagaagaacactggacttgtttggtcatgggctgacgatctgaacacttctatccagatcgctcatcagatcgcatggggtttcggtggcagagctaagaagaacgctcccaagcttgtctta

SEQ ID NO: 83 - protein, 442 aa, Fusarium graminearum
MTVQSQQQSQAIPVLSSQNGTEPQDANKEVVQNVAAKGVQYFNPEQLPAPGLGINGPNNTLPKVFTPIKIRGMTMPNRIWVSPMCQYSARDGFQQPWHFAHYGGLAQRGPGLIMLEATAVQARGRITPEDSGIWLDSHVEGLRKHVEFAHANNSLIGIQIGHAGRKASCVAPWLDAGLAAEKAAGGWPDDVVGPSNEPFAPGYPTPRAITLEEIEQLKEDFVSGVRRAVEAGFDTIDFHFAHGYLVSSFLSPATNKRTDKYGGSFENRVRLALEIVEAARAVMPEDMPLFTRISGTDWLENNPEYEGETWTLEQSIKLAHQLADRGVDVLDVSSGGIHKMQKVAAGPGYQAPLAKAIKKSVGDKMLISTVGSIKIGTLAEEIIAGGEDDTPLDLVASGRLFQKNTGLVWSWADDLNTSIQIAHQIAWGFGGRAKKNAPKLVL

SEQ ID NO: 84 - DNA, 1350 bp, Ustilago maydis
atggacacgtctcgattcgtgtctggtctcacaccgcctctcgtcgactcgatcgatgcactcaagatcagcaactttgtccccactcgaagtggccaccctcctcctggctcggtcccggaatccatcctgccagagggtgtcaaaaaaccggctttgttccaaacgttgacattgccctttgctgcaccggaacaggcgggtaagatgaccttcaagaaccgcatcattgtctctcccatgtgccagtactctgcgaacaatggtcttcctactccgtaccacattgcgcatttgggatcgtttgccctgcacggtgtgggaaacgtcatggtcgaagcatctggtgttgagccagaggggaggatcacgcctcaggacctgggtatttggtcggaacagcatcgggatgcacacaaggcgctggtgtcggtgctcaagtccttcacggatggtctgggtgtagggctgcaactggcgcatgcgggaaggaaggcctcggactggtcacctttctaccgcggagaaaagaagcaaaagtttgtgacgcaggaggaaggtggctggccggatcgtgtcgtcgctccttcggccatcgcatatgcgcaaggtcacgttacccctcgagctctcacgaccgaggacatcaacaagttgcaagacaaattcgttcagtcggcacgatgggcgtttgaagctgggtatgactacgtcgaacttcacagcgctcacggatacctgatgcactcgttcctcagcccgttgaccaatcagcgtaccgacgagtacggcggtagcctggagaaccgcgctcgatttctgctcaacgttgcccgtcgaatccgccaagaattccccaacaagggtctctgggtgcgcgtcagctccaccgactgggccgaccaagcgcaccaagccgactcttggaccgttgaccagacggttgaactcgccaagatgctccaagaggctcgagtcgacctgctagacgtcagctccggcggcctggttccattccaaaaaatcaccgtgggagccggataccagctattcggagcaaaagccgttcgcgatgctctggccaaaatcgaacccgacgcgtccaaacgcatgctcgtcggggccgtgggaatgatggaaggttcctacgattcgcccaacggccaagaccgcagccagattggcaagttggccgagcagtcgattcagagcggagagtgtgatgcggtactgttggcacgtggattgatgtcctacccaagctggaccgaggatgctagtgtagcgctgatgggtaccagggcagctggcaacccgcagtaccatcgcgttcacgtggctaagaagtga

SEQ ID NO: 85 - protein, 449 aa, Ustilago maydis
MDTSRFVSGLTPPLVDSIDALKISNFVPTRSGHPPPGSVPESILPEGVKKPALFQTLTLPFAAPEQAGKMTFKNRIIVSPMCQYSANNGLPTPYHIAHLGSFALHGVGNVMVEASGVEPEGRITPQDLGIWSEQHRDAHKALVSVLKSFTDGLGVGLQLAHAGRKASDWSPFYRGEKKQKFVTQEEGGWPDRVVAPSAIAYAQGHVTPRALTTEDINKLQDKFVQSARWAFEAGYDYVELHSAHGYLMHSFLSPLTNQRTDEYGGSLENRARFLLNVARRIRQEFPNKGLWVRVSSTDWADQAHQADSWTVDQTVELAKMLQEARVDLLDVSSGGLVPFQKITVGAGYQLFGAKAVRDALAKIEPDASKRMLVGAVGMMEGSYDSPNGQDRSQIGKLAEQSIQSGECDAVLLARGLMSYPSWTEDASVALMGTRAAGNPQYHRVHVAKK

SEQ ID NO: 86 - protein, 363 aa, Pseudomonas putida
MSALFEPYTLKDVTLRNRIAIPPMCQYMAEDGLINDWHQVHYASMARGGAGLLVVEATAVAPEGRITPGCAGIWSDAHAQAFVPVVQAIKAAGSVPGIQIAHAGRKASANRPWEGDDHIGADDARGWETIAPSAIAFGAHLPNVPRAMTLDDIARVKQDFVDAARRARDAGFEWIELHFAHGYLGQSFFSEHSNKRTDAYGGSFDNRSRFLLETLAAVREVWPENLPLTARFGVLEYDGRDEQTLEESIELARRFKAGGLDLLSVSVGFTIPETNIPWGPAFMGPIAERVRREAKLPVTSAWGFGTPQLAEAALQANQLDLVSVGRAHLADPHWAYFAAKELGVEKASWTLPAPYAHWLERYR

SEQ ID NO: 87 - protein, 359 aa, Streptomyces coelicolor
MSALFEPFRLRDTTIPNRIWMPPMCQYSAAPEGPSAGVPGDWHFAHYGARAVGGTGLIVVEATGVSPEGRISPQDLGLWNDTQVEAFRRITGFLRSQGTVPAVQLAHAGRKASTAQPWRGGAPVGADAYGWQPLAPSALAFDERHPVPTELTVPQIQEAVGRFADAARRALAAGFEIAEIHGAHGYLIHEFLSPHSNQRTDAYGGSYANRTRFALEVVDAVREVWPDDKPLFFRVSATDWLEEGGWTPDDTVRFARDLEAHGIDLLDVSTGGNVPRVRIPTGPGYQVPFAARVKAGSTLPVAAVGLITEPGQAEKILANGEADAVLLGRELLRNPSWAQHAARELGVDARMPDQYGWGM

SEQ ID NO: 88 - protein, 370 aa, Deinococcus radiodurans
MTVSSAAAPQPASPAAPLLFTPLKLRSLELPNRVVVSPMCTYSATDGVANEFHLVHLGQYALGGAGLILAEATAVSPEGRITPEDLGLWDDRQIVPLGHITDFVHQHGGHIGVQLAHAGRKASTYAPWRGKGAVPAELGGWQVIGPDENSFHDLFPTPAMMGADELRGVVDAFSAAARRAQVAGFDAVEVHAAHGYLLHQFLSPLANTRTDDYGGSFENRTRLLLEVVRAVRHVWPAHLPLFVRLSATDWAEGGWDLEQTVQLSKLLKYEGVDVLDISSGGLTAAQQIEVGPGYQVPFAAAVSRAETEISVMAVGLIETGAQAEAILQAGDADLIALGRPFLRDPHWAQRAARELGLRPVSIDQYARAGW

SEQ ID NO: 89 - protein, 773 aa, Azoarcus evansii
MRIVCIGGGPAGLYFAILMKKLNPAHEIRVIERNRPYDTFGWGVVFSDATMDNMREWDSETADAIQVAFNHWDDIELHFKGRTIRSGGHGFVGIGRKMMLNILQARCEELGVELVFDREVESDAEFPDADLVIASDGINSRIRNKYAEVFKPDIVTRPNRYIWLGTTKLFDAFTFFFEKTEHGWFQAHIYKFDDKTTTFIVECPEHVWKAHGLDTADQEQSIAFCEQLFGKHLDGHRLMTNSRHLRGSAWLNFQRVKCEQWHHYNGKSHVVLMGDAVHTAHFAIGSGTKLALEDAIELTRLFRDEGDTREHIPAVLERYQAARNIDVLRLQNAAWNAMEWFEVCGARYCDTLEPEQFMYSMLTRSQRISHENLRLRDAGWLEGYERWLARKAGMTVRDDETPPPPMFTPFKLRGLTLANRIVMSPMAMYSAEDGAPTDFHLVHFGSRALGGAGLLYTEMTCVSPDARITPGCAGMYKPEHVNAWKRIVDFVHGNSDAKIGMQLGHAGRKGATKLAWEGIDEPLEAGAWELISASPLPYLPHSQVPRAMTRDDMERVRNDFVRATRMAAEAGFDILELHCAHGYLLSSFLSPLTNRRTDEFGGDLENRARFPLEVFKAMRAMWPTNRPMSVRLSCHDWFPGGNTADDAVAIARLFKEAGADIIDCSSGQVWKGDQPVYGRMYQTPFADRIRNEVGIPTLAVGAISEADHANSIIAAGRADLCAIARPHLADPAWTLHEAAKIGFGEVAWPKQYRSARGQYETNLQRAAAAVAGK

SEQ ID NO: 90 - protein, 376 aa, Aspergillus fumigatus
MREEPSSAQLFKPLKVGRCHLQHRMIMAPTTRFRADGQGVPLPFVQEYYGQRASVPGTLLITEATDITPKAMGYKHVPGIWSEPQREAWREIVSRVHSKKCFIFCQLWATGRAADPDVLADMKDLISSSAVPVEEKGPLPRALTEDEIQQCIADFAQAARNAINAGFDGVEIHGANGYLIDQFTQKSCNHRQDRWGGSIENRARFAVEVTRAVIEAVGADRVGVKLSPYSQYLGMGTMDELVPQFEYLIAQMRRLDVAYLHLANSRWLDEEKPHPDPNHEVFVRVWGQSSPILLAGGYDAASAEKVTEQMAAATYTNVAIAFGRYFISTPDLPFRVMAGIQLQKYDRASFYSTLSREGYLDYPFSAEYMALHNFPV

SEQ ID NO: 91 - protein, 409 aa, Aspergillus fumigatus
MTIRKLDGEESMLFQPLEIANGRIRLSHRVVHAPMTRNRGVPLNPTSTPEQPNRIWYPGDLMVQYYRQRATPGGLIISEGVPPSLESNGMPGVPGLWTPEQAAGWKRVVDAVHEQGGYIYCQLWHAGRATIPQMTGSPAVSASATVWDSPTECYSHPPVGSTEPVRYADHPPIELTIPHLKQTIRDYCNAAKTAMEIGFDGVELHAGNGYLPEQFLSSNVNKRTDEYGGSPEKRCRFVLELMDELAATVGEDNLAIRLSPFGLFNQARGEQRVETWTFLCESLKKAHPNLSYVSFIEPRYEQIFSYEEKDNFLRSWGLSDVDLSSFRKIFGTTPFFSAGGWDQSNSWGVLEEGRYDALLYGRYFTSNPDLVERLRKGIPFTPYDRSRFYGPFEDNAKCYVDYPPATASS

SEQ ID NO: 92 - protein, 406 aa, Candida albicans
MTVESTNSFVVPAGTKQIEIAPLGSTKLFQPIKVGKNILPHRVAHAPTTRFRAAKNHTPSDLQLEYYKTHSQYPGTLIITEATFTSEQGGMDLHVPGIYNDAQTKAWKKINDEIHANGSFSSMQLWYLGRVANPKDLKDAGLPLIGPSAVYWDEESEKLAKSVGNELRELTEKEIDHIVEVEYPNAAKRAIEAGFDYIEVHSAPGYFLDQFLNPASNKRTDKYGGSIENRARLLLRIIDKLIGIVGAEKLAVRLAPWSSFLGMEIEGEEIHSYILQQLQQRADNGQQLAYVSLIEPRVIGIFDASLEDQKGRSNEFAYKYWKGNFVRAGNYTYDAPEFKTLLHDLDNDRTIVGFARFFTSNPDLVEKLKLGKPLNHYDREEFYKYYNYGYNSYDESEKQVIGKPLV

SEQ ID NO: 93 - protein, 406 aa, Candida albicans
MTIESTNSFVVPSDTKLIDVTPLGSTKLFQPIKVGNNVLPQRIAYVPTTRFRASKDHIPSDLQLNYYNARSQYPGTLIITEATFASERGGIDLHVPGIYNDAQAKSWKKINEAIHGNGSFSSVQLWYLGRVANAKDLKDSGLPLIAPSAVYWDENSEKLAKEAGNELRALTEEEIDHIVEVEYPNAAKHALEAGFDYVEIHGAHGYLLDQFLNLASNKRTDKYGCGSIENRARLLLRVVDKLIEVVGANRLALRLSPWASFQGMEIEGEEIHSYILQQLQQRADNGQQLAYISLVEPRVTGIYDVSLKDQQGRSNEFAYKIWKGNFIRAGNYTYDAPEFKTLINDLKNDRSIIGFSRFFTSNPDLVEKLKLGKPLNYYNREEFYKYYNYGYNSYDESEKQVIGKPL

SEQ ID NO: 94 - protein, 379 aa, Neurospora crassa
MAATAAESRLFQPLKLTPKITLGHRLAMAPLTRFRSDDEHVPIVPLMTTYYSQRASVPGTLLVTEATFISPAAGGYDNVPGIYNAAQIAAWKKITDAVHAKGSFIFCQLWSLGRAANPEVLAKEGGLKLKSSSAVPMEEGAPVPEEMTVAEIKERVAEYAAAAKNAVEAGFDGVEIHGANGYLIDQFLQDTCNQRTDEYGGSIENRSRFAHEVVKAVVEAVGAEKTGIRLSPYSTFQGMKMKKDLIPQFEDVIRKINGFGLAYLHLTQSRVAGNMDVQPEEDEENLAFAAKLWDGPLLIAGGLTPETAKHLVDREFPEKDVVATFGRHFISTPDLPFRIKEGIELNPYDRDTFYKAKSPDGYIDQPFSKEFEKVYGAQA

SEQ ID NO: 95 - protein, 400 aa, Saccharomyces cerevisiae
MSFVKDFKPQALGDTNLFKPIKIGNNELLHRAVIPPLTRMRALHPGNIPNRDWAVEYYTQRAQRPGTMIITEGAFISPQAGGYDNAPGVWSEEQMVEWTKIFNAIHEKKSFVWVQLWVLGWAAFPDNLARDGLRYDSASDNVFMDAEQEAKAKKANNPQHSLTKDEIKQYIKEYVQAAKNSIAAGADGVEIHSANGYLLNQFLDPHSNTRTDEYGGSIENRARFTLEVVDALVEAIGHEKVGLRLSPYGVFNSMSGGAETGIVAQYAYVAGELEKRAKAGKRLAFVHLVEPRVTNPFLTEGEGEYEGGSNDFVYSIWKGPVIRAGNFALHPEVVREEVKDKRTLIGYGRFFISNPDLVDRLEKGLPLNKYDRDTFYQMSAHGYIDYPTYEEALKLGWDKK

SEQ ID NO: 96 - protein, 400 aa, Saccharomyces cerevisiae
MPFVKDFKPQALGDTNLFKPIKIGNNELLHRAVIPPLTRMRAQHPGNIPNRDWAVEYYAQRAQRPGTLIITEGTFPSPQSGGYDNAPGIWSEEQIKEWTKIFKAIHENKSFAWVQLWVLGWAAFPDTLARDGLRYDSASDNVYMNAEQEEKAKKANNPQHSITKDEIKQYVKEYVQAAKNSIAAGADGVEIHSANGYLLNQFLDPHSNNRTDEYGGSIENRARFTLEVVDAVVDAIGPEKVGLRLSPYGVFNSMSGGAETGIVAQYAYVLGELERRAKAGKRLAFVHLVEPRVTNPFLTEGEGEYNGGSNKFAYSIWKGPIIRAGNFALHPEVVREEVKDPRTLIGYGRFFISNPDLVDRLEKGLPLNKYDRDTFYKMSAEGYIDYPTYEEALKLGWDKN

SEQ ID NO: 97 - protein, 400 aa, Saccharomyces cerevisiae
MPFVKGFEPISLRDTNLFEPIKIGNTQLAHRAVMPPLTRMRATHPGNIPNKEWAAVYYGQRAQRPGTMIITEGTFISPQAGGYDNAPGIWSDEQVAEWKNIFLAIHDCQSFAWVQLWSLGWASFPDVLARDGLRYDCASDRVYMNATLQEKAKDANNLEHSLTKDDIKQYIKDYIHAAKNSIAAGADGVEIHSANGYLLNQFLDPHSNKRTDEYGGTIENRARFTLEVVDALIETIGPERVGLRLSPYGTFNSMSGGAEPGIIAQYSYVLGELEKRAKAGKRLAFVHLVEPRVTDPSLVEGEGEYSEGTNDFAYSIWKGPIIRAGNYALHPEVVREQVKDPRTLIGYGRFFISNPDLVYRLEEGLPLNKYDRSTFYTMSAEGYTDYPTYEEAVDLGWNKN

SEQ ID NO: 98 - DNA, 26 bp, Artificial sequence
  primer
gctagcatgactgtcgccgatatcga

SEQ ID NO: 99 - DNA, 28 bp, Artificial sequence
  primer
gctagcctatacatcgaaaatagactgc

SEQ ID NO: 100 - DNA, 27 bp, Artificial sequence
  primer
actagtccaggggactgtcgtggtcaa

SEQ ID NO: 101 - DNA, 26 bp, Artificial sequence
  primer
caattgcccaggcctaatgcatgctg

(19) United States
(12) Patent Application Publication
(10) Pub. No.: US 2008/0206220 A1
(43) Pub. Date: Aug. 28, 2008

(54) 2031 Oxidoreductase

(75) Inventors: Sandra Elizabeth Lavens, Eccles (GB); Jason David Oliver, Eccles (GB); John Leslie Thain, Eccles (GB); Daniel Scott Tuckwell, Eccles (GB); Michael John Bromley, Eccles (GB)

Correspondence Address: FULBRIGHT & JAWORSKI, LLP, 1301 MCKINNEY, SUITE 5100, HOUSTON, TX, 77010-3095, US

(73) Assignee: F2G LTD, Greater Manchester (GB)

(21) Appl. No.: 10/590,171

(22) PCT Filed: Feb. 18, 2005
(86) PCT No.: PCT/GB2005/000623

§ 371 Date: Apr. 1, 2008

(30) Foreign Application Priority Data
Feb. 19, 2004 (GB) 0403746.1
Oct. 29, 2004 (GB) 0424080.0

(51) Int. Cl. A61K 38/44; C12Q 1/68; C12Q 1/26; C12N 9/02; C07H 21/00; A61K 31/7088; C12N 15/00; C12N 5/00; C07K 16/00; C12N 1/15

(52) U.S. Cl. 424/94.4; 435/6; 435/7.1; 435/189; 536/23.1; 435/320.1; 435/325; 530/387.9; 435/254.1; 514/44

(57) ABSTRACT

Method of identifying an anti-fungal agent which targets an essential protein or gene of a fungus comprising contacting a candidate substance with
(i) a NADH:flavin oxidoreductase protein which comprises the sequence shown by SEQ ID NO:3,
(ii) a NADH:flavin oxidoreductase protein which is a homologue of (i) and which comprises the sequence shown by SEQ ID NO: 8, 12, 14, 19, 24, 42, 44, 83 or 85,
(iii) a protein which has 50% identity with (i) or (ii),
(iv) a protein comprising a fragment of (i), (ii) or (iii) which fragment has a length of at least 50 amino acids,
(v) a polynucleotide that comprises sequence which encodes (i), (ii), (iii) or (iv),
(vi) a polynucleotide comprising sequence which has at least 70% identity with the coding sequence of (v),
and determining whether the candidate substance binds or modulates (i), (ii), (iii), (iv), (v) or (vi), wherein binding or modulation of (i), (ii), (iii), (iv), (v) or (vi) indicates that the candidate substance is an anti-fungal agent.

### CROSS-REFERENCE TO RELATED APPLICATIONS

This application is the National Phase Application of International Application No. PCT/GB2005/000623 filed Feb. 18, 2005, which claims priority to Great Britain Application Nos. 0403746.1 filed Feb. 19, 2004 and 0424080.0 filed Oct. 29, 2004.

### TECHNICAL FIELD

The present invention relates to a method of screening for an anti-fungal agent, to fungal 2031 oxidoreductase (2031 OR) enzymes and to diagnosis and therapy of fungal infections.

### BACKGROUND OF THE INVENTION

Oxidoreductases are a major class of enzymes (EC 1) that catalyse oxidation-reduction (redox) reactions. Redox reactions involve the transfer of reducing equivalents, in the form of electrons or hydrogen atoms, between molecules, i.e., from an electron donor (or reductant) to an electron acceptor (or oxidant). There are many different types of oxidoreductase important for many cellular processes from respiration to protein folding.

The NADH:flavin oxidoreductase/NADH oxidase family of enzymes (InterPro reference IPR001155) contains approximately 263 members mostly of bacterial or yeast origin but with some plant and nematode members. Members of this family use flavin mononucleotide (FMN) or flavin adenine dinucleotide (FAD) as a tightly bound prosthetic group. The flavin prosthetic group can exist in an oxidised (FMN or FAD) or a reduced form (FMNH₂ or FADH₂). These oxidoreductases use the reduced form of nicotinamide adenine dinucleotide (NADH) or nicotinamide adenine dinucleotide phosphate (NADPH) as the reductant. A variety of substrates can act as oxidants in the redox reaction.

Old Yellow Enzyme (OYE) is the oldest known member of this family of oxidoreductases (reviewed in Williams and Bruce, 2002, Microbiology 148, 1607-1614). OYE1 (EC 1.6.99.1) was isolated from brewer's bottom yeast by Warburg & Christian (1932, Naturwissenschaften 20, 688) and was the first enzyme for which a cofactor was shown to be required (Theorell, 1935, Biochem. Z. 275, 344-346). This yellow cofactor was found to be riboflavin 5′-phosphate (also known as flavin mononucleotide, FMN). There are 2 OYEs known in Saccharomyces cerevisiae (OYE2 & OYE3) and 2 in Schizosaccharomyces pombe. A great deal is known about the biochemical mechanism and structure of the enzyme, however, the precise physiological role of the enzyme remains to be elucidated.

OYE has NADPH dehydrogenase activity (see reaction 1 below). The reduced enzyme catalyses the reduction of α/β-unsaturated carbonyl compounds including cyclohexenone (see reaction 2), duroquinone, menadione and N-ethylmaleimide.

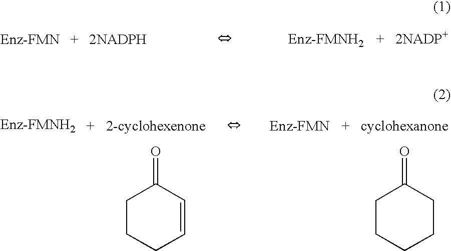

It has been speculated that OYE may be involved in sterol metabolism (Stott et al, 1993, J. Biol. Chem. 268: 6097-6106) or may be part of the antioxidant defense machinery involved in detoxification of, for example, lipid peroxidation breakdown products (Kohli & Massey, 1998, J. Biol. Chem. 273, 32763-32770). Neither OYE2 nor OYE3 are essential for S. cerevisiae.

Bacterial members of the NADH:flavin oxidoreductase family include Escherichia coli N-ethylmaleimide reductase, Pseudomonas putida M10 morphinone reductase, Enterobacter cloacae PB2 penterythritol tetranitrate reductase and Azoarcus evansii 2-aminobenzoyl-CoA monooxygenase/reductase (Schühle et al., 2001, J. Bacteriol. 183, 5268-5278).

### BRIEF SUMMARY OF THE INVENTION

The inventors have found a gene for an oxidoreductase of the NADH:flavin oxidoreductase type to be essential for the viability of fungal cells. This finding allows the identification of anti-fungal agents based on their ability to target the oxidoreductase.

The invention provides a new group of oxidoreductases which are herein referred to as 2031 oxidoreductases (2031 ORs) which can be used to screen for anti-fungal agents. In particular 2031 oxidoreductases from Aspergillus fumigatus, Aspergillus nidulans, Candida albicans, Colletotrichium trifolii, Fusarium graminearum (anamorph Gibberella zeae) Fusarium sporotrichoides, Magnaporthe grisea, Neurospora crassa, Schizosaccharomyces pombe and Ustilago maydis (see Table I) are provided. 2031 OR defines a novel set of oxidoreductases, related to but distinct from OYE and its close relatives, which are essential for the viability of fungal cells.

Accordingly the invention provides the following:
a method of identifying an anti-fungal agent which targets an essential protein or gene of a fungus comprising contacting a candidate substance with
(i) a NADH:flavin oxidoreductase protein which comprises the sequence shown by SEQ ID NO:3,
(ii) a NADH:flavin oxidoreductase protein which is a homologue of (i) and which comprises the sequence shown by SEQ ID NO: 8, 12, 14, 19, 24, 42, 44, 83 or 85,
(iii) a protein which has 50% identity with (i) or (ii),
(iv) a protein comprising a fragment of (i), (ii) or (iii) which fragment has a length of at least 50 amino acids,
(v) a polynucleotide that comprises sequence which encodes (i), (ii), (iii) or (iv),
(vi) a polynucleotide comprising sequence which has at least 70% identity with the coding sequence of (v),
and determining whether the candidate substance binds or modulates (i), (ii), (iii), (iv), (v) or (vi), wherein binding or modulation of (i), (ii), (iii), (iv), (v) or (vi) indicates that the candidate substance is an anti-fungal agent,
use of (i), (ii), (iii), (iv), (v) or (vi) as defined above to identify or obtain an anti-fungal agent,
use of an anti-fungal agent identified by the method of the invention in the manufacture of a medicament for prevention or treatment of fungal infection,
a method of detecting the presence of a fungus in a sample comprising detecting the presence in the said sample of a protein or polynucleotide of the invention,
an isolated protein or polynucleotide of the invention,
an organism which is transgenic for a polynucleotide of the invention,
an organism which has been genetically engineered to render a polynucleotide or protein of the invention non-functional or inhibited.
an antibody which is specific for a protein of the invention,
a method for preventing or treating a fungal infection comprising administering an anti-fungal agent identified by the screening method of the invention, and
a fungus which has been killed, or whose growth has been impaired, by inhibition of the expression or activity of a protein or polynucleotide of the invention.

### BRIEF DESCRIPTION OF THE DRAWINGS

Embodiments of the invention will now be described by way of example, with reference to the accompanying drawings in which:—

FIG. 1 illustrates a multiple sequence alignment of amino acid sequences corresponding to fungal and bacterial 2031 and OYE family oxidoreductases;

FIG. 2 illustrates a multiple sequence alignment of nucleic acid sequences corresponding to fungal 2031 and family oxidoreductases;

FIG. 3A illustrates the expression of recombinant 2031 OR; B shows purified recombinant 2031 OR.

FIG. 4. Phylogenetic tree showing relationships between A. fumigatus 2031 OR and similar proteins. This demonstrates a 2031 OR clade, which can be distinguished from the OYE proteins;

FIG. 5 illustrates reduction of a range of substrates by recombinant 2031 OR.

FIG. 6 illustrates the inhibition of 2031 OR by two compounds identified from a screen.

### DETAILED DESCRIPTION OF THE INVENTION

As mentioned above the invention relates to use of particular protein and polynucleotide sequences (termed “proteins of the invention” and “polynucleotides of the invention” herein) which are of, or derived from, fungal oxidoreductase proteins and polynucleotides (including homologues and/or fragments of the fungal oxidoreductase proteins and polynucleotides) to identify anti-fungal agents.

As used herein, the term “oxidoreductase” (“OR”) may be defined as an enzyme or which is capable of catalysing an oxidation or reduction reaction. The protein of the invention may have an oxidation or reduction activity, such any such activity mentioned herein. The ORs of the invention generally fall within classification EC1 of the enzyme commission.

An essential fungal gene may be defined as one which, when disrupted genetically (for example when not expressed) in a fungus, prevents survival or significantly retards growth of the cell on minimal or defined medium, or in guinea pigs, mice, rabbits or rats infected with the fungus. In one embodiment the protein of the invention is able to complement such an effect of the genetic disruption. Thus the protein may cause survival (viability) of a fungal cell which does not express its native 2031 oxidoreductase.

A protein or polynucleotide of the invention (or a fungal “2031 OR” gene, nucleic acid or protein) may be defined by similarity in sequence to a another member of the family. As mentioned above this similarity may be based on percentage identity (for example to the sequences shown in the sequence listing).

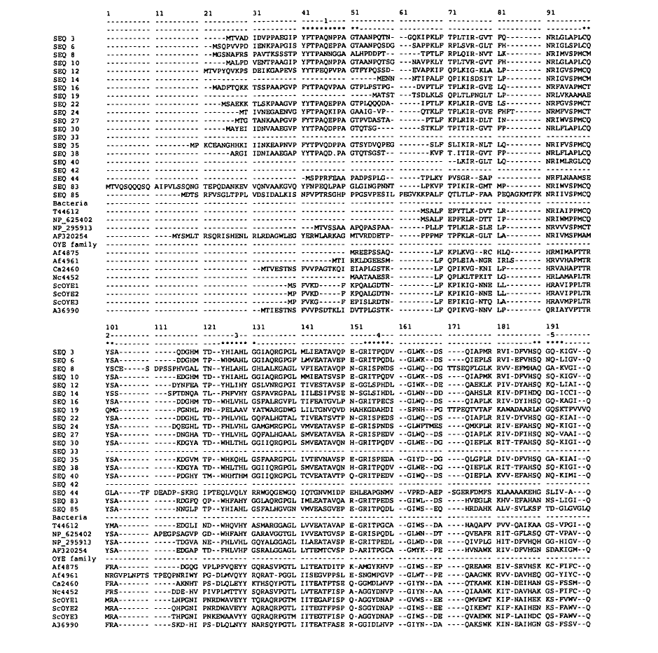

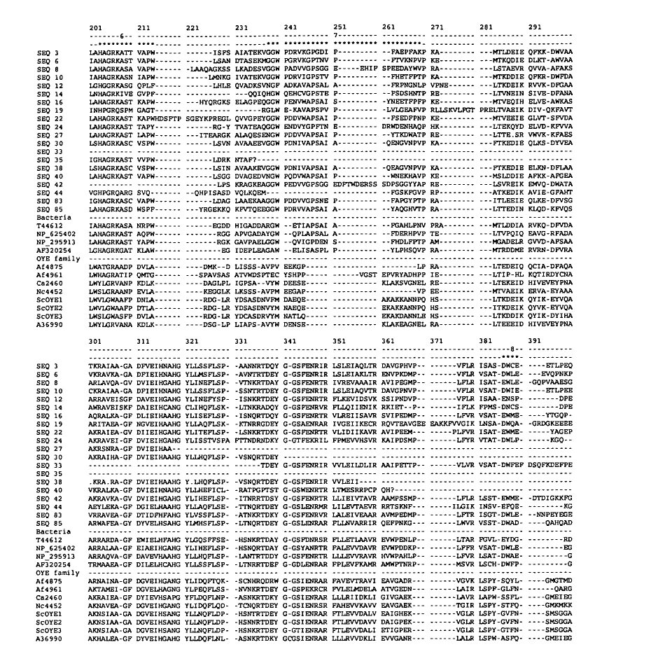

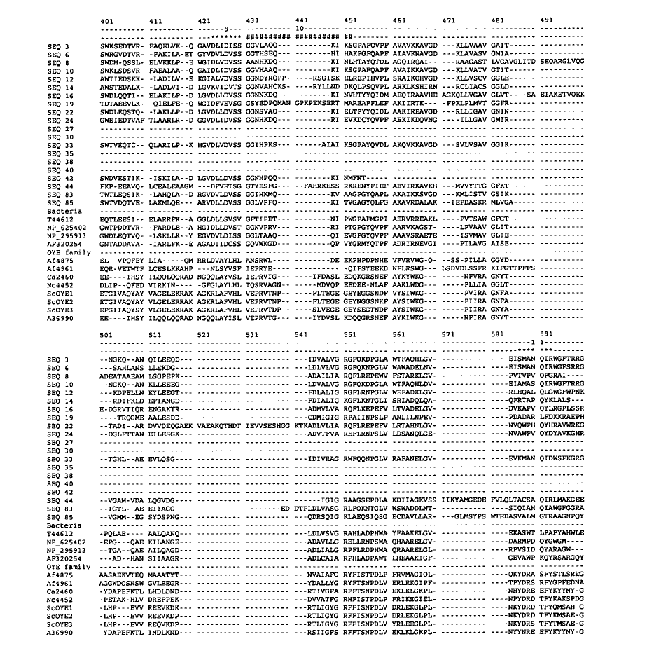

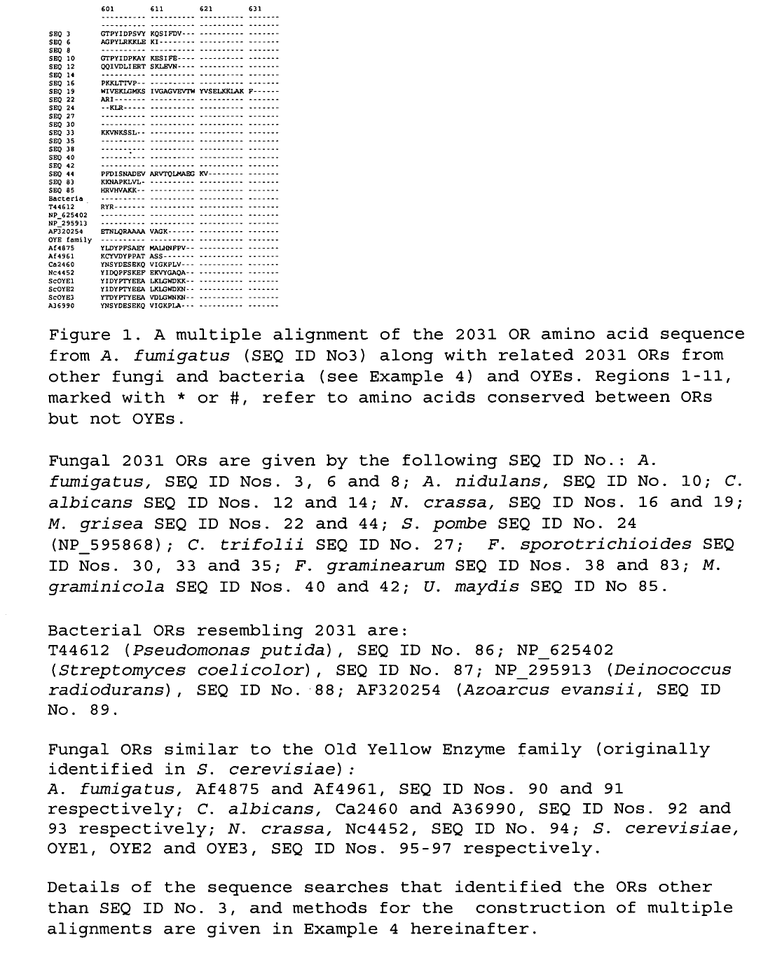

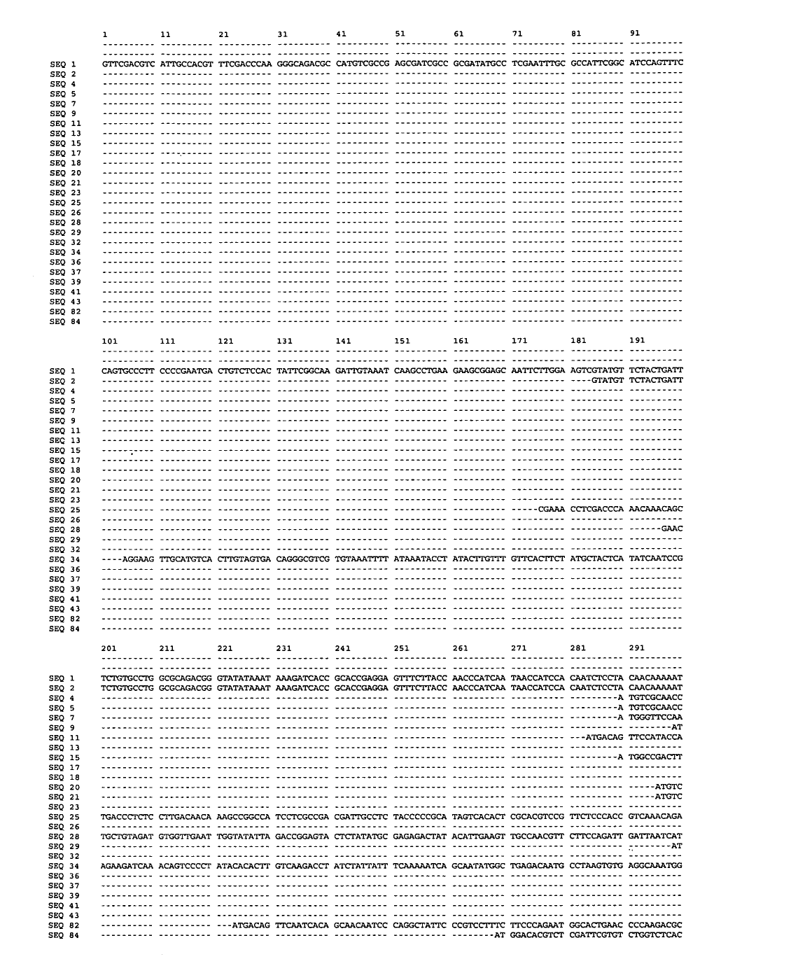

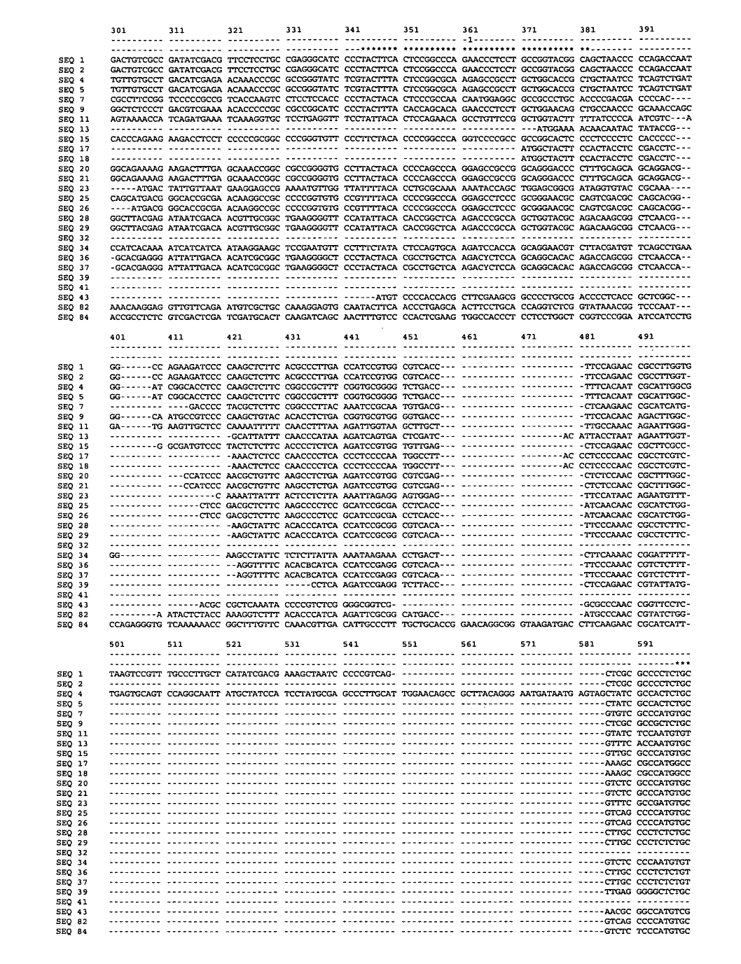

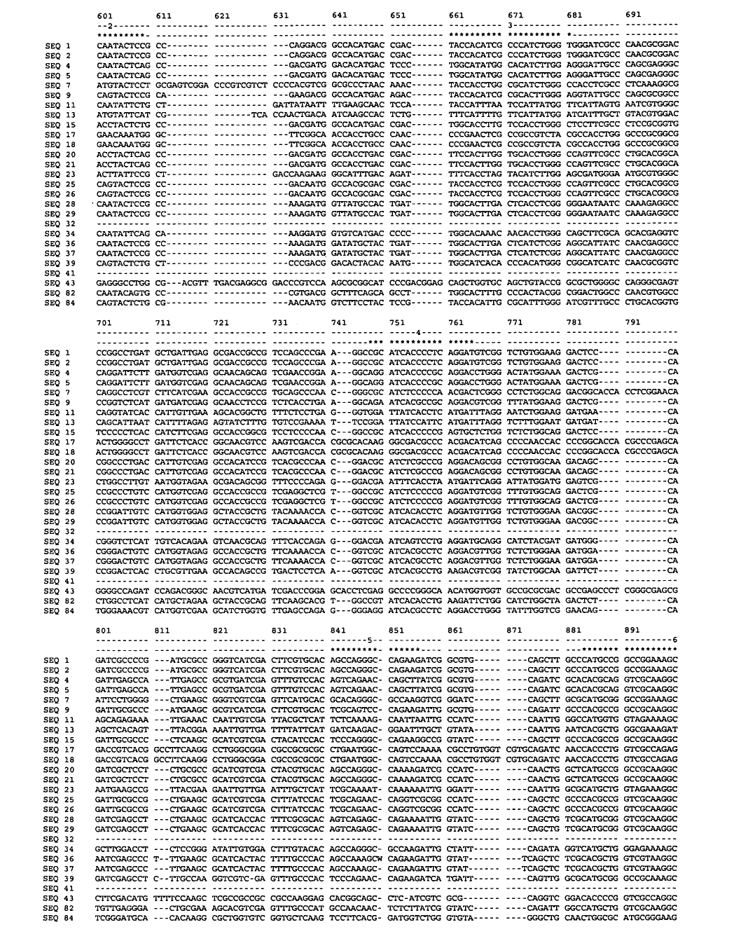

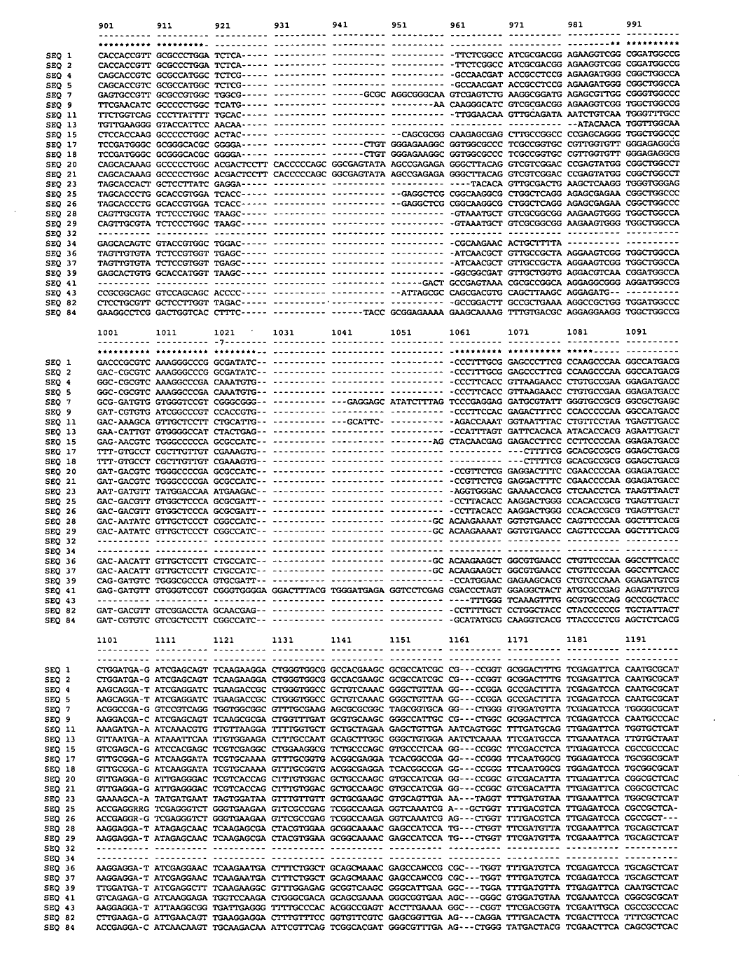

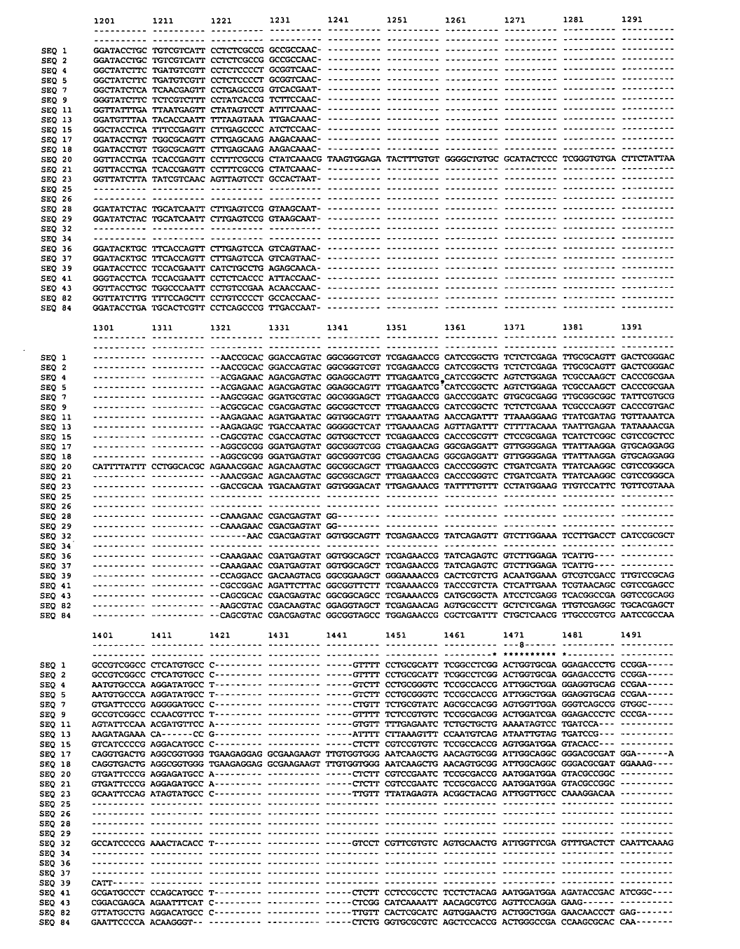

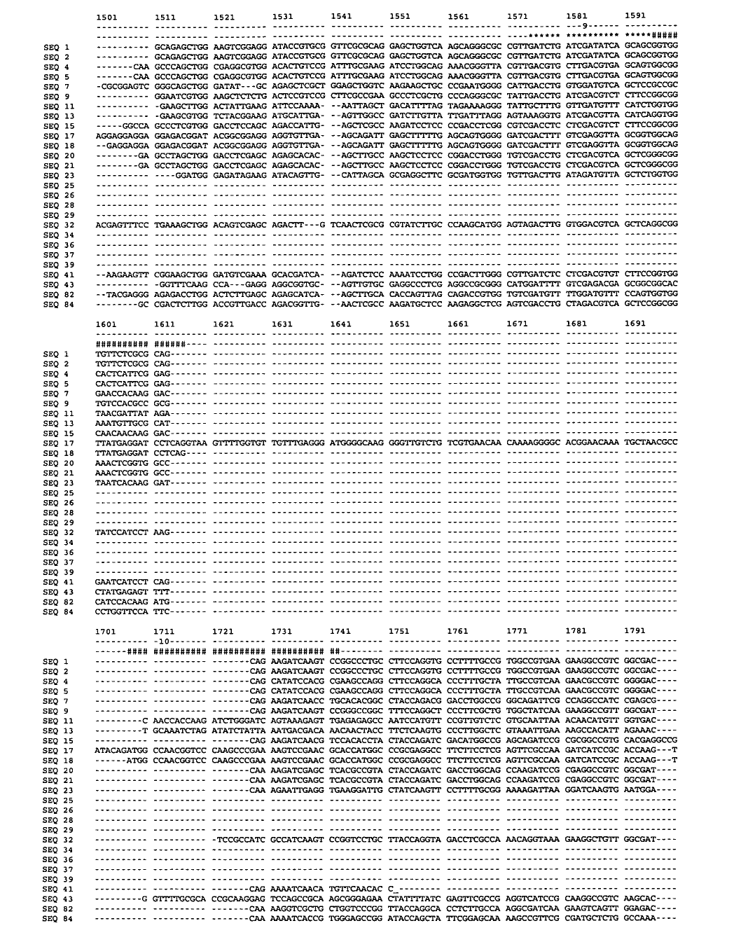

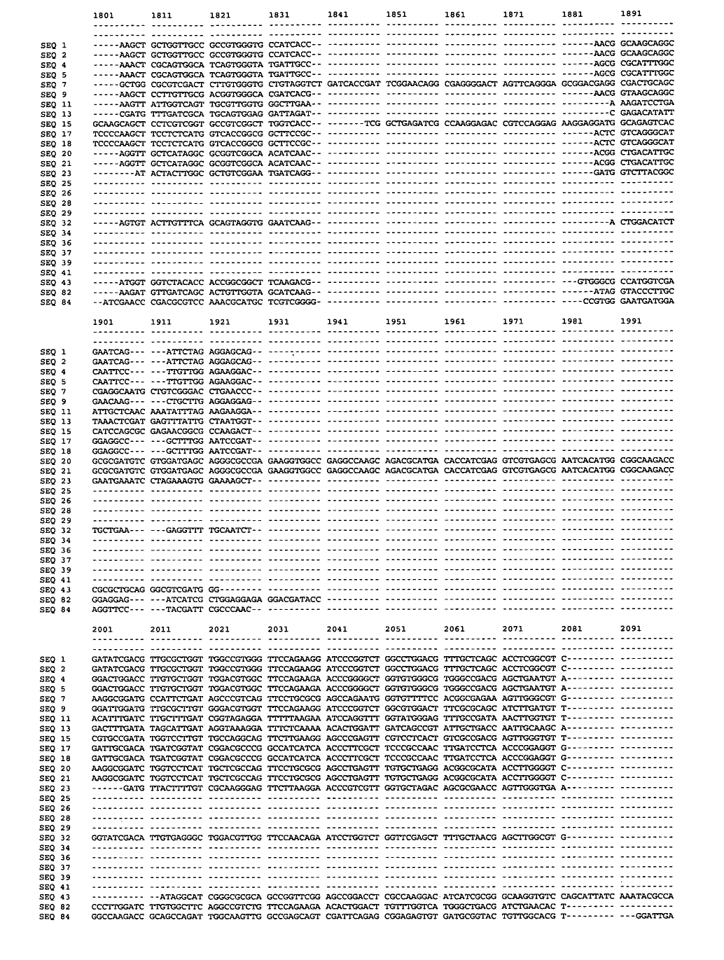

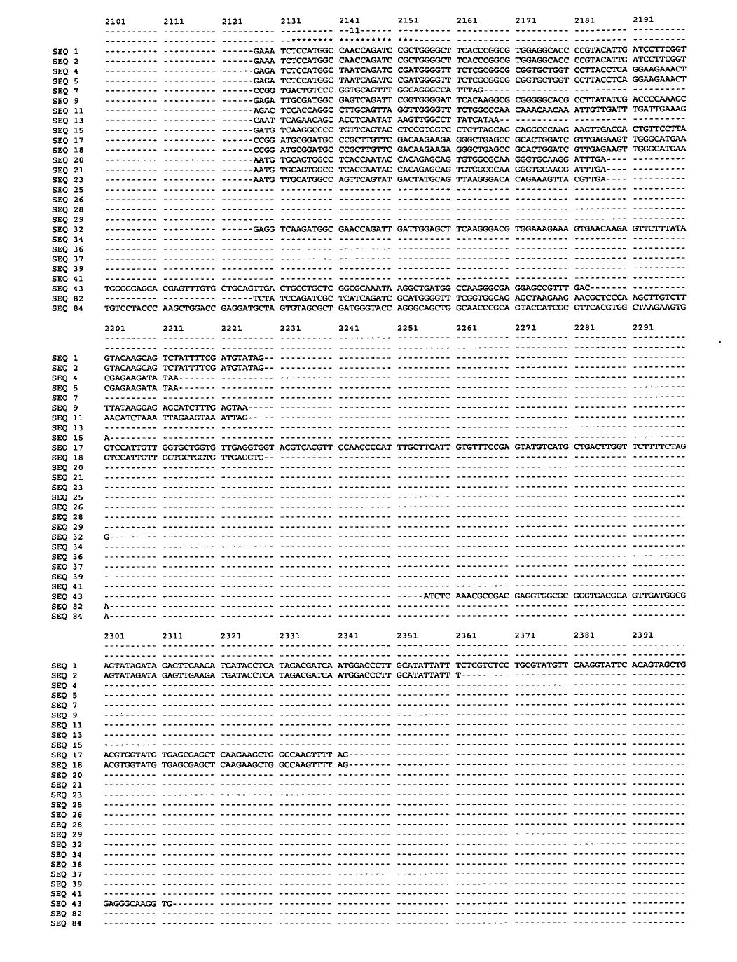

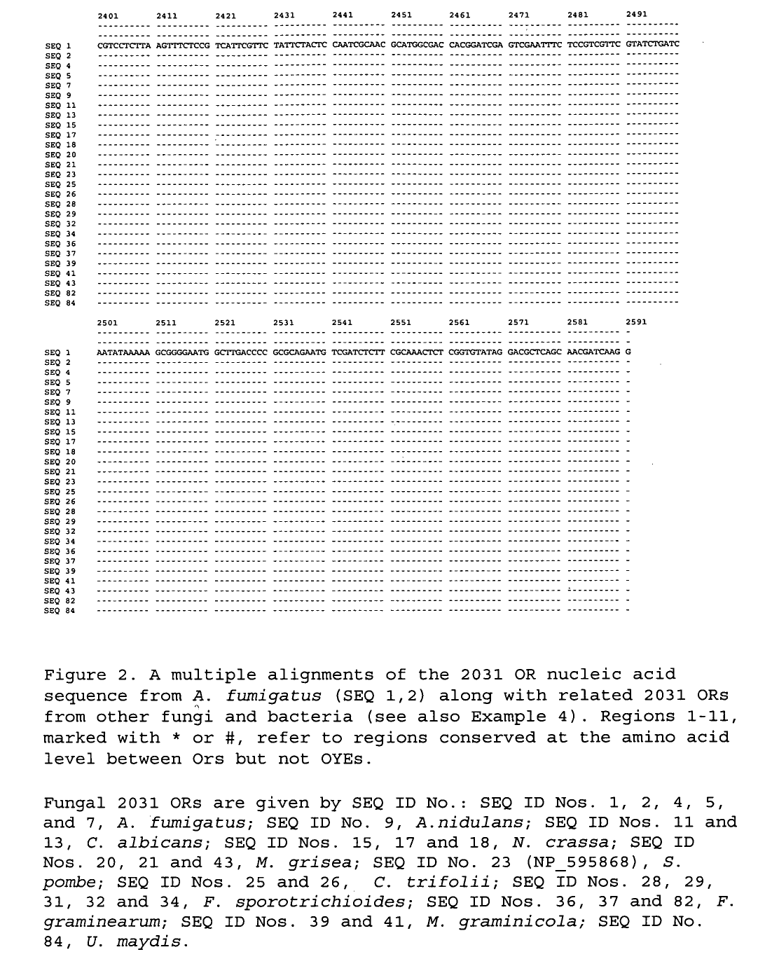

A protein or polynucleotide of the invention may comprise one or more of the motifs defined by regions 1-11 of FIGS. 1 and 2 (marked at the top of the Figures) of any of the sequences shown. Thus a protein of the invention may comprise one or more of motifs 1-11 as shown for SEQ ID NO:3 and a polynucleotide of the invention may comprise one or more of motifs 1-11 as shown for SEQ ID NO:1.

Typically the motif is present in substantially the same location as the equivalent location shown in FIG. 1 or 2. The equivalent location can be deduced, for example, using any suitable algorithm mentioned herein. In one embodiment the protein or polynucleotide also comprises sequence flanking the motif as shown in FIG. 1 or 2 such as sequences of length at least 10, 20 or 30 amino acids/nucleotides flanking the N terminal side and/or C terminal side, or 5′ and/or 3′ side, of the motif, or sequence which has percentage identity with the flanking sequence.

The protein of the invention typically comprises at least 2, 3, 5, 8 or 11 of the motifs shown in FIGS. 1 and 2. The protein preferably comprises at least motif no. 6 and/or motif no. 9.

The protein or polynucleotide of the invention may align with other 2031 OR polynucleotides or proteins (as shown in SEQ ID Nos. 1-44 and 82-85) showing a greater identity to these than to Old Yellow Enzyme family polynucleotides or proteins

The protein or polynucleotide of the invention typically clusters with other 2031 OR polynucleotides or proteins (as shown in SEQ ID Nos. 1-44 and 82-85) rather than Old Yellow Enzyme family polynucleotides or proteins after phylogenetic analysis, for example with a bootstrap value of greater than 60%.

In one embodiment the protein of the invention has a sequence which matches PFAM profile “oxidored FMN”, or INTERPRO profile IPR001155 (for example with an Evalue of e-50 or less) and is closer to a 2031 OR shown in any one of SEQ ID Nos. 1-44 and 82-85 than to Old Yellow Enzyme family proteins.

The protein or polynucleotide of the invention may be in isolated form (such as non-cellular form), for example when used in the method of the invention. Preferably, the isolated polynucleotide comprises a 2031 OR gene. Preferably, the isolated protein comprises a 2031 OR. The polynucleotide may comprise native, synthetic or recombinant polynucleotide, and the protein may comprise native, synthetic or recombinant protein. The polynucleotide or protein may comprise combinations of native, synthetic or recombinant polynucleotide or protein, respectively. The polynucleotides and proteins of the invention may have a sequence which is the same as, or different from, naturally occurring 2031 OR polynucleotides and proteins.

It is to be understood that the term “isolated from” may be read as “of” herein. Therefore references to polynucleotides and proteins being “isolated from” a particular organism include polynucleotides and proteins which were prepared by means other than obtaining them from the organism, such as synthetically or recombinantly.

Preferably, the polynucleotide or protein, is isolated from a fungus, more preferably a filamentous fungus, even more preferably an Ascomycete.

Preferably, the polynucleotide or protein, is isolated from an organism selected from Aspergillus; Blumeria; Candida, Colletotrichium; Cryptococcus; Encephalitozoon; Fusarium, Leptosphaeria; Magnaporthe; Mycosphaerella; Neurospora, Phytophthora; Plasmopara; Pneumocystis; Pyricularia; Pythium; Puccinia; Rhizoctonia; Schizosaccharomyces, Trichophyton; and Ustilago.

Preferably, the polynucleotide or protein, is isolated from an organism independently selected from a group of genera consisting of Aspergillus, Candida, Colletotrichium, Fusarium, Magnaporthe, Mycosphaerella, Neurospora, Schizosaccharomyces and Ustilago.

Preferably, the polynucleotide or protein, is isolated from an organism selected from the species Aspergillus flavus; Aspergillus fumigatus; Aspergillus nidulans; Aspergillus niger; Aspergillus parasiticus; Aspergillus terreus; Blumeria graminis; Candida albicans; Candida cruzei; Candida glabrata; Candida parapsilosis; Candida tropicalis; Colletotrichium trifolii; Cryptococcus neoformans; Encephalitozoon cuniculi; Fusarium graminarium; Fusarium solani, Fusarium sporotrichoides; Leptosphaeria nodorum; Magnaporthe grisea, Mycosphaerella graminicola; Neurospora crassa; Phytophthora capsici; Phytophthora infestans; Plasmopara viticola; Pneumocystis jiroveci; Puccinia coronata, Puccinia graminis; Pyricularia oryzae; Pythium ultimum; Rhizoctonia solani; Schizzosaccharomyces pombe; Trichophyton interdigitale; Trichophyton rubrum; and Ustilago maydis.

Preferably, the polynucleotide or protein, is isolated from an organism selected from Aspergillus fumigatus; Aspergillus nidulans, Candida albicans, Colletotrichium trifolii, Fusarium graminearum, Fusarium sporotrichoides, Magnaporthe grisea, minicola, Neurospora crassa, Schizosaccharomyces pombe and Ustilago maydis.

The polynucleotide, and preferably the protein, may be isolated from A. fumigatus AF293.

TABLE I
2031 OR sequences claimed and their relationship to sequences
given in the sequence listing.

| | | Coding | |
|---|---|---|---|
| | | sequence(cDNA/mRNA) | |
| | gDNA/EST¹ | w/o UTRs² | Protein |
| A. fumigatus | SEQ ID No. 1: | SEQ ID No. 2: 115-1384 | SEQ ID. No. 3 |
| Oxidoreductase 2031 | 299-469, 520-1618 | | |
| A. fumigatus | SEQ ID No. 4: | SEQ ID No. 5: 1-1266 | SEQ ID No. 6 |
| Oxidoreductase 4929 | 1-180, 267-1352 | | |
| A. fumigatus | SEQ ID No. 7: | SEQ ID No. 7: 1-1329 | SEQ ID No. 8 |
| Oxidoreductase 1495 | 1-1329 | | |
| A. nidulans 1_112 | SEQ ID No. 9: | SEQ ID No. 9: | SEQ ID No. 10 |
| | 1-1269 | 1-1269 | |
| C. albicans 2431 | SEQ ID No. 11: | SEQ ID No. 11 | SEQ ID No. 12 |
| | 1-1299 | 1-1299 | |
| C. albicans 2464 | SEQ ID No. 13: 1-1110 | SEQ ID No. 13: 1-1110 | SEQ ID No. 14 |
| N. crassa NCU07452.1 | SEQ ID No. 15: 1-1305 | SEQ ID No. 15: 1-1305 | SEQ ID No. 16 |
| N. crassa Oxidoreductase | SEQ ID No. 17: 1-924, 1015-1362, | SEQ ID No. 18: 1-1314 | SEQ ID No. 19 |
| NCU08900 | 1435-1476 | | |
| M. grisea MG04569.3 | SEQ ID No. 20: 1-726, 810-1412 | SEQ ID No. 21: 1-1329 | SEQ ID No. 22 |
| (pred gene) | | | |
| S. pombe T39956 | SEQ ID No. 23: 1-1188 | SEQ ID No. 23: 1-1188 | SEQ ID No. 24 |
| C. trifolii (EST assembly) | SEQ ID No. 25: 130-777 | SEQ ID No. 26: 1-645⁽³⁾ | SEQ ID No. 27 |
| F. sporotrichoides | SEQ ID No. 28: 103-803 | SEQ ID No. 29: 1-701 | SEQ ID No. 30 |
| FsCon[0063] (ESTs) | | | |
| F. sporotrichoides | SEQ ID No. 31: 76-631 (rev | SEQ ID No. 32: 1-556 | SEQ ID No. 33 |
| FsCon[0237] (ESTs) | comp) | | |
| F. sporotrichoides | SEQ ID No. 34: 174-657 | SEQ ID No. 34: 174-657 | SEQ ID No. 35 |
| FsCon[0458] (ESTs) | | | |
| F. graminearum | SEQ ID No. 36: 1-744 | SEQ ID No. 37: 1-742⁽⁴⁾ | SEQ ID No. 38 |
| 15771741 (EST) | | | |
| F. graminearum | SEQ ID No. 82: | SEQ ID No. 82: 1-1326 | SEQ ID No. 83 |
| FG00074.1 | 1-1326 | | |
| M. graminicola mg[0281] | SEQ ID No. 39: 1-647 | SEQ ID No. 39: 1-647 | SEQ ID No. 40 |
| (EST) | | | |
| M. graminicola mga0328f | SEQ ID No. 41: 1-560 | SEQ ID No. 41: 1-560 | SEQ ID No. 42 |
| (EST) | | | |
| M. grisea MG03823.3 | SEQ ID No. 43: 1-1254 | SEQ ID No. 43: 1-1254 | SEQ ID No. 44 |
| Ustilago maydis | SEQ ID No. 84: | SEQ ID No. 84: | SEQ ID No. 85 |
| Contig 1.2 | 1-1350 | 1-1350 | |

⁽¹⁾Numbers after SEQ ID Nos. correspond to bases of genomic DNA encoding the protein.
⁽²⁾RNA sequences are given in the sequence listing with Thymidine (T), although it is understood that in vivo Uridine (U) would be present.
⁽³⁾NA one-base deletion at position 690 of the EST (SEQ ID No. 22) is required to give the best predicted cDNA/protein.
⁽⁴⁾Two single base deletions are required to optimise translation.

Bioinformatics analysis was carried out to identify functionally important regions within the fungal 2031 ORs. The 2031 ORs are related to but distinct from the “Old Yellow Enzyme” (OYE) group of yeast enzymes, which also includes ergosterol-binding protein of Candida albicans. Comparison of the 2031 ORs with crystal structures of OYE family proteins identified highly conserved residues responsible for the catalytic function of these enzymes. However, the comparisons also identified seven clusters of residues conserved in 2031 enzymes but not OYE enzymes which flanked the substrate binding site and were therefore implicated in determining substrate specificity (regions 2, 4, 6, 7, 8, 10, and 11 in FIGS. 1 and 2, and Example 4 hereinafter). Four further conserved clusters of residues were identified which, while not predicted to be involved in catalysis, were conserved in 2031 but not OYE and so also distinguish 2031 ORs from OYEs (regions 1, 3, 5, and 9 in FIGS. 1 and 2, and Example 4 hereinafter).

Variants of the above mentioned polynucleotides and proteins are also provided, and are discussed below.

In one embodiment, the protein of the invention may comprise an amino acid sequence substantially as set out and independently selected from regions 1-11 of any of SEQ ID Nos 3, 6, 8, 10, 12, 14, 16, 19, 22, 24, 27, 30, 33, 35, 38, 40, 42, 44, 83 or 85 as given in FIG. 1, or variants thereof. At least one region or motif may be functional.

The polynucleotide of the invention may comprise DNA, such as genomic DNA. The polynucleotide may comprise a sequence substantially as set out and independently selected from regions 1-11 of any of SEQ ID Nos. 1, 4, 7, 9, 11, 13, 15, 17, 20, 23, 25, 28, 31, 34, 36, 39 41, 43, 82 or 84 as given in FIG. 2, or complements, or variants thereof.

Preferably, the polynucleotide encodes a fungal 2031 OR protein which comprises substantially the amino acid sequences SEQ ID Nos 3, 6, 8, 10, 12, 14, 16, 19, 22, 24, 27, 30, 33, 35, 38, 40, 42, 83 or 85 or a variant thereof.

The polynucleotide may comprise RNA, preferably mRNA, preferably spliced mRNA. Preferably, the polynucleotide comprises substantially the sequence shown as SEQ ID Nos 2, 5, 7, 9, 11, 13, 15, 18, 21, 23, 26, 29, 32, 34, 36, 37, 39, 41, 43, 82 or 84 or a complement, or a variant thereof.

Preferably, the protein comprises substantially the sequences SEQ ID Nos. 3, 6, 8, 10, 12, 14, 16, 19, 22, 24, 27, 30, 33, 35, 38, 40, 42, 44, 83 or 85 or a variant thereof.

Preferably, the protein is encoded by the regions of sequences SEQ ID Nos. 1, 4, 7, 9, 11, 13, 15, 17, 20, 23, 25, 26, 28, 29, 31, 34, 36, 39, 41, 43, 82 or 84 as described in FIG. 1. in the column “gDNA/EST” in Table I, or a complement, or a variant thereof.

The polynucleotide may comprise substantially a nucleotide sequence region or motif independently selected from at least one of regions 1-11 from at least one of the sequences SEQ ID Nos. 1, 2, 4, 5, 7, 9, 11, 13, 15, 17, 18, 20, 21, 23, 25, 26, 28, 29, 31, 32, 34, 36, 37, 39, 41, 43, 82 or 84, as given in FIG. 2, or a complement, or a variant thereof.

Preferably, the isolated polynucleotide comprises substantially a nucleotide sequence independently selected from the regions and sequences given in the column “gDNA/EST” in Table I.

Preferably, the protein is encoded by a polynucleotide which polynucleotide comprises substantially a sequence independently selected from at least one of the regions and sequences given in the column “gDNA/EST” in Table I, or a complement or, a variant thereof.

By the term “native amino acid/polynucleotide/protein”, is meant an amino acid, polynucleotide or protein produced naturally from biological sources either in vivo or in vitro.

By the term “synthetic amino acid/polynucleotide/protein”, is meant an amino acid, polynucleotide or protein which has been produced artificially or de novo using a DNA or protein synthesis machine known in the art.

By the term “recombinant amino acid/polynucleotide/protein”, is meant an amino acid, polynucleotide or protein which has been produced using recombinant DNA or protein technology or methodologies which are known to the skilled technician.

The term “variant”, and the terms “substantially the amino acid/polynucleotide/protein sequence” are used herein to refer to related sequences. As discussed below such related sequences are typically homologous to (share percentage identity with) a given sequence, for example over the entire length of the sequence or over a portion of a given length. The related sequence may also be a fragment of the sequence or of a homologous sequence. A variant protein may be encoded by a variant polynucleotide.

By the term “variant”, and the terms “substantially the amino acid/polynucleotide/protein sequence”, we mean that the sequence has at least 30%, preferably 40%, more preferably 50%, and even more preferably, 60% sequence identity with the amino acid/polynucleotide/protein sequences of any one of the sequences referred to. A sequence which is “substantially the amino acid/polynucleotide/peptide sequence” may be the same as the relevant sequence.

Calculation of percentage identities between different amino acid/polynucleotide/protein sequences may be carried out as follows. A multiple alignment is first generated by the ClustalX program (pairwise parameters: gap opening 10.0, gap extension 0.1, protein matrix Gonnet 250, DNA matrix IUB; multiple parameters: gap opening 10.0, gap extension 0.2, delay divergent sequences 30%, DNA transition weight 0.5, negative matrix off, protein matrix gonnet series, DNA weight IUB; Protein gap parameters, residue-specific penalties on, hydrophilic penalties on, hydrophilic residues GPSNDQERK, gap separation distance 4, end gap separation off). The percentage identity is then calculated from the multiple alignment as (N/T)*100, where N is the number of positions at which the two sequences share an identical residue, and T is the total number of positions compared. Alternatively, percentage identity can be calculated as (N/S)*100 where S is the length of the shorter sequence being compared. The amino acid/polynucleotide/protein sequences may be synthesised de novo, or may be native amino acid/polynucleotide/protein sequence, or a derivative thereof.

An amino acid/polynucleotide/protein sequence with a greater identity than 65% to any of the sequences referred to is also envisaged. An amino acid/polynucleotide/protein sequence with a greater identity than 70% to any of the sequences referred to is also envisaged. An amino acid/polynucleotide/protein sequence with a greater identity than 75% to any of the sequences referred to is also envisaged. An amino acid/polynucleotide/protein sequence with a greater identity than 80% to any of the sequences referred to is also envisaged. Preferably, the amino acid/polynucleotide/protein sequence has 85% identity with any of the sequences referred to, more preferably 90% identity, even more preferably 92% identity, even more preferably 95% identity, even more preferably 97% identity, even more preferably 98% identity and, most preferably, 99% identity with any of the referred to sequences.

The above mentioned percentage identities may be measured over the entire length of the original sequence or over a region of 15, 20, 50 or 100 amino acids/bases of the original sequence. In a preferred embodiment percentage identity is measured with reference to SEQ ID No. 3. Preferably the variant protein has at least 40% identity, such as at least 60% or at least 80% identity with SEQ ID No. 3 or a portion of SEQ ID No. 3.

Alternatively, a substantially similar nucleotide sequence will be encoded by a sequence which hybridizes to the sequences shown in SEQ ID Nos. 1, 2, 4, 5, 7, 8, 9, 11, 13, 15, 17, 18, 20, 21, 23, 25, 26, 28, 29, 31, 32, 34, 36, 37, 39, 41, 43, 82 or 84 or their complements under stringent conditions. By stringent conditions, we mean the nucleotide hybridises to filter-bound DNA or RNA in 6× sodium chloride/sodium citrate (SSC) at approximately 45° C. followed by at least one wash in 0.2×SSC/0.1% SDS at approximately 5-65° C. Alternatively, a substantially similar protein may differ by at least 1, but less than 5, 10, 20, 50 or 100 amino acids from the sequences shown in SEQ ID Nos. 3, 6, 8, 10, 12, 14, 16, 19, 22, 24, 27, 30, 33, 35, 38, 40, 42, 44, 83 or 85. Such differences may each be additions, deletions or substitutions.

Due to the degeneracy of the genetic code, it is clear that any nucleic acid sequence could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change.

Other suitable variants are those having homologous nucleotide sequences but comprising all, or portions of, sequence which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. Certain organisms, including Candida are known to use non-standard codons compared to those used in the majority of eukaryotes. Any comparisons of polynucleotides and proteins from such organisms with the sequences given here should take these differences into account.

In accurate alignment of protein or DNA sequences the trade-off between optimal matching of sequences and the introduction of gaps to obtain such a match is important. In the case of proteins, the means by which matches are scored is also of significance. The family of PAM matrices (e.g., Dayhoff, M. et al., 1978, Atlas of protein sequence and structure, Natl. Biomed. Res. Found.) and BLOSUM matrices quantitate the nature and likelihood of conservative substitutions and are used in multiple alignment algorithms, although other, equally applicable matrices will be known to those skilled in the art. The popular multiple alignment program ClustalW, and its windows version ClustalX (Thompson et al., 1994, Nucleic Acids Research, 22, 4673-4680; Thompson et al., 1997, Nucleic Acids Research, 24, 4876-4882) are efficient ways to generate multiple alignments of proteins and DNA.

Use of the Align program is also preferred (Hepperle, D., 2001: Multicolor Sequence Alignment Editor. Institute of Freshwater Ecology and Inland Fisheries, 16775 Stechlin, Germany), although others, such as JalView or Cinema are also suitable.

Calculation of percentage identities between proteins occurs during the generation of multiple alignments by Clustal. However, these values need to be recalculated if the alignment has been manually improved, or for the deliberate comparison of two sequences. Programs that calculate this value for pairs of protein sequences within an alignment include PROTDIST within the PHYLIP phylogeny package (Felsenstein) using the “Similarity Table” option as the model for amino acid substitution (P). For DNA/RNA, an identical option exists within the DNADIST program of PHYLIP.

Other modifications in protein sequences are also envisaged and within the scope of the claimed invention, i.e. those which occur during or after translation, e.g. by acetylation, amidation, carboxylation, phosphorylation, proteolytic cleavage or linkage to a ligand.

The term “variant”, and the terms “substantially the amino acid/polynucleotide/protein sequence” also include a fragment of the relevant polynucleotide or protein sequences, including a fragment of the homologous sequences (which have percentage identity to a specified sequence) referred to above. A polynucleotide fragment will typically comprise at least 10 bases, such as at least 20, 30, 50, 100, 200, 500 or 1000 bases. A protein fragment will typically comprise at least 10 amino acids, such as at least 20, 30, 50, 80, 100, 150, 200, 300, 400 or 500 amino acids. The fragments may lack at least 3 amino acids, such as at least 10, 20 or 30 amino acids of the amino acids from either end of the protein.

The invention provides a method of screening which may be used to identify modulators of 2031 OR proteins or polynucleotides, such as inhibitors of expression or activity of the proteins or polynucleotides of the invention. In one embodiment of the method a candidate substance is contacted with a protein or polynucleotide of the invention and whether or not the candidate substance binds or modulates the protein or polynucleotide is determined.

The modulator may promote (agonise) or inhibit (antagonise) the activity of the protein. A therapeutic modulator (against fungal infection) will inhibit the expression or activity of protein or polynucleotide of the invention.

The method may be carried out in vitro (inside or outside a cell) or in vivo. In one embodiment the method is carried out on a cell, or cell culture cell extract. The cell may or may not be a cell in which the polynucleotide or protein is naturally present. The cell may or may not be a fungal cell, or may or may not be a cell of any of the fungi mentioned herein. The protein or polynucleotide may be present in a non-cellular form in the method, thus the protein may be in the form of a recombinant protein purified from a cell.

Any suitable binding or activity assay may be used. Methods which determine whether a candidate substance is able to bind the protein or polynucleotide may comprise providing the protein or polynucleotide to a candidate substance and determining whether binding occurs, for example by measuring the amount of the candidate substance which binds the protein or polynucleotide. The binding may be determined by measuring a characteristic of the protein or polynucleotide that changes upon binding, such as spectroscopic changes. The binding may be determined by measuring reaction substrate or product levels in the presence and absence of the candidate and comparing the levels.

The assay format may be a ‘band shift’ system. This involves determining whether a test candidate advances or retards the protein or polynucleotide on gel electrophoresis relative to the absence of the compound.

The method may be a competitive binding method. This determines whether the candidate is able to inhibit the binding of the protein or polynucleotide to an agent which is known to bind to the protein or polynucleotide, such as an antibody specific for the protein, or a substrate of the protein.

Whether or not a candidate substance modulates the activity of the protein may be determined by providing the candidate substance to the protein under conditions that permit activity of the protein, and determining whether the candidate substance is able to modulate the activity of the product.

The activity which is measured may be any of the activities of the protein of the invention mentioned herein, such as oxidoreductase activity. In one embodiment the screening method comprising carrying out a redox reaction in the presence and absence of the candidate substance to determine whether the candidate substance inhibits the oxidoreductase activity of the protein of the invention, wherein the redox reaction is carried out by contacting said protein with NADH or NADPH; and an electron acceptor, under conditions in which in the absence of the candidate substance the protein catalyses reduction of the electron acceptor.

In a preferred embodiment the inhibition of the redox reaction is measured by detecting the amount of NADH or NADPH oxidation, for example by measuring the generation of the oxidised forms of NADH and NADPH spectroscopically. This can be done by measurement at 340 nm (see Example 7).

Alternatively, a suitable colourimetric oxidoreductase substrate may be used to measure inhibition, such as methylene blue, phenazine methosulphate or 2,6-dichlorophenolindophenol.

Suitable candidate substances which can tested in the above methods include antibody products (for example, monoclonal and polyclonal antibodies, single chain antibodies, chimeric antibodies and CDR-grafted antibodies). Furthermore, combinatorial libraries, defined chemical identities, peptide and peptide mimetics, oligonucleotides and natural product libraries, such as display libraries (e.g. phage display libraries) may also be tested. The candidate substances may be chemical compounds. Batches of the candidate substances may be used in an initial screen of, for example, ten substances per reaction, and the substances from batches which show inhibition tested individually.

According to a further aspect of the present invention, there is provided a polynucleotide or protein of the invention for use as a medicament or in diagnosis.

The polynucleotide or protein may be modified prior to use, preferably to produce a derivative or variant thereof. The polynucleotide or protein may be derivatised. The protein may be modified by epitope tagging, addition of fusion partners or purification tags such as glutathione S-transferase, multiple histidines or maltose binding protein, addition of green fluorescent protein, covalent attachment of molecules including biotin or fluorescent tags, incorporation of selenomethionine, inclusion or attachment of radioisotopes or fluorescent/non-fluorescent lanthanide chelates. The polynucleotide may be modified by methylation or attachment of digoxygenin (DIG) or by addition of sequence encoding the above tags, proteins or epitopes.

Preferably, the medicament is adapted to retard or prevent a fungal infection. The fungal infection may be in human, animal or plant. The polynucleotide or protein may be used for the development of a drug. The polynucleotide or protein may be used in, or for the generation of, a molecular model of said polynucleotide or said protein.

According to a further aspect of the present invention, there is provided use of a polynucleotide or protein of the invention for the preparation of a medicament for the treatment of a fungal infection.

The polynucleotide or protein may be modified prior to use, preferably to produce a derivative or variant thereof. The polynucleotide or protein may be derivatised. The polynucleotide or protein may not be modified or derivatised.

Preferably, the medicament is adapted to retard or prevent a fungal infection. The treatment may comprise retarding or preventing fungal infection. Preferably, the drug and/or medicament comprises an inhibitor, preferably a 2031 OR inhibitor. Preferably, the drug or medicament is adapted to inhibit expression and/or activity of the polynucleotide or a fragment thereof, and/or the function of the protein or a fragment thereof.

Preferably, the fungal infection comprises an infection by a fungus, more preferably an Ascomycete, and even more preferably, an organism selected from the genera Aspergillus, Blumeria; Candida; Colletotrichium; Cryptococcus; Encephalitozoon; Fusarium; Leptosphaeria; Magnaporthe; Mycosphaerella; Neurospora, Phytophthora; Plasmopara; Pneumocystis; Pyricularia; Pythium; Puccinia; Rhizoctonia; Schizosaccharomyces, Trichophyton, and Ustilago.

Preferably, the fungal infection comprises an infection by an organism selected from the genera Aspergillus, Candida, Colletotrichium, Fusarium, Magnaporthe, Mycosphaerella and Ustilago.

Preferably, the fungal infection comprises an infection by an organism selected from the species Aspergillus flavus; Aspergillus fumigatus; Aspergillus nidulans; Aspergillus niger; Aspergillus parasiticus; Aspergillus terreus, Blumeria graminis; Candida albicans; Candida cruzei; Candida glabrata; Candida parapsilosis; Candida tropicalis; Colletotrichium trifolii; Cryptococcus neoformans; Encephalitozoon cuniculi; Fusarium graminarium; Fusarium solani; Fusarium sporotrichoides; Leptosphaeria nodorum; Magnaporthe grisea; Mycosphaerella graminicola; Phytophthora capsici; Phytophthora infestans; Plasmopara viticola; Pneumocystis jiroveci; Puccinia coronata; Puccinia graminis; Pyricularia oryzae; Pythium ultimum; Rhizoctonia solani; Trichophyton interdigitale; Trichophyton rubrum; and Ustilago maydis.

Preferably, the fungal infection comprises an infection by an organism selected from the species Aspergillus fumigatus; Aspergillus nidulans, Candida albicans, Colletotrichium trifolii, Fusarium graminearum, Fusarium sporotrichoides, Magnaporthe grisea, Mycosphaerella graminicola and Ustilago maydis.

According to another aspect of the present invention, there is provided a method of detecting the presence of a fungal infection in an individual, said method comprising:

(i) obtaining a sample from an organism; and

(ii) detecting in the said sample the presence of a polynucleotide or protein of the invention.

The individual may be a person (human) or animal (such as a mammal or bird) or a plant. The fungal infection may arise from infection with an organism selected from the genera Aspergillus; Blumeria; Candida; Colletotrichium; Cryptococcus; Encephalitozoon; Fusarium; Leptosphaeria; Magnaporthe; Mycosphaerella; Phytophthora; Plasmopara; Pneumocystis; Pyricularia; Pythium, Puccinia; Rhizoctonia; Trichophyton; and Ustilago

The fungal infection may arise from infection with an organism selected from the species Aspergillus flavus; Aspergillus fumigatus; Aspergillus nidulans; Aspergillus niger; Aspergillus parasiticus; Aspergillus terreus; Blumeria graminis; Candida albicans; Candida cruzei; Candida glabrata; Candida parapsilosis; Candida tropicalis; Colletotrichium trifolii, Cryptococcus neoformans; Encephalitozoon cuniculi; Fusarium graminarium, Fusarium solani; Fusarium sporotrichoides; Leptosphaeria nodorum; Magnaporthe grisea; Mycosphaerella graminicola; Phytophthora capsici, Phytophthora infestans; Plasmopara viticola; Pneumocystis jiroveci; Puccinia coronata; Puccinia graminis; Pyricularia oryzae; Pythium ultimum, Rhizoctonia solani; Trichophyton interdigitale; Trichophyton rubrum; and Ustilago maydis.

Preferably, the sample comprises a biological sample which, preferably, comprises nucleic acid and/or protein. In one embodiment of the method the nucleic acid or protein is purified (at least partially) from the sample before the detection is performed.

Where the organism is Aspergillus fumigatus, Aspergillus nidulans or Aspergillus niger, the sample may comprise sputum, bronchoalveloar lavage, urine, respiratory specimens, endotracheal aspirates, sterile specimens obtained by an invasive procedure such as vitreous tap, tympanocentesis, brain biopsy or aspiration, nasal or sinus specimens, blood, tissue or autopsy.

Where the organism is Magnaporthe grisea the sample may comprise rice leaf or rice stem.

Preferably, said detecting of the presence in the said sample of a polynucleotide as defined by the first or third aspect comprises use of at least one oligonucleotide pair adapted to be used for amplification of DNA, preferably genomic, more preferably, fungal genomic DNA. The amplification may be PCR amplification.

Preferably, the PCR amplification employs at least one primer pair comprising a polynucleotide selected from the group consisting of:

Aspergillus fumigatus; SEQ ID Nos 67 and 68 for SEQ ID No. 1; SEQ ID Nos 69 and 70 for SEQ ID No. 4; and SEQ ID Nos 71 and 72 for SEQ ID No. 7.

Candida albicans; SEQ ID Nos 73 and 74 for SEQ ID No. 11.

Magnaporthe grisea; SEQ ID Nos 75 and 76 for SEQ ID No. 20.

Preferably, said detecting comprises subjecting the amplified DNA to size analysis, preferably, electrophoresis and, preferably, comparing the results to a positive control and, preferably, a negative control. Said detecting may also comprise sequencing of the amplified DNA to demonstrate the correct sequence.

Preferably, said detecting of the presence in the said sample of a protein comprises use of a monoclonal or polyclonal antibody directed to part or all of the protein of the invention.

According to a further aspect of the present invention, there is provided a recombinant DNA molecule or vector comprising a polynucleotide of the invention.

The recombinant DNA molecule or vector may comprise an expression cassette. Preferably, the recombinant DNA molecule or vector comprises an expression vector. Preferably, the polynucleotide sequence is operatively linked to an expression control sequence. A suitable control sequence may comprise a promoter, an enhancer etc.

According to another aspect of the present invention, there is provided a cell containing a polynucleotide, recombinant DNA molecule or vector of the invention.

The cell may be transformed or transfected with the polynucleotide, recombinant DNA molecule or vector by suitable means. Preferably, the cell produces a recombinant protein of the invention.

The invention also provides an organism which is transgenic for the polynucleotide of the invention (whose cells may be the same as the cells of the invention mentioned herein). Such an organism is typically a fungus, such as any genera or species of fungus mentioned herein. The organism may be microorganism, such as a bacterium, virus or yeast. The organism may be a plant, animal (including birds and mammals), such as any of the animals mentioned herein.

The organism may be produced by introduction of the polynucleotide of the invention into a cell of the organism, and in the case of a multicellular organism allowing the cell to grow into a whole organism.

According to a further aspect of the present invention, there is provided a cell in which a native polynucleotide or protein of the invention protein is non-functional and/or inhibited. The cell may be of, or present in, a multicellular organism.

The cell may be a mutant cell. The cell is typically a fungal cell, such as of any genera or species of fungus mentioned herein. A preferred means of generating the cell is to modify the polynucleotide of the invention, such that the polynucleotide is non-functional. This modification may be to cause a mutation, which disrupts the expression or function of a gene product. Such mutations may be to the nucleic acid sequences that act as 5′ or 3′ regulatory sequences for the polynucleotide, or may be a mutation introduced into the coding sequence of the polynucleotide. Functional deletion of the polynucleotide may be, for example, by mutation of the polynucleotide in the form of nucleotide substitution, addition or, preferably, nucleotide deletion.

The polynucleotide may be made non-functional and/or inhibited by:

(i) shifting the reading frame of the coding sequence of the polynucleotide;

(ii) adding, substituting or deleting amino acids in the protein encoded by the polynucleotide; or

(iii) partially or entirely deleting the DNA coding for the polynucleotide and/or the upstream and downstream regulatory sequences associated with the polynucleotide.

(iv) inserting DNA into the coding or non-coding regions.

A preferred means of introducing a mutation into a polynucleotide is to utilize molecular biology techniques specifically to target the polynucleotide which is to be mutated. Mutations may be induced using a DNA molecule. A most preferred means of introducing a mutation is to use a DNA molecule that has been especially prepared such that homologous recombination occurs between the target polynucleotide and the DNA molecule. When this is the case, the DNA molecule, which may be double stranded, may contain base sequences similar or identical to the target polynucleotide to allow the DNA molecule to hybridize to (and subsequently recombine with) the target.

It is also possible to provide a cell in which the polynucleotide is non-functional and/or inhibited without introducing a mutation into the gene or its regulatory regions. This may be done by using specific inhibitors. Examples of such inhibitors include agents that prevent transcription of the polynucleotide, or prevent translation, expression or disrupt post-translational modification. Alternatively, the inhibitor may be an agent that increases degradation of the gene product (e.g. a specific proteolytic enzyme). Equally, the inhibitor may be an agent which prevents the polynucleotide product from functioning, such as neutralizing antibodies (for instance an anti-2031 OR antibody). The inhibitor may also be an antisense oligonucleotide, or any synthetic chemical capable of inhibiting expression of the gene or the stability and/or function of the protein. The inhibitor may also be a protein which interacts with the 2031 OR to prevent its function. The inhibitor may also be an RNA molecule which causes inhibition by RNA interference. In one embodiment the antisense polynucleotide or RNA molecule which causes RNA interference are examples of polynucleotides of the invention.

According to a further aspect, there is provided an antibody exhibiting immunospecificity for a protein of the invention. The antibody may be used as a diagnostic reagent.

The antibody may be monoclonal or polyclonal, and may be raised in mouse, rat, rabbit, chicken, turkey, horse, goat or donkey. The antibody may be raised against one or all of the proteins together, or may be raised against proteolytic or recombinant fragments.

For the purposes of this invention, the term “antibody”, unless specified to the contrary, includes fragments which bind a protein of the invention. Such fragments include Fv, F(ab′) and F(ab′)₂ fragments, as well as single chain antibodies. Furthermore, the antibodies and fragment thereof may be chimeric antibodies, CDR-grafted antibodies or humanised antibodies.

### Administration

The formulation of any of the therapeutic substances (e.g. proteins, polynucleotides or modulators) mentioned herein will depend upon factors such as the nature of the substance and the condition to be treated. Any such substance may be administered in a variety of dosage forms. It may be administered orally (e.g. as tablets, troches, lozenges, aqueous or oily suspensions, dispersible powders or granules), parenterally, subcutaneously, intravenously, intramuscularly, intrasternally, transdermally or by infusion techniques. The substance may also be administered as suppositories. A physician will be able to determine the required route of administration for each particular patient.

Typically the substance is formulated for use with a pharmaceutically acceptable carrier or diluent. The pharmaceutical carrier or diluent may be, for example, an isotonic solution. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film coating processes.

Liquid dispersions for oral administration may be syrups, emulsions and suspensions. The syrups may contain as carriers, for example, saccharose or saccharose with glycerine and/or mannitol and/or sorbitol. Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for intravenous or infusions may contain as carrier, for example, sterile water or preferably they may be in the form of sterile, aqueous, isotonic saline solutions.

A therapeutically effective non-toxic amount of substance is administered. The dose may be determined according to various parameters, especially according to the substance used; the age, weight and condition of the patient to be treated; the route of administration; and the required regimen. Again, a physician will be able to determine the required route of administration and dosage for any particular patient. A typical daily dose is from about 0.1 to 50 mg per kg, preferably from about 0.1 mg/kg to 10 mg/kg of body weight, according to the activity of the specific inhibitor, the age, weight and conditions of the subject to be treated, the type and severity of the disease and the frequency and route of administration. Preferably, daily dosage levels are from 5 mg to 2 g.

### Agricultural Use

Modulators identified by the method of the invention may be administered to plants in order to prevent or treat fungal infections. The modulators are normally applied in the form of compositions together with one or more agriculturally acceptable carriers or diluents and can be applied to the crop area or plant to be treated, simultaneously or in succession with further compounds.

The modulators of the invention can be applied together with carriers, surfactants or application-promoting adjuvants customarily employed in the art of formulation. Suitable carriers and diluents correspond to substances ordinarily employed in formulation technology, e.g. natural or regenerated mineral substances, solvents, dispersants, wetting agents, tackifiers, binders or fertilizers.

A preferred method of applying the modulators of the present invention or an agrochemical composition which contains them is leaf application. The number of applications and the rate of application depend on the intensity of infection by the fungus. However, the active ingredients can also penetrate the plant through the roots via the soil (systemic action) by impregnating the locus of the plant with a liquid composition, or by applying the compounds in solid form to the soil, e.g. in granular form (soil application). The active ingredients may also be applied to seeds (coating) by impregnating the seeds either with a liquid formulation containing active ingredients, or coating them with a solid formulation. In special cases, further types of application are also possible, for example, selective treatment of the plant stems or buds.

The active ingredients are used in unmodified form or, preferably, together with the adjuvants conventionally employed in the art of formulation, and are therefore formulated in known manner to emulsifiable concentrates, coatable pastes, directly sprayable or dilutable solutions, dilute emulsions, wettable powders, soluble powders, dusts, granulates, and also encapsulations, for example, in polymer substances. Like the nature of the compositions, the methods of application, such as spraying, atomizing, dusting, scattering or pouring, are chosen in accordance with the intended objectives and the prevailing circumstances. Advantageous rates of application are normally from 50 g to 5 kg of active ingredient (a.i.) per hectare (“ha”, approximately 2.471 acres), preferably from 100 g to 2 kg a.i./ha, most preferably from 200 g to 500 g a.i./ha.

The formulations, compositions or preparations containing the active ingredients and, where appropriate, a solid or liquid adjuvant, are prepared in known manner, for example by homogeneously mixing and/or grinding active ingredients with extenders, for example solvents, solid carriers and, where appropriate, surface-active compounds (surfactants).

Suitable solvents include aromatic hydrocarbons, preferably the fractions having 8 to 12 carbon atoms, for example, xylene mixtures or substituted naphthalenes, phthalates such as dibutyl phthalate or dioctyl phthalate, aliphatic hydrocarbons such as cyclohexane or paraffins, alcohols and glycols and their ethers and esters, such as ethanol, ethylene glycol, monomethyl or monoethyl ether, ketones such as cyclohexanone, strongly polar solvents such as N-methyl-2-pyrrolidone, dimethyl sulfoxide or dimethyl formamide, as well as epoxidized vegetable oils such as epoxidized coconut oil or soybean oil; or water.

The solid carriers used e.g. for dusts and dispersible powders, are normally natural mineral fillers such as calcite, talcum, kaolin, montmorillonite or attapulgite. In order to improve the physical properties it is also possible to add highly dispersed silicic acid or highly dispersed absorbent polymers. Suitable granulated adsorptive carriers are porous types, for example pumice, broken brick, sepiolite or bentonite; and suitable nonsorbent carriers are materials such as calcite or sand. In addition, a great number of pregranulated materials of inorganic or organic nature can be used, e.g. especially dolomite or pulverized plant residues.

Depending on the nature of the active ingredient to be used in the formulation, suitable surface-active compounds are nonionic, cationic and/or anionic surfactants having good emulsifying, dispersing and wetting properties. The term “surfactants” will also be understood as comprising mixtures of surfactants.

Suitable anionic surfactants can be both water-soluble soaps and water-soluble synthetic surface-active compounds. Suitable soaps are the alkali metal salts, alkaline earth metal salts or unsubstituted or substituted ammonium salts of higher fatty acids (chains of 10 to 22 carbon atoms), for example the sodium or potassium salts of oleic or stearic acid, or of natural fatty acid mixtures which can be obtained for example from coconut oil or tallow oil. The fatty acid methyltaurin salts may also be used.

More frequently, however, so-called synthetic surfactants are used, especially fatty sulfonates, fatty sulfates, sulfonated benzimidazole derivatives or alkylarylsulfonates. The fatty sulfonates or sulfates are usually in the form of alkali metal salts, alkaline earth metal salts or unsubstituted or substituted ammoniums salts and have a 8 to 22 carbon alkyl radical which also includes the alkyl moiety of alkyl radicals, for example, the sodium or calcium salt of lignonsulfonic acid, of dodecylsulfate or of a mixture of fatty alcohol sulfates obtained from natural fatty acids. These compounds also comprise the salts of sulfuric acid esters and sulfonic acids of fatty alcohol/ethylene oxide adducts. The sulfonated benzimidazole derivatives preferably contain 2 sulfonic acid groups and one fatty acid radical containing 8 to 22 carbon atoms. Examples of alkylarylsulfonates are the sodium, calcium or triethanolamine salts of dodecylbenzenesulfonic acid, dibutylnaphthalenesulfonic acid, or of a naphthalenesulfonic acid/formaldehyde condensation product. Also suitable are corresponding phosphates, e.g. salts of the phosphoric acid ester of an adduct of p-nonylphenol with 4 to 14 moles of ethylene oxide.

Non-ionic surfactants are preferably polyglycol ether derivatives of aliphatic or cycloaliphatic alcohols, or saturated or unsaturated fatty acids and alkylphenols, said derivatives containing 3 to 30 glycol ether groups and 8 to 20 carbon atoms in the (aliphatic) hydrocarbon moiety and 6 to 18 carbon atoms in the alkyl moiety of the alkylphenols.

Further suitable non-ionic surfactants are the water-soluble adducts of polyethylene oxide with polypropylene glycol, ethylenediamine propylene glycol and alkylpolypropylene glycol containing 1 to 10 carbon atoms in the alkyl chain, which adducts contain 20 to 250 ethylene glycol ether groups and 10 to 100 propylene glycol ether groups. These compounds usually contain 1 to 5 ethylene glycol units per propylene glycol unit.

Representative examples of non-ionic surfactants are nonylphenolpolyethoxyethanols, castor oil polyglycol ethers, polypropylene/polyethylene oxide adducts, tributylphenoxypolyethoxyethanol, polyethylene glycol and octylphenoxyethoxyethanol. Fatty acid esters of polyoxyethylene sorbitan and polyoxyethylene sorbitan trioleate are also suitable non-ionic surfactants.

Cationic surfactants are preferably quaternary ammonium salts which have, as N-substituent, at least one C₈-C₂₂ alkyl radical and, as further substituents, lower unsubstituted or halogenated alkyl, benzyl or lower hydroxyalkyl radicals. The salts are preferably in the form of halides, methylsulfates or ethylsulfates, e.g. stearyltrimethylammonium chloride or benzyldi(2-chloroethyl)ethylammonium bromide.

The surfactants customarily employed in the art of formulation are described, for example, in “McCutcheon's Detergents and Emulsifiers Annual”, MC Publishing Corp. Ringwood, N.J., 1979, and Sisely and Wood, “Encyclopaedia of Surface Active Agents,” Chemical Publishing Co., Inc. New York, 1980.

The agrochemical compositions usually contain from about 0.1 to about 99% preferably about 0.1 to about 95%, and most preferably from about 3 to about 90% of the active ingredient, from about 1 to about 99.9%, preferably from about 1 to 99%, and most preferably from about 5 to about 95% of a solid or liquid adjuvant, and from about 0 to about 25%, preferably about 0.1 to about 25%, and most preferably from about 0.1 to about 20% of a surfactant. Whereas commercial products are preferably formulated as concentrates, the end user will normally employ dilute formulations.

All of the features described herein may be combined with any of the above aspects, in any combination.

### EXAMPLES

### Example 1

### Identification of an Essential Gene in Aspergillus fumigatus

An essential region of the A. fumigatus genome was identified using the mycobank technology as described in patent WO00177295A1 with the following modifications:

### Re-Haploidisation (Section 1.6):

P24 lines 11-18: Conidia (A. fumigatus) were collected from a stable diploid transformant colony and approximately 3×10⁴ spores were used to inoculate 1 ml of SAB broth containing 1 mg/ml FPA. This culture was incubated with shaking (200 rpm) at 37° C. for 20 hours. 100 μl of the culture was spread onto complete media containing 0.2 mg/ml FPA and incubated at 37° C. for 3 days or until rapidly growing sectors emerged. Conidia were collected from each sector and plated onto nitrate, nitrite and hypoxanthine media and the nitrogen utilisation profiles of the resulting conidia assessed. Colonies with the nitrogen utilisation profiles of the parental strains indicated breakdown of the diploid to a haploid. 44 haploid sectors were isolated from transformant 2031. None of the haploids isolated were hygromycin resistant indicating the insertion of the hph gene into a portion of the genome required for function.

### Transformation (Section 1.7):

P25 line 9: Plasmid pAN7-1 linearised with HindIII was used as the transforming vector. PAN7-1 carries the hph gene which confers hygromycin resistance.

P25 lines 17-20:1 ml of cold YED was added to the cuvette and incubated at 37° C. for 1 h. Aliquots were spread on selective agar (complete media with 250 μg/ml hygromycin). Colonies growing on selective media were deemed putative transformants.

The point of insertion was identified using the plasmid rescue method outlined on page 31 lines 5-17. The insertion site was confirmed by employing PCR: Using the sequence obtained from plasmid rescue data a primer was designed within the sequence of pAN7-1 and a complementary primer was designed within the predicted sequence near the point of insertion. Genomic DNA isolated from the diploid 2031 was used as a template.

The resulting DNA sequence (experiment 2031, with 175 bases of upstream pAN7.1 sequence removed) corresponds to the gDNA sequence immediately downstream of the insertion site and is given as SEQ ID No. 45.

### Example 2

### Characterisation of the Essential Gene

### 2.1 Genome Analysis

The TIGR A. fumigatus database (TIGR) was searched (blastn) with the sequence SEQ ID No. 45, identified in Example 1 above, and a match to contig 4798 (Eval 4.6e-148) was identified. The appropriate region of the contig sequence was down-loaded from www.tigr.org and gene predictions carried out using Genscan (Settings; organism=vertebrate; Suboptimal exon cutoff=1.00).

The ab initio prediction of genes from genomes is known to be an inaccurate process (Burset, M. and Guigó, 1996, Genomics, 34, 353-367) and this is particularly so when the programs used have not been specifically trained for the genome under examination (as is the case here). It is therefore necessary to carefully examine the predictions, to compare any predicted genes with any homologous proteins, and to exploit the operative's knowledge of fungal gene structure, and thus to arrive at an informed prediction. The predicted genes were therefore compared with similar sequences using blastp, the multiple alignment program ClustalX (Thompson et al., 1997, Nucleic Acids Research, 24:4876-4882), and the alignment editor/viewer Align (Hepperle, D., 2001: Multicolor Sequence Alignment Editor. Institute of Freshwater Ecology and Inland Fisheries, 16775 Stechlin, Germany). Gene structures were visualised and modified using Artemis (Rutherford et al., 2000, Bioinformatics 16, 944-945).

The gene adjacent to the insertion site corresponded to bases 299-469 (exon 1) and bases 520-1618 (exon 2) of the genomic sequence given as SEQ ID No. 1. The protein sequence for the gene is given as SEQ ID No. 3. The insertion site was 735 bases upstream of the 5′ ATG start of the gene.

Searches of the protein databases at blast.genome.adjp showed that protein SEQ ID No. 3 is a member of the NADH-dependent flavin oxidoreductase family. This protein is henceforth referred to as 2031 oxidoreductase (2031 OR; having come from mycobank experiment 2031). Other 2031 OR-like proteins were also identified (see Example 4.1). The NADH-dependent flavin oxidoreductase family also includes Old Yellow Enzyme (OYE), from S. cerevisiae and other fungi, although 2031 ORs can be distinguished from OYEs.

Referring to FIG. 1, there is shown a multiple alignment of the 2031 OR amino acid sequence from A. fumigatus along with related ORs from other fungi and bacteria (see also Example 4). Regions 1-11 refer to amino acids conserved between ORs.

Fungal 2031 ORs are given by: SEQ ID Nos. 3, 6 and 8, A. fumigatus; SEQ ID No. 10, A. nidulans; SEQ ID Nos. 12 and 14, C. albicans; SEQ ID Nos. 16 and 19, N. crassa; SEQ ID Nos 22 and 44, M. grisea; SEQ ID No. 24, (NP_{—}595868), S. pombe; SEQ ID No. 27, C. trifolii; SEQ ID Nos. 30, 33 and 35, F. sporotrichioides; SEQ ID Nos. 38 and 83, F. graminearum SEQ ID Nos. 40 and 42, M. graminicola; SEQ ID No. 85, U. maydis.

Bacterial ORs resembling 2031 are: T44612 (Pseudomonas putida), SEQ ID No. 86; NP_{—}625402 (Streptomyces coelicolor), SEQ ID No. 87; NP_{—}295913 (Deinococcus radiodurans), SEQ ID No. 88; AF320254 (Azoarcus evansii, SEQ ID No. 89.

Fungal ORs similar to the Old Yellow Enzyme family (originally identified in S. cerevisiae): A. fumigatus, Af4875 and Af4961, SEQ ID Nos. 90 and 91 respectively; C. albicans, Ca2460 and A36990, SEQ ID Nos. 92 and 93 respectively; N. crassa, Nc4452, SEQ ID No. 94; S. cerevisiae, OYE1, OYE2 and OYE3, SEQ ID Nos. 95-97 respectively.

Details of the sequence searches that identified the ORs other than SEQ ID No. 3, and methods for the construction of multiple alignments are given in Example 4 hereinafter.

Referring to FIG. 2, there is shown a multiple alignment of the nucleotide sequence of 2031 OR from A. fumigatus along with related 2031 ORs from other fungi and bacteria (see also Example 4). Regions 1-11 refer to amino acids conserved between 2031 ORs at the amino acid level. Fungal 2031 ORs are given by SEQ ID No.: SEQ ID Nos. 1, 2, 4, 5, and 7, A. fumigatus; SEQ ID No. 9, A. nidulans; SEQ ID Nos. 11 and 13, C. albicans; SEQ ID Nos. 15, 17 and 18, N. crassa; SEQ ID Nos. 20, 21 and 43, M. grisea; SEQ ID No. 23 (NP_{—}595868), S. pombe; SEQ ID Nos. 25 and 26, C. trifolii; SEQ ID Nos. 28, 29, 31, 32 and 34, F. sporotrichioides; SEQ ID Nos. 36, 37 and 82, F. graminearum; SEQ ID Nos. 39 and 41, M. graminicola; SEQ ID No. 84, U. maydis.

Details of the sequence searches that identified the ORs, and methods for the construction of multiple alignments are given in Example 4 hereinafter.

### 2.2 Genomic Sequencing of Genes

Following the above bioinformatic analyses, the genomic sequences of 2031 OR was experimentally determined.

2.2.1 Bacterial and Fungal Strains

For bacterial cloning, E. coli strains Top10 (Invitrogen) and select96 (Promega) were used in accordance with manufacturers' instructions.

A. fumigatus clinical isolate AF293 (ref. No. NCPF7367; available to the public from the NCPF repository; Bristol, U.K.); the CBS repository (Belgium) or from Dr. David Denning's clinical isolate culture collection, Hope Hospital, Salford. U.K.) is the preferred strain according to the present invention. AF293 was isolated in 1993 from the lung biopsy of a patient with invasive aspergillosis and aplastic anaemia. It was donated by Shrewsbury PHLS.

2.2.2 Purification of A. fumigatus Genomic DNA

To obtain mycelial material for genomic DNA isolation, approximately 10⁷ A. fumigatus conidia were inoculated in 50 ml of Vogel's minimal medium and incubated with shaking at 200 rpm until late exponential phase (18-24 h) at 37° C. Mycelium was dried down onto Whatmann 54 paper using a Buckner funnel and a side-arm flask attached to a vacuum pump and washed with PBS/Tween. At this point, the mycelium could be freeze-dried for extraction at a later date.

The mycelium (fresh or freeze dried) was ground to a powder using liquid nitrogen in a −20° C. cooled mortar. The ground biomass was transferred to 50 ml tubes on ice up to the 10 ml mark. An equal volume of extraction buffer (0.7 M NaCl; 0.1 M Na₂SO₃; 0.1 M Tris-HCl pH 7.5; 0.05 M EDTA; 1% (w/v) SDS; pre-warmed to 65° C.) was then added to each tube, mixed thoroughly with a pipette tip and incubated at 65° C. for 20 minutes in a water bath. A volume of chloroform/isoamyl alcohol (24:1) equivalent to the volume of the original biomass was then added to each tube, tubes were mixed thoroughly and incubated on ice for 30 min. Tubes were then centrifuged at 3,500×g for 30 min and the aqueous phase carefully transferred to fresh 50 ml tubes without disturbing the interface.

An equal volume of chloroform/isoamyl alcohol (24:1) was added, the tubes vortexed and incubated on ice for 15 minutes. Tubes were then spun at 3,500×g for 15 minutes. After this spin, if large amounts of precipitate were still present, the supernatant was removed and the chloroform:isoamyl alcohol step repeated. The supernatant was removed and placed in clean sterile Oak Ridge tubes. An equal volume of isopropanol was added and mixed gently. Tubes were incubated at room temperature for at least 15 minutes. Tubes were then centrifuged at 3,030×g for 10 minutes at 4° C. to pellet the DNA. The supernatant was removed and the pellet allowed to air dry for 10-25 minutes. The pellet was suspended in 2 ml sterile water. 1 ml of 7.5 M ammonium acetate was added, mixed and incubated on ice for 1 hour. Tubes were centrifuged at 12,000×g for 30 min, the supernatants transferred to a fresh tube and 0.54 volumes of isopropanol were added, mixed and incubated at room temperature for at least 15 minutes. Tubes were then centrifuged at 5,930×g for 10 min, the supernatant was removed and the pellet washed in 1 ml of 70% ethanol. Tubes were centrifuged at 5,930×g for 10 min and all the ethanol was removed. The pellet was air dried for 20-30 minutes at room temperature and suspended in 0.5-1.0 ml of TE (10 mM Tris-HCl pH 7.5; 1 mM EDTA) Finally, the DNA was treated with RNase A (5 μl of 1 mg/ml stock).

2.2.3 PCR Reactions

Primers were designed to the upstream and downstream regions of the A. fumigatus AF293 2031 OR; cloning primer pair SEQ ID Nos. 46 (Ox9_for) and 47 (Ox10_rev). The following reagents and conditions were used:

PCR Master Mix

| | |
|---|---|
| 10x high fidelity PCR buffer | 5 μl |
| dNTP (clontech: 10 mM) | 1 μl |
| nH₂O | 39 μl |
| Pfu Ultra Polmerase (2.5 U/μl) | 1 μl |
| Forward primer (Ox9_for: 10 pmol/μl stock) | 1 μl |
| Reverse primer (Ox10_rev: 10 pmol/μl stock) | 1 μl |
| gDNA (1:30 dilution of stock) | 2 μl |

PCR Cycle

| | | |
|---|---|---|
| 1) 95° C. | 2 | min |
| 2) 95° C. | 30 | sec |
| 3) 54° C. | 30 | sec |
| 4) 72° C. | 2 | min |
| 5) 72° C. | 10 | min |

| | |
|---|---|
| 6) 8° C. | Hold |

40 cycles of steps 2-4 were carried out and the PCR products were run on a gel. The product band (1.9 kb) was excised from the gel and purified using Qiagen's QIAquick Gel Extraction Kit (Qiagen Ltd, Boundary Court, Gatwick Road, Crawley, West Sussex, RH10 9AX, UK) according to the manufacturers instructions and eluted into 30 μl of sterile water (BDH molecular biology grade/filter sterile).

2.2.4 Genomic DNA Cloning and Sequencing

Since the gDNA was amplified using Pfu ultra polymerase which produces blunt ends it was necessary to add ‘A’ overhangs before ligating in to pGEM Teasy. 12.5 μl of purified PCR product was incubated with 12.5 μl 2×PCR Reddy Mix (ABGene) at 70° C. for 30 minutes. The sample was then purified using Qigen Qiaquick gel extraction kit and eluted in 30 μl of molecular biology grade water.

The PCR product was then ligated into pGEM-Teasy (Promega) using the following ligation mixture:

| | |
|---|---|
| 2x Buffer | 5 μl |
| pGEM Teasy | 1 μl |
| PCR product | 3 μl |
| T4 DNA Ligase | 1 μl |

The reaction was incubated over-night at 4° C.

2 μl of the ligation mix were then added to Select 96 cells (Promega) and incubated for 20 min on ice. Cells were then heat shocked at 42° C. for 45 secs and placed back on ice. 250 μl of room temp. SOC medium was then added and the cells incubated for 1 hour at 37° C., with shaking at 220 rpm. 50 and 200 μl amounts were then plated on to LB agar plates containing ampicillin (100 μg/ml), 50 μl X-gal (4%) and 10 μl IPTG (100 mM) and incubated over night at 37° C.

Individual white colonies were picked from each transformation inoculated into LB with ampicillin (100 μg/ml) and incubated over-night at 37° C., with shaking at 220 rpm. Plasmid DNA was extracted using Qiagen miniprep kit according to the manufacturers instructions. 1 μl of plasmid DNA was digested with EcoRI for 1 hour at 37° C. Fragment sizes were calculated to be 3 Kb and 1.6 Kb for gDNA and 3 Kb and 1.2 Kb for cDNA. Clones showing the correct restriction digest pattern were sequenced at MWG Biotech UK Ltd, Waterside House, Peartree Bridge, Milton Keynes, MK6 3BY. The experimentally determined sequence of 2031 OR was identical in the coding regions to that identified by bioinformatic analyses (Example 2).

### Example 3

### cDNA Sequencing and RACE for 2031 OR

The internal sequence of the 2031 OR message was experimentally determined by cloning and sequencing cDNA, and the 5′ and 3′ ends of the gene were determined by RACE (Rapid Amplification of cDNA Ends).

3.1 cDNA Cloning and Sequencing

3.1.1 Preparation of A. fumigatus RNA and cDNA

Fungal cultures were prepared as described in Example 2.2.2. Cultures were harvested by filtration, then washed twice with DEPC-treated water and transferred to a 50 ml Falcon tube. Samples were frozen in liquid nitrogen and stored at −80° C. until required.

To prepare RNA, fungal samples were ground to a fine powder under liquid nitrogen. RNA was then extracted using the Qiagen RNeasy Plant Mini Kit following the protocol for isolation of total RNA from filamentous fungi in the RNeasy Mini Handbook (June 2001, Pages 75-78). The following modifications were used: At step 3, RLC was used as the lysis buffer of choice; At step 7, the Rneasy column was incubated for 5 min at room temperature after addition of RW1; The optional step 9a was carried out; At step 10, 30 μl RNase-free water was added, the samples incubated for 10 min at room temperature, and then centrifuged; At step 11, the elution step was repeated to give a total volume of 60 μl RNA.

DNA contamination was removed from the RNA by the addition of Dnase, using 2 μl DNase per μg RNA, in the presence of 10× DNase buffer and incubating at 37° C. for 2 h. DNase-treated RNA was cleaned up using the RNeasy Plant Mini Kit following the RNeasy Mini Protocol for RNA Cleanup (RNeasy Mini Handbook June 2001, pages 79-81).

To synthesise cDNA from the above RNA the following reaction mixture was prepared: 100 ng-1 μg of DNA-free RNA, 3 μl oligo (dT) (100 ng/μl), and DEPC-treated water to a total volume of 42 μl. Samples were incubated in a heat block at 65° C. for 5 min after which they were allowed to cool slowly to room temperature. Then 2 μl Ultrapure dNTPs, 1 μl reverse transcriptase (Stratascript) and 5 μl 10× reverse transcriptase reaction buffer (Stratascript) were added. Samples were incubated at 42° C. for 1 h, denatured at 90° C. for 5 min and then cooled on ice.

3.1.2 Production of cDNA Constructs

PCR was carried out using the cDNA above to generate cDNA fragments using the primer pair SEQ ID No. 48 (Ox1_for) and SEQ ID No. 49 (Ox3_rev). PCR reactions were carried out using the following reagents and conditions:

PCR Master Mix

| | |
|---|---|
| 10x high fidelity PCR buffer | 5 μl |
| dNTP (clontech: 10 mM) | 1 μl |
| MgSO₄ (50 mM) | 2 μl |
| nH₂O | 37.8 μl |
| Platinum TAQ Polmerase (5 U/μl) | 0.2 μl |
| Forward primer (Ox1_for: 10 pmol/μl stock) | 1 μl |
| Reverse primer (Ox3_rev: 10 pmol/μl stock) | 1 μl |
| cDNA | 2 μl |

PCR Cycle

| | | | |
|---|---|---|---|
| 1) | 94° C. | 5 | min |
| 2) | 94° C. | 30 | sec |
| 3) | 53° C. | 30 | sec |
| 4) | 68° C. | 90 | sec |
| 5) | 68° C. | 10 | min |

| | | |
|---|---|---|
| 6) | 8° C. | Pause |

Cycles 2-4 were run 40 times in total. The amplicon was 1269 bp. The PCR products were purified using Qiagen's QIAquick PCR Purification Kit (Qiagen Ltd, Boundary Court, Gatwick Road, Crawley, West Sussex, RH10 9AX, UK) according to the manufacturers instructions. The purified PCR products were examined on agarose gels.

PCR products were ligated into pGEM-Teasy, used to transform Select 96 cells, and sequenced as described in 2.2.4 above. The cDNA sequence obtained is given as bases 115-1385 of SEQ ID No. 2.

### 3.2 RACE

To determine the 5′ and 3′ ends of the genes, RACE (Rapid Amplification of cDNA Ends) was carried out, using the GeneRacer™ Kit (Invitrogen; cat. No. L1502-01), essentially as per manufacturers instructions.

3.2.1 Preparation of RNA

A. fumigatus biomass was prepared as described in 2.2.2. RNA was prepared using the FastRNA kit (QBIOgene) following the manufacturer's instructions (Revision 6030-999-1J05) with the following amendments: At step 140 mg of biomass was used per extraction; At step 2, samples were processed for 20 seconds at speed 5, incubated on ice for 3 minutes, and processed again for 20 seconds at speed 5; At step 3 samples were centrifuged for 5 minutes; At step 5, 500 μl DIPS were added, mixed, and incubated at room temperature for 2 minutes. Samples were mixed again and incubated for a further 2 minutes; At step 6 two washes in 250 μl SEWS were carried out; At step 7, the pellet was dissolved in 50 μl SAFE buffer.

3.2.2 RACE

1 μg total RNA prepared as described above was de-phosphorylated in a 10 μl reaction using 10 units of calf intestinal phosphate (CIP), 1 μl 10×CIP buffer and 40 U RNaseOut™ (made up to 10 μl in DEPC water) at 50° C. for 1 hour. Samples were then made up to 100 μl with DEPC water and the RNA extracted with 100 μl (25:24:1) phenol:chloroform:isoamyl alcohol. RNA was then precipitated by the addition of 2 μl mussel glycogen (10 mg/ml), 10 μl 3M sodium acetate, pH 5.2 and 220 μl 95% ethanol and the sample frozen on dry ice for 10 minutes. RNA was pelleted by centrifugation at 14,500 rpm for 20 minutes at 4° C., washed with 70% ethanol, air dried and re-suspended in 8 μl DEPC water.

De-phosphorylated RNA (7 μl) was de-capped in a 10 μl reaction with 0.5 U tobacco acid pyrophosphatase (TAP), 1 μl 10×TAP buffer and 40 U RnaseOut™ for 1 hour at 37° C. RNA was extracted with phenol:chloroform and precipitated as above, and then re-suspended in 7 μl DEPC-treated water.

De-phosphorylated, de-capped RNA (7 μl) was added to the pre-aliquoted GeneRacer™ RNA Oligo (0.25 μg) and incubated at 65° C. for 5 minutes. A 10 μl ligation reaction was then set up by the addition of 1 μl 10× ligase buffer, 1 μl 10 mM ATP, 40 U RnaseOut™ and 5 U T4 RNA ligase and incubated at 37° C. for 1 hour. RNA was extracted and precipitated as described previously and re-suspended in 11 μl DEPC-treated water.

First-strand cDNA was prepared by the addition of 1 μl GeneRacer™ Oligo dT primer and 1 μl dNTP mix (10 mM each) to 10 μl ligated RNA and incubated at 65° C. for 5 minutes. The following reagents were added to the 12 μl ligated RNA and primer mix; 4 μl 5× first strand buffer, 2 μl 0.1 M DTT, 1 μl RNaseOut™ and 1 μl SuperScript™ II RT (200 U/μl) and incubated first at 42° C. for 50 minutes and then, to stop the reaction, at 70° C. for 15 minutes. 2 U RNase H was added to the reaction mix and incubated at 37° C. for 20 minutes.

To amplify the 5′cDNA ends a 50 μl PCR reaction was set up using 1 μl of the RACE-ready cDNA prepared above, 1 μl GeneRacer™ 5′ primer, 1 μl reverse gene-specific primer (SEQ ID No. 50; Ox6race_rev: 5 pmol/μl stock), 1 μl dNTP solution (10 mM each), 2 μl 50 mM MgSO₄, 5 μl High Fidelity PCR buffer, 0.5 μl Platinum® Taq DNA Polymerase High Fidelity (5 U/μl) and 38.5 μl sterile water. Cycling parameters are given in Table II below.

A second, nested PCR stage was then set up using 1 μl of the RACE cDNA from the first stage above, 1 μl Nested 5′ primer (supplied with kit), 1 μl reverse gene-specific primer (SEQ ID No. 50; Ox6race_rev: 5 pmol/μl stock), 1 μl dNTP solution (10 mM each), 2 μl 50 mM MgSO₄, 5 μl High Fidelity PCR buffer, 0.5 μl Platinum® Taq DNA Polymerase High Fidelity (5 U/μl) and 38.5 μl sterile water. Cycling parameters are given in Table II below.

To amplify 3′ ends a 50 μl PCR reaction was set up using 1 μl of the RACE-ready cDNA prepared above, 1 μl GeneRacer™ 3′ primer (10 μM), 1 μl forward gene-specific primer (SEQ ID No. 51; Ox7race_for: 5 μmol/μl stock), 1 μl dNTP solution (10 mM each), 2 μl 50 mM MgSO₄, 5 μl High Fidelity PCR buffer, 0.5 μl Platinum® Taq DNA Polymerase High Fidelity (5 U/μl) and 38.5 μl sterile water. Cycling parameters are given in Table II below:

A second, nested PCR stage was then set up using 1 μl of the 3′ RACE cDNA from the first stage above, 1 μl Nested 3′ primer (supplied with kit), 1 μl reverse gene-specific primer (SEQ ID No. 52; Ox8race_for: 5 pmol/μl stock), 1 μl dNTP solution (10 mM each), 2 μl 50 mM MgSO₄, 5 μl High Fidelity PCR buffer, 0.5 μl Platinum® Taq DNA Polymerase High Fidelity (5 U/μl) and 38.5 μl sterile water. Cycling parameters are given in Table II below.

TABLE II
Cycling parameters for 5′ and 3′ RACE

| 5′ and 3′ RACE | | | | | Nested PCR | | | | |
|---|---|---|---|---|---|---|---|---|---|
| 94° C. | 2 | min | 1 | cycle | 94° C. | 2 | min | 1 | cycle |
| 94° C. | 30 | s | 5 | cycles | 94° C. | 30 | sec | 25 | cycles |
| 72° C. | 1 | min | | | 67° C. | 30 | sec | | |
| 94° C. | 30 | s | 5 | cycles | 68° C. | 1 | min | | |
| 70° C. | 1 | min | | | 68° C. | 10 | min | 1 | cycle |

| | | | | | | |
|---|---|---|---|---|---|---|
| 94° C. | 30 | s | 25 | cycles | 8° C. | Hold |
| 64° C. | 30 | s | | | | |
| 68° C. | 1 | min | | | | |
| 68° C. | 10 | min | 1 | cycle | | |

| | |
|---|---|
| 8° C. | Hold |

5′ and 3′ RACE confirmed the predicted 5′ ATG and 3′ stop codon as well as giving the 5′ and 3′ untranslated regions shown as bases 1-114 and 1385-1921 of SEQ ID No. 2. The coding sequence for 2031 OR thus determined was identical to that given as bases 299-469 and 520-1618 of the gDNA gien as SEQ ID No. 1.

### Example 4

### Identification of Other Fungal 2031 ORs and Related Genes

Homologs of A. fumigatus 2031 OR were identified in other fungi and bacteria by means of bioinformatics analysis. Sequences identified by bioinformatics can be used to design primers which in turn can be used in PCR to generate DNA coding for the 2031 OR homolog.

Alternatively, degenerate PCR can be used to obtain sequence for novel genes, which can then be used to generate probes for screening cDNA or genomic libraries of the organism of interest to identify clones containing the 2031 OR homolog. As a further alternative, Southern blots using fragments of genes from one species as probes can be used to identify the presence of a homolog in the genome of a second species. The same probe can then by used to screen cDNA or genomic DNA libraries. Once clones corresponding to the novel genes have been identified they can be expressed for functional characterisation of the protein.

### 4.1 Identification of Homologs by Bioinformatics

Analysis of the 2031 OR protein sequence with PFAM identified this as a member of the Oxidored FMN family (PF00724), E-value 3.6e-57. This includes the well-characterised “Old Yellow Enzyme” proteins of S. cerevisiae and other fungi.

Homologs of A. fumigatus 2031 OR sequence were identified by database searches (see Table III). Where necessary, matching contigs were down-loaded and genes predicted from genomic DNA by Genscan analysis, blast searches, alignment and visualisation with Artemis as described in Example 2. Protein and nucleotide multiple alignments were generated for 2031 OR and related genes (FIGS. 1 and 2).

Protein and nucleic acid multiple alignments are generated by means of programs such as ClustalX (Thompson et al., 1994, Nucleic Acids Research, 22, 4673-4680; Thompson et al., 1997, Nucleic Acids Research, 24, 4876-4882;) and/or using manual alignment editors such as Align (Hepperle, D., 2001: Multicolor Sequence Alignment Editor. Institute of Freshwater Ecology and Inland Fisheries, 16775 Stechlin, Germany).

TABLE III
2031 homologs identified by database searches

| Contig/EST/ | | | | | |
|---|---|---|---|---|---|
| predicted | E- | SEQ ID No. | | | Species (details of search |
| gene | value¹ | EST/gDNA | CDNA² | Protein | given in footnotes) |
| 4929 | 6.6e−81 | 4 | 5 | 6 | Aspergillus fumigatus³ |
| 4951 | 1.1e−68 | 7 | — | 8 | Aspergillus fumigatus³ |
| 4875 | 5.7e−13 | — | — | — | Aspergillus fumigatus³ |
| 4961 | 3.2e−10 | — | — | — | Aspergillus fumigatus³ |
| 1.112 | 3e−33 | 9 | — | 10 | Aspergillus nidulans⁴ |
| 6-2431 | 2.6e−77 | 11 | — | 12 | Candida albicans⁵ |
| 6-2464 | 5.9e−50 | 13 | — | 14 | Candida albicans⁵ |
| 6-2460 | 5.8e−19 | — | — | — | Candida albicans⁵ |
| A36990 | 1e−15 | — | — | — | Candida albicans⁶ |
| NCU07452.1 | 7e−94 | 15 | — | 16 | Neurospora crassa⁷ |
| NCU08900.1 | 2e−19 | 17 | 18 | 19 | Neurospora crassa⁷ |
| NCU04452.1 | 2e−23 | — | — | — | Neurospora crassa⁷ |
| MG04569.3 | 1e−106 | 20 | 21 | 22 | Magnaporthe grisea⁸ |
| MG03823.3 | 8e−19 | 43 | — | 44 | Magnaporthe grisea⁸ |
| NP_595868 | 1e−05 | 23 | — | 24 | Schizosaccharomyces |
| | | | | | pombe⁶ |
| OYE1 | 1e−15 | — | — | — | Saccharomyces cerevisiae⁶ |
| OYE2 | 4.5e−19 | — | — | — | Saccharomyces cerevisiae⁹ |
| OYE3 | 1.0e−16 | — | — | — | Saccharomyces cerevisiae⁹ |
| FsCon[0063] | 1e−82 | 28 | 29 | 30 | Fusarium |
| (EST contig) | | | | | sporotrichioides¹⁰ |
| Gz15771741 | 5e−76 | 36 | 37 | 38 | Fusarium graminearum^{10 0} |
| Mg[0281] | 2e−67 | 39 | | 40 | Mycosphaerella |
| (EST contig) | | | | | graminicola¹⁰ |
| CtCon[0249] | 1e−55 | 25 | 26 | 27 | Colletotrichium trifolii¹⁰ |
| (EST contig) | | | | | |
| FsCon[0458] | 1e−42 | 34 | | 35 | Fusarium |
| (EST contig) | | | | | sporotrichioides¹⁰ |
| FsCon[0237] | 1e−40 | 31 | 32 | 33 | Fusarium |
| (EST contig) | | | | | sporotrichioides¹⁰ |
| Mga0328f | 3e−35 | 41 | | 42 | Mycosphaerella |
| | | | | | graminicola¹⁰ |
| T44612 | 1e−52 | — | — | — | Pseudomonas putida¹¹ |
| NP_625402 | 1e−79 | — | — | — | Streptomyces coelicolor¹¹ |
| NP_295913 | 1e−78 | — | — | — | Deinococcus radiodurans¹¹ |
| AF320254 | 5e−55 | — | — | — | Deinococcus radiodurans¹¹ |
| FG00074.1 | | 82 | 82 | 83 | Fusarium graminearum¹² |
| Contig 1.2 | 1e−71 | 84 | 84 | 85 | Ustilago maydis¹³ |

¹E-values for blast scores refer to searches with 2031 OR protein unlesss pecified otherwise in footnotes.
²A cDNA was generated in cases where either the gene contains multiple exons, or there are probable frame-shift errors from sequencing of the EST, or the EST given is the non-coding strand.
³Search of the A. fumigatus genome at TIGR (tblastn) with NP_595868.
⁴Search of A. nidulans genome held on local machine (tblastn).
⁵Search of the C. albicans genome at sequence.stanford (blastp).
⁶Search of the non-redundant protein sequence database (nr) at blast.genome (blastp).
⁷Search of the N. crassa predicted proteins at broad.mit. (blastp).
⁸Search of the M. grisea predicted proteins at boad.mit. (blastp).
⁹Search of S. cerevisiae orf proteins.
¹⁰Search of COGEME pathogenic fungal EST database at cogeme (tblastn, max E-val = 0.1).
¹¹Search of NCBI non-redundant protein database on local machine with SEQ ID No. 1 (blastx). Only a selected set of hits against bacterial proteins are shown.
¹²Search of F. graminearum predicted proteins held on local machine (blastp).
¹³Search of U. maydis contigs held on local machine (tblastn).

To clarify the relationships between the 2031 OR, OYE and the hits identified from blast searches, phylogenetic analysis was carried out. The PHYLIP suite of programs was used (Felsenstein, Felsenstein, J., 2002. PHYLIP (Phylogeny Inference Package) version 3.6a3. Distributed by the author. Department of Genome Sciences, University of Washington, Seattle). The multiple alignment used for the analyses was essentially that given in FIG. 1 with partial sequences, gapped regions and unreliably aligned sections excluded. A distance matrix was generated using PROTDIST with the Jones-Taylor-Thornton model and the tree inferred using FITCH with global rearrangements and 10 jumbles of input order. 100 bootstrap replicates were generated using SEQBOOT, distance matrices generated using PROTDIST as above, trees inferred using NEIGHBOUR, and then bootstrap values and the consensus tree were calculated using CONSENSE. Trees were viewed using TREEVIEW (Page, 1996 Page, R. D. M., 1996. TREEVIEW: An application to display phylogenetic trees on personal computers. Computer Applications in the Biosciences 12, 357-358.)

Phylogenetic analysis identified a clade supported by good bootstrap values, which included A. fumigatus 2031 OR and other enzymes. This could be distinguished from a clade containing OYE enzymes which was also supported by good bootstrap values. Bacterial homologs of both 2031 OR and OYE (not shown) were also identified. We have therefore identified a set of 2031 OR homologs which, surprisingly, is distinct from the well-characterised OYE family, and which, by virtue of the essentiality demonstrated for A. fumigatus 2031 OR, represents a set of potential targets for anti-fungal drugs

### 4.2 Identification of Homologs by Degenerate PCR

4.2.1. Preparation of Genomic DNA from Organism of Interest

Fungal cultures are prepared using methods suitable for particular species. For example, Aspergillus and Candida species, Cryptococcus neoformans, Fusarium solani and Trichophyton species are maintained on Sabouraud dextrose agar at 30-35° C.; Leptosphaeria nodorum on Malt agar medium (30 g/L malt extract; 15 g/L Bacto-agar, pH 5.5), 24.0° C.; Magnaporthe grisea on Oatmeal agar (6.7 g/L agar, 53.3 g/L instant oatmeal) 25.0° C., or Cornmeal agar (Difco 0386), 26.0 C; Phytophthora capsici cultures were maintained on V-8 agar at 24° C.; Pyricularia oryzae cultures were maintained on rice polish agar at 24° C. under white fluorescent lights (12 hr artificial day), and were subcultured every 7-14 days by the transfer of mycelial plugs to fresh plates; Pythium ultimum cultures were maintained on PDA at 24° C., and subcultured every 7 days by the transfer of aerial mycelium to fresh plates with an inoculating needle; Rhizoctonia solani cultures were maintained on PDA at 24° C. under fluorescent lights (12 h artificial day), and subcultured every 7 days by the transfer of mycelial plugs to fresh plates; Ustilago maydis cultures were maintained on PDY agar at 30° C. in the dark, and subcultured by re-streaking.

Genomic DNA was prepared from cultures using standard methodologies, e.g. using the Qiagen DNeasy Plant Kit, or using methods described in Example 2.2.

4.2.2 PCR

Primers (SEQ ID Nos. 53 and 54) were designed on the 2031 OR-specific regions given as regions 2 and 6 in FIG. 2. However, those skilled in the art will appreciate that it may be necessary to try alternative primers. PCR reactions using the above primer pair are set up as follows:

12.5 μl 2× ReddyMix PCR mastermix (ABIgene)
1 μl primer SEQ ID No. 53 (5 pmol)
1 μl primer SEQ ID No. 54 (5 pmol)
template gDNA (1.5-4 μg/ml)
nuclease-free water to give a final volume of 25 μl

The reactions are run using the following conditions on a Biometra personal PCR cycler (Thistle Scientific Ltd, DFDS House, Goldie Road, Uddington, Glasgow, G71 6NZ):—

| | | |
|---|---|---|
| Step1 | 95° C. | 5 min |
| Step2 | 95° C. | 1 min |
| Step3 | 53° C. | 1 min 30 sec |
| Step4 | 68° C. | 2 min 30 sec |
| Step5 | 72° C. | 10 min |
| Step6 | 4° C. | Hold |

30 cycles of steps 2-4 are carried out. The PCR products are purified (to remove residual enzymes and nucleotides) using Qiagen's QIAquick PCR Purification Kit (Qiagen Ltd, Boundary Court, Gatwick Road, Crawley, West Sussex, RH10 9AX, UK) according to the manufacturers instructions and eluted into 40 μl of sterile water (BDH molecular biology grade/filter sterile). The purified PCR products are examined on 1% agarose gels.

Those skilled in the art will appreciate that degenerate PCR may require variations in a number of parameters in the attempts to generate a product. These include primer concentration, template concentration, concentration of Mg²⁺ ions, elongation and annealing times, and annealing temperature. Variations in temperature can be accommodated by the use of a gradient PCR machine.

The purified PCR products are cloned into pPEM-Teasy (Promega) and then transformed into XL10-Gold® Kan ultracompetent E. coli cells according to the manufacturer's instructions. The transformation reactions are then plated onto LB agar plates containing ampicillin (100 μg/ml), 50 μl X-gal (4%) and 10 μl IPTG (100 mM). Following overnight incubation at 37° C., individual white colonies from each transformation are sub-cultured into LB broth containing ampicillin (100 μg/ml). After overnight incubation at 37° C. with shaking, plasmids are extracted using Qiagen spin mini plasmid extraction kits according to the manufacturers instructions and sent away for full-length sequencing.

### 4.3 Identification of Homologs by Southern Blotting

4.3.1 Digestion of Genomic DNA and Transfer to Nylon Membranes

Genomic DNA from the fungi of interest are digested with the appropriate restriction enzyme and run on 0.8% agarose gel. The gel is then submerged in 250 mM HCl for no more than 10 mins, with shaking, at room temperature, after which the gel is rinsed with sterilised RO water.

Transfer of the DNA onto nylon membrane is carried out using 0.4 M NaOH. Transfer protocols and apparatus are well known and are described in e.g. Sambrook et al., (1989), Molecular Cloning, 2^{nd} Edition, Cold Spring Harbor Laboratory Press. After transfer, the DNA is fixed to the membrane by baking at 120° C. for 30 min. The membrane can then be used immediately, or stored dry for future use.

4.3.2. Preparation of Probe

Probes are generated either by restriction digests of DNA or by PCR of an appropriate region. A suitable probe can be generated by PCR using the primer pair SEQ ID Nos. 53 and 54, A. fumigatus genomic DNA, and the methods give in 4.2.2.

1 μg DNA template is diluted in molecular biology water to a total volume of 16 μl, denatured in a boiling water bath for 10 mins, and quickly chilled on ice. 4 μl DIG-High Prime (1 mM dATP, 1 mM dCTP, 1 mM dGTP, 0.65 mM dTTP, 0.35 mM alkali-labile-digoxygenin-11-dUTP, 1 U/μl labelling grade Klenow enzyme, 5× reaction buffer, in 50% (v/v) glycerol) is then added and the reaction incubated at 37° C. for 20 hours, after which 2 μl of 200 mM EDTA pH 8.0 is added to terminate the labelling reaction. The labelling efficiency is estimated by comparison with DIG-labelled control DNA.

4.3.3. Prehybridisation and Hybridisation

The membrane is placed in a hybridisation tube containing 20 ml of prehybridisation solution (DIG Easy Hyb, Roche) per 100 cm² of membrane surface area and prehybridised at 42° C. for 2 hours in a hybridisation oven. The DIG-labelled probe is denatured by heating in a boiling water bath for 10 min and then chilled directly on ice. The probe is then diluted to 200 ng/mL in hybridisation solution (Easy Hyb, Roche; at least 5 mL of hybridisation solution is required per hybridisation). The prehybridisation solution is discarded from the hybridization tube and the hybridisation solution containing the DIG-labelled probe added quickly. The hybridisation then proceeds overnight at a 42° C. in the hybridisation oven. The optimum temperature is dependant on probe size and homology with target sequence and was determined empirically.

After hybridisation, the membrane is washed twice at 42° C., 5 mins per wash, with 50 mL of stringency wash solution (3×SSC, 0.1% SDS; where 20×SSC buffer is 3 M NaCL, 300 mM sodium citrate, pH 7.0), followed by two washes at RT, 15 min per wash, in 50 mL stringency wash solution. The stringency of these washes can be decreased by increasing the SSC concentration to 6×SSC, 0.1% SDS and/or decreasing the wash temperatures.

### 4.3.4. Detection

The membrane is washed in 20 mL washing buffer (100 mM Maleic acid, 150 mM NaCl; pH 7.5; 0.3% v/v Tween 20), and then incubated successively with the following; 20 mL blocking solution (1% w/v blocking reagent for nucleic acid hybridisation, Roche, dissolved in 100 mM maleic acid, 150 mM NaCl, pH 7), for 30 min at room temperature; Anti-DIG-alkaline phosphatase (Roche) diluted 1:5,000 in blocking buffer, 30 min at room temperature; Washing buffer, two washes each of 15 min at room temperature; Detection buffer (100 mM Tris-Hcl, 100 mM NaCl; pH 9.5), 2 min at room temperature. The membrane is then removed, placed on top of an acetate sheet, and ˜0.5 ml (per 100 cm²) of CSPD or CDP-star added to the top of the membrane. A second sheet of acetate is then placed over the surface of the membrane, the assembly incubated for 5 min at room temperature and then sealed in a plastic bag. The assembly is then exposed to X-ray film for between 15 min and 1 hour. Optimal exposure time is determined empirically by increasing exposure time up to 24 hours.

The presence of a band on the gel is evidence of a gene in the genomic DNA of interest. The molecular weight of the band depends on the size of the restriction fragment that contains the gene.

### Example 5

### Expression During Infection of Wax Moth Larvae (Galleria melonella) and Mice with A. fumigatus

5.1 Preparation of cDNA from Infected Wax-Moth Larvae

Wax moth larvae have been shown to be good model systems in which to study Candida infection (Cotter et al., 2000, FEMS Immunol Med Microbiol 27, 163-9; Brennan et al., 2002, FEMS Immunol Med Microbiol 34, 153-7). We have found that this insect system is also a good system in which to study Aspergillus infection (D. Law and J. Rooke, manuscript in preparation).

5.1.1 Growth and Infection of Wax-Moth Larvae

Spores of A. fumigatus (AF293), grown on Sabaraud Dextrose agar, were harvested and re-suspended in PBS/Tween 80. Spores were washed and the concentration adjusted such that a 10 μl inoculum will cause death in 90% of the test group 3-4 days after infection (for AF293 this is 5.0-7.0×10⁸ cfu/ml). Inoculum concentration was estimated using an improved Neubauer haemocytometer counting chamber and confirmed by TVC enumeration.

Wax moth larvae were purchased from Livefood UK, Somerset, UK (www.livefood.co.uk), and were maintained in the dark at room temperature in wood shavings prior to infection. Healthy larvae (250 mg+/−50 mg) were selected and incubated at 4° C. for 10 minutes immediately prior to infection to immobilise them. Larvae were then injected through the cuticle of the left last pro-leg with 10 μl spore suspension (100× stock), using a sterile Hamilton syringe. Larvae were then transferred to a sterile Petri dish. The following controls were also established: Larvae injected with 10 μl PBS/Tween only; larvae injected with 10 μl heat killed spores (killed by incubation for 20 min 100° C.); larvae pierced but not injected; and untouched larvae. Larvae were incubated at 30° C. and monitored at least twice daily. All treatments and controls were carried out on batches of 10 larvae. Larval deaths and general health condition was recorded every 24 hrs and dead or moribund larvae were removed from the test group.

5.1.2 Preparation of DNA-Free RNA from Aspergillus fumigatus-Infected Wax Moth Larvae (Galleria melonella).

cDNA was prepared from the following sources: Uninfected larvae; larvae after 48 h infection with A. fumigatus (early infection); larvae after 72 h infection with A. fumigatus (late infection); larvae infected with heat-killed A. fumigatus spores; and A. fumigatus grown in Sabaraud Dextrose agar broth for 16 hr.

Frozen larvae were ground to a fine powder under liquid nitrogen in a mortar and pestle previously baked at 22° C. overnight, treated with RNaseZAP, rinsed with DEPC-treated water (0.1% (v/v) DEPC, stirred for 1 h and autoclaved for 1 h) and cooled with liquid nitrogen. Ground sample was transferred to Eppendorf tubes (no more than 50 mg per tube) and total RNA extracted using the Qiagen RNeasy Plant Mini Kit following the protocol for isolation of total RNA from filamentous fungi in the RNeasy Mini Handbook (June 2001, Pages 75-78).

The following modifications were used: At step 3, 600 μl RLT was added to each 50 mg tissue and vortexed; At step 4, samples were centrifuged for 3 min at maximum speed; At step 6, all samples from the same tissues were applied to the same RNeasy column; At step 7, RNeasy column was incubated for 5 min at room temperature after addition of RW1; Optional step 9a was carried out twice; At step 10, 30 μl RNase-free water was added, samples incubated for 10 min at room temperature, and then centrifuged for 1 min at 14,000 RPM; At step 11, the elution step was repeated to give a total volume of 60 μl RNA. A sample of the RNA was run on a 1.5% agarose gel and the amount of RNA quantified using the molecular marker. RNA was then stored at −80° C.

A portion of the RNA was Dnase treated using 2 μl RNase-free DNase (Promega) per μg RNA, in the presence of 10× DNase buffer (Promega) at 37° C. for 4 h. The RNA was then cleaned up using the Qiagen RNeasy Plant Mini Kit following the RNeasy Mini Protocol for RNA Cleanup (RNeasy Mini Handbook June 2001, pages 79-81), but including a further DNase treatment step during clean-up as in the Rneasy handbook.

The following modifications were made: Optional step 5a was carried out; At step 6, 30 μl RNase-free water was added, samples incubated for 10 min at room temperature and then centrifuged for 1 min at 14,000 RPM; At step 7, the eluate from step 6 was transferred onto the RNeasy column, incubated for 10 min at room temperature, and then centrifuged for 1 min at 14,000 RPM. A sample of the DNase-treated RNA was run on an agarose gel, quantified and stored at −80° C.

5.1.3 Checking RNA Samples for DNA Contamination

To verify the absence of genomic DNA from the RNA samples, PCR was carried out using primers that amplify the β-tubulin gene (SEQ ID Nos. 77 and 78). In the absence of a reverse-transcription step, only gDNA will be detected and thus any gDNA contamination will be revealed. The following reaction mixture was set up:

12.5 μl 2× ReddyMix PCR mastermix (ABIgene)
1 μl each primer (5 pmol)
template gDNA (1.5-4 μg/ml)
nuclease-free water to give a final volume of 25 μl

The reactions were run using the following conditions on a Biometra personal PCR cycler (Thistle Scientific Ltd, DFDS House, Goldie Road, Uddington, Glasgow, G71 6NZ):—

| | | |
|---|---|---|
| Step1 | 95° C. | 5 min |
| Step2 | 90° C. | 1 min |
| Step3 | 51° C. | 1 min |
| Step4 | 68° C. | 1 min |
| Step5 | 68° C. | 10 min |
| Step6 | 4° C. | Hold |

40 cycles steps 2-4

If a PCR product was observed, genomic DNA was present and the sample was DNase-treated again. If the PCR was negative, no DNA was present in the sample.

5.1.4 Preparation of cDNA

300 μg DNA-free RNA and 3 μl oligo (dT) (100 ng/μl) were added to an RNase-free 0.5 ml microcentrifuge tube, and made up a total volume of 42 μl with DEPC-treated water. Samples were mixed and incubated in a heat block at 65° C. for 5 min and then slowly cooled to room temperature. 2 μl Ultrapure dNTPs (10 mM each, Clontech), 1 μl stratascript reverse transcriptase (Stratagene) and 5 μl 10× reverse transcriptase reaction buffer were then added. The samples were incubated at 42° C. for 1 h, denatured at 90° C. for 5 min and then cooled on ice. Samples were dispensed in 5-10 μl aliquots and stored at −20° C.

5.2. Preparation of cDNA from Infected Mice

5.1.1 Infection of Mice with A. fumigatus and Extraction of Tissues.

Mice were infected with Aspergillus fumigatus and organs harvested as follows. Thirteen male CD1 mice were injected with the immunosuppressant cyclophosphamide (0.025 g/ml; 200 mg/kg) IV via the tail vein. After 72 hours, twelve mice were injected with 0.15 ml Aspergillus fumigatus AF293 conidia (7.5×10⁵/ml). 11 hours after infection, four mice were sacrificed with an overdose of inhaled halothane. The brain, lungs, liver and kidney were removed, frozen by immersion in liquid nitrogen, and stored at −70° C. A further four mice were also sacrificed at 24 and 48 hours after infection.

RNA was prepared from mouse tissues as described for wax moth larvae above (5.1.2 and 5.1.3).

5.2.2 Preparation of cDNA from DNA-Free RNA.

cDNA was prepared from DNA-free RNA using the Promega Reverse Transcription kit, following the protocol as supplied with the product (Technical Bulletin No. 099). In a modification to the protocol, the cDNA synthesis reaction was incubated for 60 min at 42° C. rather than for the suggested 15 min. Samples were stored in 5-10 μl aliquots at −20° C.

### 5.3 Design and Optimisation of Primers

Primers were designed against the 2031 OR cDNA sequence using Beacon Designer 2.1 (Premier Biosoft) with the following parameters; Target Tm=58±8° C.; Length of primers=16-24; Amplicon length=75-150 bp. All other settings were default. Care was taken to choose primers that would not form dimers or other secondary structures. Secondary structures of amplicons were calculated using mfold and primer sets giving an amplicon with little or no secondary structure were chosen. The resulting primers are given as SEQ ID Nos. 79 and 80.

To determine optimum annealing temp for the primer set, a gradient PCR was run on an Icycler PCR machine (Biorad), using A. fumigatus AF293 genomic DNA as a template and the following reaction mixture:

### 112.5 μl Abgene PCR Reddymix

9 μl SEQ ID No. 79; OXRED 2031F6 (5 pm/μl)
9 μl SEQ ID No. 80; OXRED 2031R5 (5 pm/μl)

### 85.5 μl H₂O

9 μl AF293 gDNA (10 ng/ul)

For the negative control, the gDNA was omitted and the amount of water increased correspondingly.

For each mix, 25 μl was pipetted into 8 wells on a multiwell plate, and each well run at a different temp (between 50 and 65° C.) with the following conditions:

### Step 1. 95° C.—5 min

### Step 2. 95° C.—1 min

### Step 3. Gradient 50-65° C.—1.5 min

### Step 4. 72° C.—1 min

### Step 5. 72° C.—10 min

Step 6. 8° C.—hold
Steps 2-4 were run for 30 cycles

The PCR products were run on a 2% agarose gel. A single band of the correct size of 148 bp was seen on the gel for all the temperatures, and the optimum was found to be 63° C.

### 5.4 Testing Species—Specificity of Primers

The real-time primers designed above were further tested to ensure that mouse nucleic acid was not amplified using these primers. Four reactions were set up, each containing the following:

### 12.5 μl Abgene Reddymix

1 μl primer SEQ ID No. 79
1 μl primer SEQ ID No. 80

### 9.5 μl H2O

and either; 1 μl infected mouse kidney cDNA (50 ngμl; experimental); 1 μl uninfected mouse kidney cDNA (50 ng/μl; uninfected control); 1 μl AF293 gDNA (10 ng/μl; positive control); 1 μl water (negative control).

The following PCR settings were used:

### Step 1 95° C.—5 min

### Step 2 95° C.—1 min

### Step 3 63° C.—1.5 min

### Step 4 72° C.—1 min

### Step 5 72° C.—10 min

Step 6 8° C.—hold
Steps 2-4 were run 40 times

The PCR products were run on a 2% agarose gel. A. fumigatus genomic DNA gave a band of 148 bp, the expected size, but no bands were seen in uninfected or infected mouse cDNA. These primers therefore appeared to be specific.

### 5.5 Real-Time PCR to Detect Expression in Infected Larvae

PCR reactions were set up using the Biorad iQ SYBR green supermix as follows:

### 14 μl Primer SEQ ID No. 79

### 14 μl Primer SEQ ID No. 80

### 175 μl SYBR mix

### 133 μl H₂O

Four reactions were set up containing 72 μl of the above mix and either; 3 μl H₂O; 3 μl uninfected larvae cDNA (50 ng/μl); 3 μl AF293 gDNA (5 ng/μl); or 3 μl infected larvae cDNA (50 ng/μl) were added. 3×25 μl aliquots of each reaction were aliquoted into an Abgene multiwell plate, the plate sealed with optical sealing tape (Biorad), then placed in a Biorad Icycler real-time PCR machine. Reactions were run with the following conditions:

| | | | |
|---|---|---|---|
| Step1. | 95.0° C. | 3 | min |
| Step2. | 95.0° C. | 30 | sec |
| Step3. | 63.0° C. | 30 | sec |

Data collection and real-time analysis enabled.

| | | | |
|---|---|---|---|
| Step4. | 72.0° C. | 15 | sec |

60 cycles of steps 2-4.

| | | | |
|---|---|---|---|
| Step5. | 95.0° C. | 30 | sec |
| Step6. | 50.0° C. | 30 | sec |
| Step7. | 50.0° C. | 10 | sec |

90 cycles of step 7 with setpoint temperature increased by 0.5° C. after each cycle starting with cycle 2. Melt curve data collection and analysis enabled.

Results are shown in Tables IV and V. Expression of 2031 OR was demonstrated in both Af293 cDNA (Ct=25.8) and in infected larvae (Ct=32.3). Therefore, the message is expressed both in A. fumigatus cultures and in A. fumigatus from infected larvae. The negative and uninfected larvae controls give only primer dimers and non-specific products.

TABLE IV
PCR Quantification Spreadsheet Data for SYBR-490

| Well | Identifier | Ct |
|---|---|---|
| C08 | infected larvae (50 ng) | 33 |
| C09 | infected larvae (50 ng) | 32.4 |
| C10 | infected larvae (50 ng) | 31.4 |
| D03 | Negative | 51.3 |
| D04 | Negative | N/A |
| D05 | Negative | 55.6 |
| H03 | uninfected larvae | 36.4 |
| H04 | uninfected larvae | N/A |
| H05 | uninfected larvae | N/A |
| H08 | A. fumigatus gDNA (5 ng) | 25.8 |
| H09 | A. fumigatus gDNA (5 ng) | 26 |
| H10 | A. fumigatus gDNA (5 ng) | 25.8 |

Data Analysis Parameters: Calculated threshold was replaced by the user selected threshold 7.4. User selected baseline cycles were 2 to 10.

TABLE V
Melt Curve Analysis Spreadsheet Data for SYBR-490

| Well | Well Identifier | Peak ID | Melt Temp |
|---|---|---|---|
| C8 | infected larvae (50 ng) | C8.1 | 88.5 |
| C9 | infected larvae (50 ng) | C9.1 | 88.5 |
| C10 | infected larvae (50 ng) | C10.1 | 88.5 |
| D3 | Negative | D3.1 | 78 |
| D5 | Negative | D5.1 | 81.5 |
| | | D5.2 | 77.5 |
| H3 | uninfected larvae | H3.1 | 81.0 |
| H5 | uninfected larvae | H5.1 | 78.0 |
| H8 | A. fumigatus gDNA (5 ng) | H8.1 | 89.0 |
| H9 | A. fumigatus gDNA (5 ng) | H9.1 | 89.0 |
| H10 | A. fumigatus gDNA (5 ng) | H10.1 | 89.0 |

Melt Curve Analysis Parameters; Threshold for automatic peak detection was set at 2.64.

5.6 Real-Time PCR to Detect Expression in Infected Mouse Kidney cDNA.

Real-time experiments similar to those described in 5.5 using 1 μl of infected mouse cDNA showed no amplification (data not shown). The experiment was therefore carried out using an increased amount of infected mouse cDNA with the following conditions:

### 18 μl Primer SEQ ID No. 79

### 18 μl Primer SEQ ID No. 80

### 225 μl SYBR mix

### 99 μl H₂O

Four reactions were set up containing 60 μl of the above mix and either; 15 μl H₂O; 3 μl uninfected mouse kidney (50 ng/μl)+12 μl H₂O; 15 μl infected mouse kidney—48 h post-infection (50 ng/μl); or 3 μl AF293 cDNA (5 ng/μl)+12 μl H₂O were added. 3×25 μl aliquots of each reaction were aliquoted into an Abgene multiwell plate, the plate sealed with optical sealing tape (Biorad), then placed in a Biorad Icycler real-time PCR machine. Reactions were run with the following conditions:

| | | | |
|---|---|---|---|
| Step1. | 95.0° C. | 3 | min |
| Step2. | 95.0° C. | for 30 | sec |
| Step3. | 63.0° C. | for 30 | sec |

Data collection and real-time analysis enabled.

| | | | |
|---|---|---|---|
| Step4. | 72.0° C. | for 15 | sec |

60 cycles of steps 2-4.

| | | | |
|---|---|---|---|
| Step5. | 95.0° C. | for 30 | sec |
| Step6. | 50.0° C. | for 30 | sec |
| Step7. | 50.0° C. | for 10 | sec. |

90 cycles of step 7 with setpoint temperature increased by 0.5° C. after
each cycle starting with cycle 2. Melt curve data collection and analysis
enabled

Expression of A. fumigatus AF293 2031 OR was seen in cDNA (Ct=28.8) but only in 2 of the 3 infected mouse kidney reactions (Ct values=34.4, 41.2) (Tables VI and VII). The product in the other infected kidney cDNA reaction (well A12) was a primer dimer or a non-specific product (Tm=81° C. on the melt curve), whereas the correct 2031 OR product has a Tm of 88.5° C. (Tables VI and VII). The negative and uninfected kidney controls gave only primer dimers and non-specific products.

TABLE VI
PCR Quantification Data for SYBR-490

| Well | Identifier | Ct |
|---|---|---|
| A10 | infected kidney (250 ng) | 34.4 |
| A11 | infected kidney (250 ng) | 41.2 |
| A12 | infected kidney (250 ng) | 38 |
| D02 | Negative | 50.3 |
| D03 | Negative | 54.6 |
| D04 | Negative | 46.2 |
| H02 | uninfected kidney | 52.8 |
| H03 | uninfected kidney | 54 |
| H04 | uninfected kidney | 51.8 |
| H10 | AF293 (5 ng) | 28.7 |
| H11 | AF293 (5 ng) | 28.7 |
| H12 | AF293 (5 ng) | 30 |

Calculated threshold was replaced by the user selected threshold 5.4. User selected baseline cycles were 2 to 10.

TABLE VII
Melt Curve Analysis Spreadsheet Data for SYBR-490

| Well | Well Identifier | Peak ID | Melt Temp |
|---|---|---|---|
| A10 | infected kidney (250 ng) | A10.1 | 88.5 |
| A11 | infected kidney (250 ng) | A11.1 | 88.5 |
| A12 | infected kidney (250 ng) | A12.1 | 81.0 |
| D2 | Negative | D2.1 | 79.0 |
| D3 | Negative | D3.1 | 78.0 |
| D4 | Negative | D4.1 | 78.0 |
| H2 | uninfected kidney | H2.1 | 78.5 |
| H3 | uninfected kidney | H3.1 | 77.5 |
| H4 | uninfected kidney | H4.1 | 90.5 |
| H10 | AF293 (5 ng) | H10.1 | 88.5 |
| H11 | AF293 (5 ng) | H11.1 | 88.5 |
| H12 | AF293 (5 ng) | H12.1 | 88.5 |

Threshold for automatic peak detection was set at 2.09.

A. fumigatus 2031 OR is therefore clearly expressed during infection of wax moth larvae. 2031 OR is only expressed at a very low level during infection of mouse kidney, since increased amounts of template had to be used to give a signal. The expression during infection suggests that the gene product may be a suitable target for an anti-fungal drug.

### Example 6

### Expression of Recombinant 2031 OR and/or Fragments

Recombinant proteins or fragments were expressed to enable detailed study of function and as the starting point for the development of a high-throughput screen for inhibitory compounds.

6.1 Production of cDNA Constructs

PCR was carried out using cDNA prepared as described above to generate polynucleotides encoding 2031 OR sequence essentially corresponding to SEQ ID No. 3. PCR reactions were carried out using the following reaction mixture and conditions. All Reagents were present in the KOD kit (Novagen).

### 2.5 μl 10×PCR Buffer

5 μl dNTPs (2 mM)

### 2 μl MgSO₄ (25 mM)

1 μl primer A (5 pmol) (SEQ ID No. 55; SL_OxXa30F5)
1 μl primer B (5 pmol) (SEQ ID No. 56; SL-OxXa30R7)
1 μl template cDNA
11.5 μl nuclease-free water

### 1 μl KOD Polymerase

PCR reactions were run using the following conditions:—

| | | |
|---|---|---|
| Step1 | 94° C. | 5 min |
| Step2 | 94° C. | 1 min |
| Step3 | 59.3° C. | 1 min |
| Step4 | 68° C. | 1 min 30 sec |
| Step5 | 68° C. | 10 min |
| Step6 | 10° C. | Hold |

40 cycles of steps 2-4 were carried out and the PCR products were purified using Qiagen's QIAquick PCR Purification Kit (Qiagen Ltd, Boundary Court, Gatwick Road, Crawley, West Sussex, RH10 9AX, UK) according to the manufacturers instructions. The purified PCR products were examined on agarose gels.

cDNA fragments were then cloned in to the pET30 Xa/LIC vector (Novagen), transformed into Nova Blue chemically competent E. coli cells, and plated on to a prewarmed kanamycin (+) selection plate. After an overnight incubation at 37° C., kanamycin-resistant colonies were selected and grown up in kanamycin containing LB medium. Plasmid DNA was isolated using the Plasmid Mini Kit (Qiagen). Confirmation of the presence and correct orientation of the inserts was determined by restriction analysis and sequencing of the construct.

Purified plasmid DNA, which had been confirmed to be of the correct sequence and orientation, was transformed into chemically competent BL21 Star (DE3) One Shot E. coli cells and grown overnight at 37° C. 2 ml of an over-night culture were used to innoculate 100 ml of LB, 30 μg/ml kanamycin, and the cultures incubated at 37° C., 220 rpm until the cell density reached an optical density of 0.6 (approximately 3 hours). Expression of the recombinant protein was then induced with IPTG (1 mM) for 5 hours.

Bacteria were harvested by centrifugation at 4500 rpm for 10 minutes and the pellets lysed in lysis buffer (10 ml Bugbuster (Novagen), 10 μl Benzonase (Novagen), 0.4 μl lysozyme (Novagen) and 100 μl 1M imadazole for 20 minutes at room temperature. Cells were then spun down at 16000 g for 20′ at 4° C. and the supernatant, containing soluble recombinant protein, removed to a clean tube.

Supernatant was added to prewashed Ni-Nta resin at a concentration of 5-10 mg protein per ml of resin and allowed to bind for 1 hour at 4° C. Protein-resin mix was then poured into a column, washed twice in 4 ml of wash buffer (2.5 ml 1M phosphate buffer pH8, 6.25 ml 4M NaCl, 1 ml 1M Imidazole pH8, 0.5 ml 10% Tween 20; made up to 50 mls in n.H₂O) and then eluted in 4×0.5 ml fractions with elution buffer (250 μl 1M Phosphate Buffer pH8, 625 μl 4M NaCl, 1.25 ml 1M Imidazole pH8, 50 μl 10% Tween 20, Made up to 5 mls in n.H₂O). Fractions containing purified protein were detected by SDS-Page and Western blotting using an S-tag HRP conjugate (Novagen). Fractions containing purified recombinant protein were concentrated using YM10 columns (Millipore)

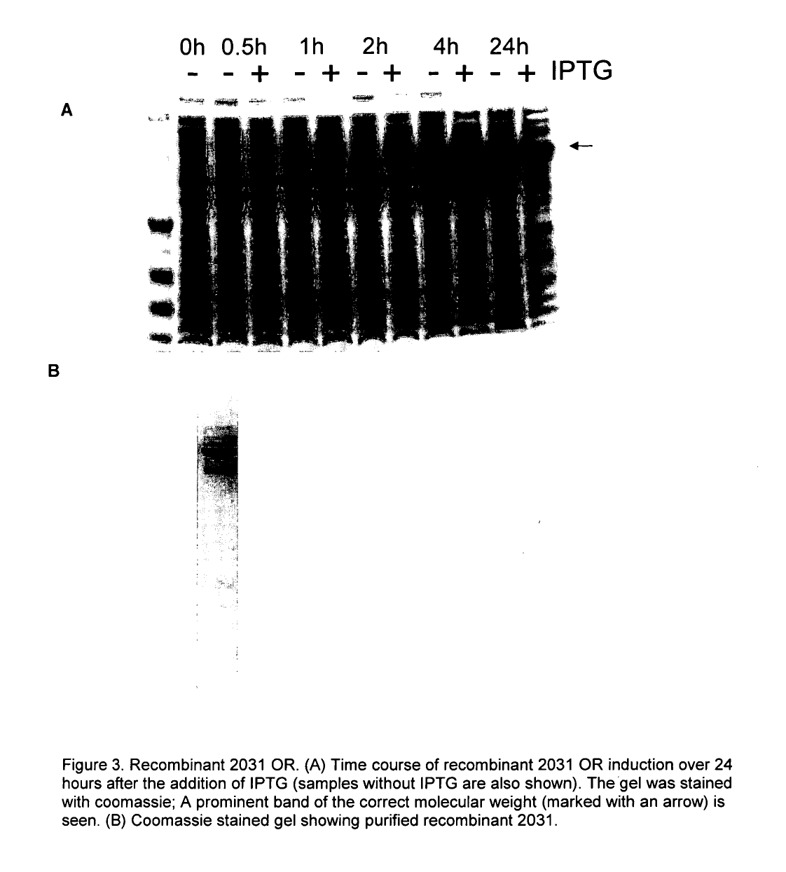

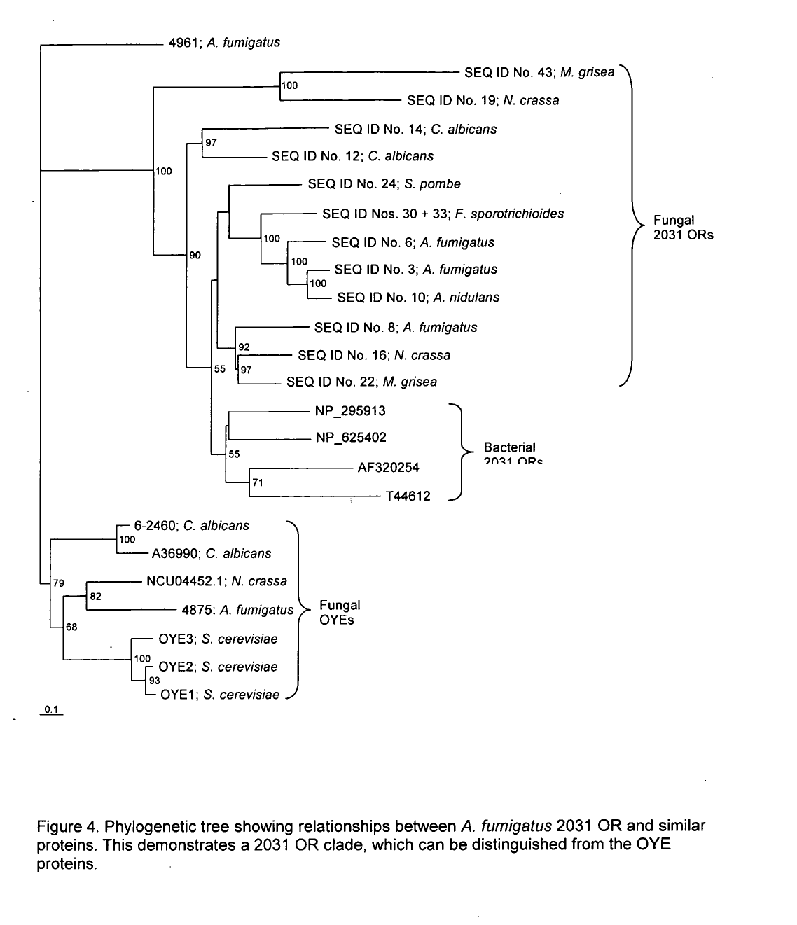

FIG. 3A shows the induction of recombinant 2031 OR expression by IPTG over 24 hours. Protein samples were taken at time points, run on an SDS-PAGE gel and stained with coomassie. By 1 hr a band of the correct size was clearly induced compared to the uninduced samples. The amount of protein increased with longer induction times. FIG. 3B shows a coomassie stained gel of the purified recombinant 2031 OR. Alternative expression systems can be used for expression in bacteria, such as the glutathione S-transferase or mannose-binding fusion-protein system.

Recombinant fragments of other 2031 ORs can be generated using the primer pairs and templates described in Table VIII, or similar primers and other 2031 OR listed in Table III.

TABLE VIII
Primer pairs for the recombinant expression of 2031 OR family proteins

| Species | Template | Primer A | Primer B |
|---|---|---|---|
| A. fumigatus | SEQ ID No. 2 | SEQ ID No. 55 | SEQ ID No. 56 |
| A. fumigatus | SEQ ID No. 5 | SEQ ID No. 57 | SEQ ID No. 58 |
| A. fumigatus | SEQ ID No. 7 | SEQ ID No. 59 | SEQ ID No. 60 |
| A. nidulans | SEQ ID No. 9 | SEQ ID No. 61 | SEQ ID No. 62 |
| C. ablicans | SEQ ID No. 11 | SEQ ID No. 63 | SEQ ID No. 64 |
| M. grisea | SEQ ID No. 21 | SEQ ID No. 65 | SEQ ID No. 66 |

### Example 7

### Oxidoreductase Assay and Inhibitor Screening

### 7.1 Oxidoreductase Assay

The assay for 2031 OR is based on methods described by Abramovitz & Massey (1976, J. Biol. Chem. 251: 5321-5326) and Stott et al. (1993, J. Biol. Chem. 268: 6097-6106) and is based upon the ability of this enzyme to oxidise the pyridine nucleotides NADH and/or NADPH. The peak of absorbance for the reduced form of these cofactors (i.e. NADH and NADPH) is at a wavelength of 340 nm whereas the oxidised forms of the cofactors (i.e. NAD⁺ and NADP⁺) do not absorb at this wavelength. Conversion of NAD(P)H to NAD(P)⁺ can therefore be monitored spectrophotometrically at a wavelength of 340 nm. A similar assay can be employed for all oxidoreductases that use NADH or NADPH as a cofactor.

Assays were carried out in 96-well plates. To each well was added the following; Recombinant 2031 OR (10-1000 ng); 40 μl of 125-2500 μM NADPH; 1 μL 100 mM cyclohexeneone or other substrate, and the volume made up to 200 μL with 0.1 M potassium phosphate pH 7.0. Samples were incubated at room temperature and absorbance measurements were taken at 340 nm every 30 seconds for 10 min. The change in absorbance was expressed as nmoles NADPH oxidised, using the molar extinction coefficient of NADPH and NADH at 340 nm of 6270 (i.e., a 1M solution has an optical density of 6270 at this wavelength).

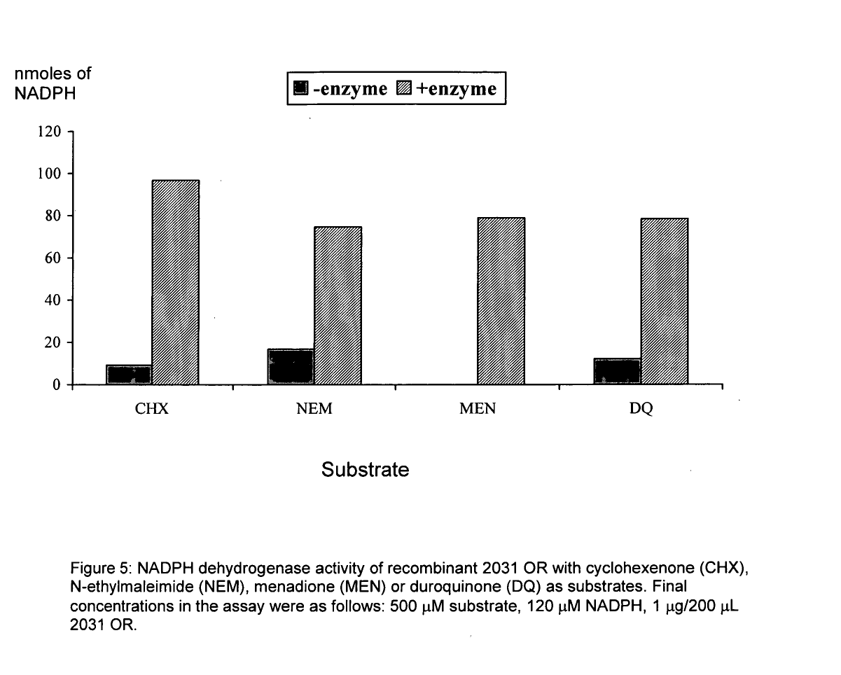

Initial experiments with a variety of potential substrates for recombinant 2031 OR showed that the protein had a functional dehydrogenase activity and determined that cyclohexenone was a better substrate than menadione, duroquinone or N-ethylmaleimide. This is illustrated in FIG. 5. Final concentrations in the assay were as follows: 500 μM substrate, 1 μg/200 μL 2031 OR, 120 μM NADPH.

Although the physiological substrates of 2031 OR remain to be determined, generic oxidoreductase substrates such as ferricyanide, methylene blue, phenazine methosulphate and 2,6-dichlorophenolindophenol may also be used to assay for oxidoreductase activity.

Screens for inhibitors of 2031 OR can be carried out using the assay described above modified by the addition of putative inhibitor substances to the reactions and decreasing the amount of potassium phosphate buffer. Assays can be carried out in 384- or 1536-well plates to increase throughput of the screen.

### 7.2 High-Throughput Screen for the Identification of 2031 OR Inhibitors

2031 OR inhibitors were identified by means of a high-throughput screen. The following reagents were prepared:

Assay plates: Compounds to be tested were dissolved in 100% DMSO (polypropylene vessels), diluted in water and loaded into 384 square well polystyrene plates (10 μl/well). The final DMSO concentration in all assay wells was 5% v/v.

βNADPH (tetrasodium salt)/2-cyclohexen-1-one reagent; Solutions of NADPH (1.2917 mM in 100 mM potassium phosphate buffer, pH7.0) and 2-cyclohexen-1-one (10 mM in 100 mM potassium phosphate buffer, pH7.0) were prepared on the day of the assay and combined in a ratio of 1 part of 2-cyclohexen-1-one solution to 9 parts NADPH solution. Final assay well concentrations for NADPH and 2-cyclohexen-1-one were 465 μM and 400 μM respectively.

2031 OR enzyme: Recombinant enzyme was prepared as described in Example 6 and desalted as follows: 2.5 ml of eluted protein was loaded onto on to a PD10 column (Amersham) equilibrated with 25 ml of 0.1 M KPO₄ pH7. The protein was then eluted with 3.5 ml of 0.1 M KPO₄ pH7. Aliquots of the protein were stored at −80° C. For the iscreen, protein was typically diluted to 5 to 11.25 μg/ml in 100 mM potassium phosphate buffer, pH7.0.

Stop reagent: 0.4 M NaOH in water.

The Km for 2-cyclohexen-1-one, the substrate for 2031 OR in the screening assay, was determined to be 100 μM. To give an increased signal, the screen was carried out using 2-cyclohexen-1-one at 4 times Km. The kinetics of the screen over the prescribed incubation time were such that reaction progress curves were both linear with time and protein concentration. The Z′ value for the screen was equal to 0.77 and thus fully acceptable (Zhang et al., 1999, J. Biomolecular Screening, 4, 67-73). Consistency of signal between wells on plates, plate to plate and screen run to screen run were also acceptable for an HTS regime.

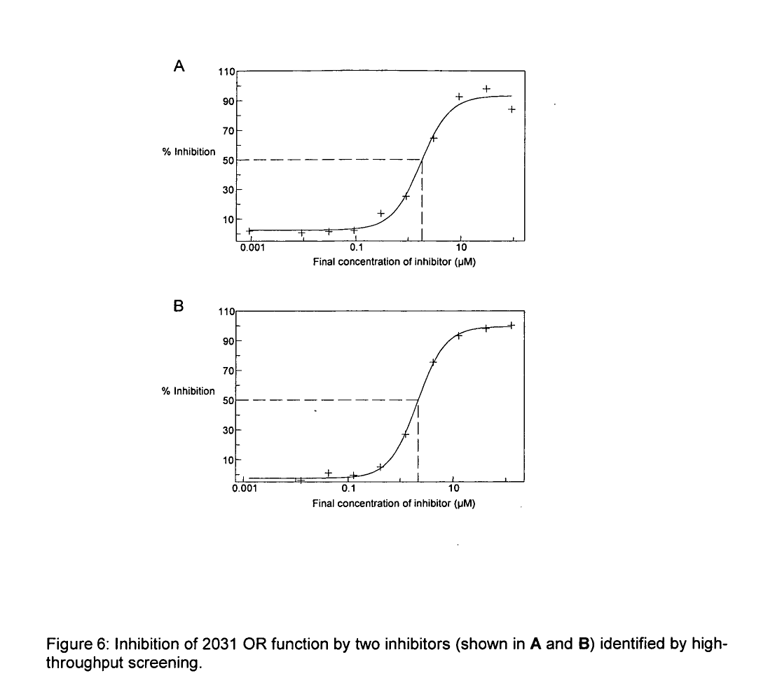

Assays were carried out using Tecan Freedom, Tecan TeMo and PerkinElmer Minitrak robots together with a ThermoLabsystems multidrop 384 and a Tecan Safire automated plate reader. 20 μl of enzyme followed by 20 μl NADPH/2-cyclohexen-1-one solution were added to wells of the microtitre plates containing test compounds. 20 μl of 100 mM potassium phosphate buffer, pH7.0 was used for a duplicate set of plates for background no-enzyme controls; DMSO (diluted in the same way as solubilised compound stocks) was used for no-compound controls. Plates were incubated at room temperature for 30 minutes after which 25 μl of 0.4 M NaOH stop reagent was added. Plates were read at 340 nm on a Tecan Safire plate reader and data processed using ‘in-house’ created Excel spreadsheets to convert raw data into percent inhibition data. Secondary screens were carried out to measure dose response data for selected compounds, using essentially the same protocol as the primary screen. The secondary screen used the Excelfit version 3 software (IDBS), with sigmoidal model 606, to graph appropriate inhibition values and determine IC50 data for compounds tested. FIG. 6 shows typical results for 2 inhibitory compounds (A and B) identified by the primary screen and then assayed in the secondary screen.

Identification of the correct stop reagent for the HTS assay was not trivial. Initially, a chemical inhibitor of the system was sought to terminate the reactions in a pH independent manner, but it was found that NaOH offered more benefits than originally anticipated, in that it not only overcame the buffering in the reaction to fully terminate the reaction, but also afforded a much greater protection for un-reacted NADPH. It is known that high levels of NaOH convert NADP, a product of the reaction which does not absorb at 340 nm, to a fluorescent product, which would interfere with the 340 nm readings taken (Passonneau and Lowry, 1993, Enzymatic analysis, a practical guide, pp. 3-21 and p 381. 1993 The Humana Press Inc. NJ USA.). Therefore, the NaOH level used in the HTS assay was chosen such that the amount of fluorescence from NADP conversion was reduced to an insignificant level, whilst fully terminating the reaction. The greater stability of the NADPH afforded by the use of NaOH meant that instead of immediate plate readings, plates could be read up to at least 20 hours post reaction termination (no further extended time points were investigated). This was an obvious advantage in that larger screens could be run. Plates stored for spectrophotometric reading were sealed with self adhesive film and stored in the dark.

### Example 8

### Method for Detecting Fungal Infection

The sequences described in the invention were exploited to diagnose fungal infections. Samples from patients potentially carrying an infection with A. fumigatus, A. nidulans, or C. albicans or rice leaves or stem potentially infected with M. grisea, or of alfalfa infected with C. trifolii, or wheat infected with F. graminearum, F. sporotrichioides, or M. graminicola, or other organisms, are processed to extract DNA using the DNAeasy Tissue kit or QIAamp DNA Blood Mini kit (Quiagen, Crawley, UK), although other DNA preparation methods are available and suitable.

Once DNA has been prepared, PCR reactions are set up as follows:

### Reaction Mix:

12.5 μl 2× ReddyMix PCR mastermix (ABgene)
1 μl μl primer A (5 pmol)
1 μl primer B (5 pmol)
5 μl template DNA
5.5 μl nuclease-free water
Suitable primer pairs are given in the table IX below:

TABLE IX
Primer pairs for PCRs to diagnose fungal infection.

| Species | Template | Primer A¹ | Primer B¹ |
|---|---|---|---|
| A. fumigatus | SEQ ID No. 1 | SEQ ID No. 67 (94) | SEQ ID No. 68 |
| | | | (286) |
| A. fumigatus | SEQ ID No. 4 | SEQ ID No. 69 (239) | SEQ ID No. 70 |
| | | | (450) |
| A. fumigatus | SEQ ID No. 7 | SEQ ID No. 71 (1097) | SEQ ID No. 72 |
| | | | (1271) |
| C. ablicans | SEQ ID No. 11 | SEQ ID No. 73 (103) | SEQ ID No. 74 |
| | | | (277) |
| M. grisea | SEQ ID No. 20 | SEQ ID No. 75 (385) | SEQ ID No. 76 |
| | | | (620) |

Figures in brackets after SEQ ID No. indicate the base in the template at which the primer starts.

Appropriate controls include; (i) template DNA but no primers; primers but no template (negative controls); (ii) cDNA encoding fungal 2031 OR or DNA from cultured fungi instead of patient DNA (positive control).

PCR reactions are run as follows:

| | | |
|---|---|---|
| Step1 | 95° C. | 5 min |
| Step2 | 95° C. | 1 min |
| Step3 | 53° C. | 1 min 30 sec |
| Step4 | 72° C. | 1 min 30 sec |
| Step5 | 72° C. | 10 min |
| Step6 | 4° C. | Hold |

30 cycles of steps 2-4 are carried out and the PCR products examined on agarose gels. The production of a band of the correct molecular weight is diagnostic of the presence of the particular fungus. It may be additionally necessary to carry out diagnostic restriction digests of the PCR products. If necessary, PCR products are subcloned into a vector, such as pGEM-Teasy (Promega), and sequenced to verify that the PCR products are from the appropriate fungus.

Alternatively, the presence of an infection with A. fumigatus, A. nidulans, C. albicans or M. grisea, C. trifolii, F. graminearum, F. sporotrichioides or M. graminicola, or other organisms is detected by means of antibodies raised against the fungal protein. One suitable means is the use of a capture ELISA. Here, microtitre plates are coated with a monoclonal antibody raised against the fungal protein. Then the plates are incubated with diluted patient samples, or appropriate protein extracts of samples (particularly if the samples are biopsies or plant tissues). Plates are then incubated with a polyclonal antibody (again against the fungal protein). Finally, binding of the second antibody was detected by means of an enzyme-coupled or fluorescently-labelled antibody directed against the polyclonal. In practise, two monoclonal or polyclonal antibodies or various combinations may be used.

### Example 9

### Production of an Antibody

Antibodies against the fungal 2031 ORs will be of considerable use as diagnostic reagents (see example 8 above). As an immunogen, recombinant domains are used (as described in Example 6). Alternatively, synthetic proteins encoding regions either unique to the individual 2031 ORs, or likely to provide cross-reactivity within a set of ORs, a set of species, or a range of genera are used. Peptides may need to be conjugated to carrier proteins before immunization.

Preimmune sera from animals to be immunised are screened against the immunogen to ensure that there is no endogenous cross reactivity. Animals (typically sheep, rabbits or mice) are then immunised. For polyclonal antibody production, the resulting sera is affinity purified using the immunogen cross-linked to a chromatography matrix. Alternatively, purification of the antibody fraction from the serum, e.g. using protein G or protein A cross-linked to a matrix, may be sufficient. Monoclonal antibody production proceeded by methods familiar to those skilled in the art.

The specificities of the resulting polyclonal and/or monoclonal antibodies are checked by ELISA and/or western blotting using the immunogen, related constructs or whole cell lysates and extracts as targets. Negative controls, such as other ORs, different constructs or different species are also employed to test specificity and/or to determine the range of species and/or genus cross-reactivity.

### Example 10

### Production of Fungi with 2031 OR Genes Functionally Disabled

A BAC (bacterial artificial chromosome) clone library containing the A. fumigatus genome, partially digested with BamHI and inserted into the vector pBACe3.6 was purchased from the Sanger Centre, Cambridge, UK. The BAC clone containing the gene to be inactivated is identified by bioinformatics (BLAST searching of Sanger BAC and related databases) and the glycerol stock of the clone grown up in 50 ml LB, 20 μg/ml chloramphenicol at 37° C. overnight. The overnight culture is centrifuged at 4,500 rpm for 15 min. The bacterial pellet is resuspended in 4 ml of Buffer P1 (Qiagen plasmid miniprep kit) and then 4 ml of buffer P2 (Qiagen plasmid miniprep kit, lysis buffer) is added and mixed gently by inverting 3-6 times. Proteins and genomic DNA are precipitated by adding 4 ml of buffer P3 (Qiagen plasmid miniprep kit, neutralizing buffer) and incubating on ice for 10 minutes. Following the centrifugation of the mixture at 4500 rpm for 30 min, the supernatant is transferred into a 50 ml falcon tube, an equal volume of phenol/chlorophorm (1:1) mixture is added, and the mixture centrifuged for 15 min at 4500 rpm. The supernatant is then transferred into an Oakridge tube and 0.7 volumes isopropanol are added. After mixing, the tube is centrifuged at 10,000 rpm (Beckman centrifuge, rotor JA-17) for 30 min at 4° C. The resulting pellet is washed with 2 ml 70% ethanol at the same speed. The resulting BAC DNA is resuspended in 100 μl buffer EB.

The transposition reaction is carried out as follows. 7 μl purified BAC, 1 μl transposon pZVK2 (an engineered plasmid the sequence of which is given as SEQ ID No. 81), containing the mosaic ends of pMOD2 (Epicenter), a kanamycin resistance gene and a Zeocin resistance gene under the control of fungal promoter) and 1 μl EZ:TN transposase (Epicenter) are incubated at 37° C. for two hrs after which 1 μl stop solution (1% SDS) is added and the mixture heated to 70° C. for 10 minutes. Electrocompetent GeneHogs E. coli cells (Invitrogen) are then transformed with the transposed BAC, the cells plated onto LB agar, 25 μg/ml kanamycin, 20 μg/ml chloramphenicol, and plates incubated overnight at 37° C.

At least 96 colonies are picked and grown up in 96-well plates in 2×LB (double concentrated LB), 20 μg/ml chloramphenicol, at 37° C. overnight. BAC DNA is then purified using the Millipore montage 96 BAC KIT using a MWG ROBOSEQ 4200 robot. BACs containing the transposon inserted into the gene of interest are identified by PCRs both spanning the gene of interest and extending from the transposon into the BAC. Insertion into the gene of interest is manifested as an increase in product size. Southern blots are also carried out to ensure that the transposon has only inserted once into the BAC.

The BAC is then linearised using a restriction enzyme determined to cut in the vector backbone but not the BAC DNA, and used to transform A. fumigatus strain Af293. A. fumigatus (haploid) protoplasts are prepared using 5% Glucanex (Novo Nordisk A/S) solution (in 0.6 M KCl) and shaking for 2 h at 80 rpm in 30° C. The protoplasts are washed with 0.6 M KCl and then with STC (Sorbitol, Tris, CaCl₂). The washed protoplasts are diluted in STC to 10⁵/ml and 100 μl transferred into 14 ml falcon tubes. 7 μl of linearised BAC are added to the tube and the whole mixture incubated on ice for 20 min. Transformation is carried out by adding 200 μl of PEG 8000 solution (60% w/v, pH 7.5) drop-wise over 2 min and then adding 800 μl PEG. The mixture is left at room temperature for 20 min. Transformed protoplasts are washed with STC, resuspended in 1 ml STC, spread onto CM-sorbitol-Zeocin (250 μg/ml) plates and incubated at 37° C.

After 4-10 days of incubation, zeocin resistant colonies are picked and checked for presence of the knocked-out gene by PCR using primers which specifically amplify the whole gene of interest. Usually 10-20 transformants are checked. The ectopic integration of the BAC gives two bands by PCR, one for the endogenous gene and one for the BAC/transposon construct, which has a higher molecular weight. Replacement of the endogenous gene with the transposon-modified gene results in a single band of higher molecular weigh by PCR. If none of the transformants show the disrupted endogenous gene, the gene of interest may be essential, with the knock-out cells having died and only cells where replacement is unsuccessful surviving. In this case, the transformation is carried out on diploids using the same method of transformation. Essentiality of the gene is then tested by rehaploidisation, and examining the segregation pattern in haploids.

### Example 11

### Rescue of MycoBank Transformant with the 2031 Oxidoreductase Gene

### 11.1 Preparation of the 2031 OR Construct

The 2031 OR gene with NheI overhangs was prepared by PCR using the primer pair;

### SEQ ID No 98 and SEQ ID No. 99.

### PCR Reaction:

2.5 μl 10×PCR buffer

0.5 μl dNTPs

2 μl MgSO₄

1 μl forward primer (SEQ ID No. 98)

1 μl reverse primer (SEQ ID No. 99)

1 μl gDNA

Made up to 25 μl with n.H₂O

PCR Cycle: (1) 94° C., 5′; (2) 94° C., 1′; (3) 50° C., 1′; (4) 68° C. 1′30 s; (5) 68° C., 10′; (6) 8° C., Pause; Cycles 2 to 4 were repeated 40 times

The finished amplicon (˜1260 bp) was run out on a 1% agarose gel, the appropriate band was cut out and purified using the Qiagen gel extraction kit and eluted off the column in 30 μl H₂O. The amplicon was ligated into pGEM Teasy using the following reaction mixture:

5 μl 2× ligation buffer

1 μl pGEM Teasy vector

either 1, 2 or 3 μl of insert

1 μl μl T4 DNA ligase

Reaction made up to 10 μl with n.H₂O

The ligation reaction was incubated overnight in the fridge

2 μl of each ligation reaction was transformed by heatshock at 42° C. into promega 96 select cells. After transformation, cells were incubated in SOC for 1 h at 37° C., 220 rpm. 50 and 150 μl aliquots were then spread over LB-Amp (100 μg/ml), IPTG-Xgal plates and left at 37° C. overnight. Positive clones were identified by blue/white screening and were isolated and screened by PCR for correct insertion of the 2031 OR insert using the above primers. Positive clones were sent away to MWG for sequence analysis.

11.2 Cloning of 2031 OR into the CbhB-Zeo Vector

Plasmid DNA for 2031 OR in pGem Teasy (as described in 11.1) was digested overnight at 37° C. with NheI. The 2031 OR insert fragment was then gel purified using the Qiagen gel extraction kit and ligated into CbhB-Zeo vector. This vector was constructed from pUC19 with the A. fumigatus CbhB promoter and terminator and the zeocin resistance gene.

### Ligation:

1 μl of T4 DNA ligase

1 μl of 10× ligase buffer

1 μl of CbhB vector (linearised and alkaline phoshatase treated)

1 μl of insert

6 μl n.H₂O

Ligation reaction was left in the fridge overnight.

2 μls of each ligation reaction was transformed by electroporation at 2.5 Kvolts, 200Ω, 25 μF into Genehog cells. After transformation, cells were incubated in SOC for 1 h at 37° C., 220 rpm. 50 and 150 μl aliquots were then spread over LB-Amp (100 μg/ml) plates and left at 37° C. overnight. Positive clones were isolated and screened by PCR for the correct insertion of the insert by PCR as above. Positives were sent to MWG for sequence analysis.

11.3 Transformation into Mycobank Mutant 2031

The CbhB-Zeo-2031 plasmid was digested with ScaI overnight at 37° C. Linearised plasmid was then run out on a 1% agarose gel and purified using the Qiagen gel extraction kit. Plasmid DNA was eluted in 30 μs of nH₂O.

Mycobank mutant 2031 AF293 spores were swollen for 6 h at 37° C., 300 rpm, centrifuged 3500 rpm, 5′ and resuspended in ice-cold nH₂O, Spores were spun again, 3500 rpm, 5′ then resuspended in 12.5 ml of YED medium and incubated for 1 h at 30° C., 100 rpm. Spores were then counted and resuspend in EB buffer to a final concentration of 5×10⁷ spores per ml. 50 μl of swollen spores were then transformed with 1-10 μl of linearised CbhB-Zeo-2031 plasmid DNA at 1 Kvolt, 400Ω, 25 μF. Spores were transferred in to YED buffer and left for 90′ at 37° C., 100 rpm. 100 and 200 μl aliquots were then spread out on to CM-Zeocin (200 μg/ml) plates and incubated at 37° C. for 2-3 days.

Positive transformants on the CM-Zeo plates were picked into 5 ml of SAB broth and incubated overnight at 37° C., 220 rpm. Biomass was then filtered and collected on to Whatman paper. DNA was extracted using the Fast prep kit and cleaned up over a Qiagen miniprep DNA column. DNA was eluted off column in 30 μl of nH₂O.

PCR Screening was performed using the following primer sets:

Set A: Ox7race_for (SEQ ID No. 51)+CbhBtR (SEQ ID No. 100)

Set B: Ox6race_rev (SEQ ID No. 50)+CbhBpF (SEQ ID No. 101)

### PCR Reaction:

12.5 μl 2× Reddy mix

1 μl each primer, from se A or B

1 μl plasmid DNA

Made up to 25 uL with water

PCR Cycle: (1) 94° C., 5′; (2) 94° C., 1′; (3) 56° C., 1′; (4) 72° C. 1′30 s; (5) 72° C., 10′; (6) 8° C., Pause; Cycles (2) to (4) were repeated 40 times

Positive transformants which were demonstrated to have CbhB-Zeo-2031 in Mycobank mutant 2031 were put through the rehaploidation process to test their ability to grow on hygromycin compared with the untransformed mycobank mutant 2031. We found that the lethal 2031 phenotype was rescued by the insertion of the CbhB-Zeo-2031 plasmid, confirming the essentially of 2031 OR.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. Method of identifying an anti-fungal agent which targets an essential protein or gene of a fungus comprising contacting a candidate substance with
(i) a NADH:flavin oxidoreductase protein which comprises the sequence shown by SEQ ID NO:3,
(ii) a NADH:flavin oxidoreductase protein which is a homologue of (i) and which comprises the sequence shown by SEQ ID NO: 8, 12, 14, 19, 24, 42, 44, 83 or 85,
(iii) a protein which has 50% identity with (i) or (ii),
(iv) a protein comprising a fragment of (i), (ii) or (iii) which fragment has a length of at least 50 amino acids,
(v) a polynucleotide that comprises sequence which encodes (i), (ii), (iii) or (iv),
(vi) a polynucleotide comprising sequence which has at least 70% identity with the coding sequence of (v),
and determining whether the candidate substance binds or modulates (i), (ii), (iii), (iv), (v) or (vi), wherein binding or modulation of (i), (ii), (iii), (iv), (v) or (vi) indicates that the candidate substance is an anti-fungal agent.

2. Method according to claim 1 wherein (iii) or (iv) have an oxidoreductase activity.

3. Method according to claim 1 wherein (i), (ii), (iii) or (iv) comprise one or more of the motifs defined by regions 1 to 11 in FIGS. 1 and 2.

4. Method according to claim 1 comprising carrying out a redox reaction in the presence and absence of the candidate substance to determine whether the candidate substance inhibits the oxidoreductase activity of a protein as defined in any one of the preceding claims, wherein the redox reaction is carried out by contacting said protein with NADH or NADPH; and an electron acceptor, under conditions in which in the absence of the candidate substance the protein catalyses reduction of the electron acceptor.

5. Method according to claim 1 wherein (iii) is a protein comprising the sequence of any of the following: SEQ ID NO: 6, 10, 16, 22, 27, 30, 33, 35, 38, 40.

6. Method according to claim 1 wherein the (i) or (ii) is an oxidoreductase of Aspergillus flavus, Aspergillus fumigatus; Aspergillus nidulans, Aspergillus niger; Aspergillus parasiticus; Aspergillus terreus; Blumeria graminis; Candida albicans, Candida cruzei; Candida glabrata; Candida parapsilosis; Candida tropicalis; Colletotrichium trifolii; Cryptococcus neoformans, Encephalitozoon cuniculi; Fusarium graminarium; Fusarium solani; Fusarium sporotrichoides; Leptosphaeria nodorum, Magnaporthe grisea, Mycosphaerella graminicola; Neurospora crassa; Phytophthora capsici; Phytophthora infestans; Plasmopara viticola; Pneumocystis jiroveci; Puccinia coronata; Puccinia graminis; Pyricularia oryzae; Pythium ultimum; Rhizoctonia solani; Schizzosaccharomyces pombe; Trichophyton interdigitale; Trichophyton rubrum; or Ustilago maydis.

7. Method according to claim 1 which further comprises formulating the identified anti-fungal agent into a agricultural or pharmaceutical composition.

8. Method according to claim 1 which further comprises killing or impairing the growth of a fungus by contacting the fungus with the identified anti-fungal agent.

9. (canceled)

10. (canceled)

11. Method of detecting the presence of a fungus in a sample comprising detecting the presence in the said sample of a protein or polynucleotide as defined in claim 1.

13-25. (canceled)

26. A composition of matter comprising:
(a) An isolated protein or polynucleotide as defined in claim 1, or
(b) A vector comprising a polynucleotide as defined in claim 1, or
(c) A recombinant cell comprising a polynucleotide as defined in claim 1, or
(d) An organism which is transgenic for a polynucleotide as defined in claim 1, or
(e) An organism which has been genetically engineered to render a polynucleotide or protein as defined in claim 1 non-functional or inhibited, or
(f) An antibody which is specific for a protein as defined in claim 1, or
(g) A fungus which has been killed, or whose growth has been impaired, by inhibition of the expression or activity of a protein or polynucleotide as defined in claim 1.

27. A method
(a) for preventing or treating a fungal infection comprising administering an anti-fungal agent identified by the method of claim 1 or a protein or polynucleotide as defined in claim 1; or
(b) of killing, or impairing the growth of, a fungus comprising inhibiting the expression or activity of a polynucleotide or protein as defined in claim 1.

28. A method according to claim 27 wherein the fungus has infected a human, animal or plant individual.

29. A method of obtaining
(a) a protein as defined in claim 1 comprising expressing the protein from a polynucleotide as defined in claim 1, or
(b) a polynucleotide as defined in claim 1 comprising synthesis of the polynucleotide by condensation of nucleotides.
